(19) United States
(12) Patent Application Publication
(10) Pub. No.: US 2011/0081348 A1
(43) Pub. Date: Apr. 7, 2011

(54) FUNGAL SIGNALLING AND METABOLIC ENZYMES

(76) Inventors: Nina Brogden, Greater Manchester (GB); Michael John Bromley, Manchester (GB); Paul David Carr, Greater Manchester (GB); Sarah Jane Kaye, Greater Manchester (GB); Jason David Oliver, Manchester (GB); Daniel Scott Tuckwell, Manchester (GB)

(21) Appl. No.: 11/720,664

(22) PCT Filed: Dec. 1, 2005
(86) PCT No.: PCT/GB2005/004604

§ 371 Date: Aug. 27, 2007

(30) Foreign Application Priority Data
Dec. 1, 2004 (GB) 0426390.1
Oct. 17, 2005 (GB) 0521062.0

(51) Int. Cl. A61K 39/395; C07K 16/14; A61K 31/7088; A61K 38/48; A01N 63/02; A01N 37/18; A01P 3/00; A61P 31/10; G01N 33/53; C12N 15/63; C07H 21/00; C12N 9/88; C12N 1/14; C12N 1/15; C12N 1/21

(52) U.S. Cl. 424/139.1; 530/387.9; 514/44 A; 424/94.63; 435/7.8; 435/320.1; 536/23.2; 435/232; 435/254.1; 435/254.11; 435/252.33

(57) ABSTRACT

Method of identifying an anti-fungal agent which targets as an essential protein or gene of a fungus comprising contacting a candidate substance with (i) a protein which comprises the sequence shown by SEQ ID NOS: 3, 6, 9, 12, 15, 18, 21, 24, 27, 30, 33, 36, 39, 42, 45, 48, 50, 53, 56, 59, 61 or 63, or (ii) a protein which has 60% identity with (i), or (iii) a protein comprising a fragment of (i) or (ii) which fragment has a length of at least 50 amino acids, or (iv) a polynucleotide that comprises a sequence which encodes (i), (ii) or (iii), or (v) a polynucleotide comprising a sequence which has at least 70% identity with the coding sequence of (iv), and determining whether the candidate substance binds or modulates (i), (ii), (iii), (iv), or (v), wherein binding or modulation of (i), (ii), (iii), (iv), or (v) indicates that the candidate substance is an anti-fungal agent.

### FIELD OF THE INVENTION

The present invention relates to a method of screening for an anti-fungal agent and to fungal genes involved in signalling and metabolism.

### BACKGROUND OF THE INVENTION

Invasive fungal infections are well recognised as diseases of the immunocompromised host. Over the last twenty years there have been significant rises in the number of recorded instances of fungal infection (Groll et al., 1996, J Infect 33, 23-32). In part this is due to increased awareness and improved diagnosis of fungal infection. However, the primary cause of this increased incidence is the vast rise in the number of susceptible individuals. This is due to a number of factors including new and aggressive immunosuppressive therapies, increased survival in intensive care, increased numbers of transplant procedures and the greater use of antibiotics worldwide.

In certain patient groups, fungal infection occurs at high frequency; lung transplant recipients have a frequency of up to 20% colonisation and infection with a fungal organism and fungal infection in allogenic hoemopoetic stem transplant recipients is as high as 15% (Ribaud et al., 1999, Clin Infect Dis. 28:322-30).

Currently only four classes of antifungal drug are available to treat systemic fungal infections. These are the polyenes (e.g., amphotericin B), the azoles (e.g., ketoconazole or itraconazole) the echinocandins (e.g., caspofungin) and flucytosine.

The polyenes are the oldest class of antifungal agent being first introduced in the 1950's. The exact mode of action remains unclear but polyenes are only effective against organisms that contain sterols in their outer membranes. It has been proposed that amphotericin B interacts with membrane sterols to produce pores allowing leakage of cytoplasmic components and subsequent cell death.

Azoles function by the inhibition of 14α-demethylase via a cytochrome P450-dependent mechanism. This leads to a depletion of the membrane sterol ergosterol and the accumulation of sterol precursors resulting in a plasma membrane with altered fluidity and structure.

Echinocandins work by inhibiting the cell wall synthesis enzyme β-glucan synthase, leading to abnormal cell wall formation, osmotic sensitivity and cell lysis.

Flucytosine is a pyrimidine analogue interfering with cellular pyrimidine metabolism as well DNA, RNA and protein synthesis. However widespread resistance to flucyotosine limits its therapeutic use.

It can be seen that, to date, the currently available antifungal agents act primarily against only two cellular targets; membrane sterols (ployenes and azoles) and β-glucan synthase (echinocandins).

Resistance to both azoles and polyenes has been widely reported leaving only the recently introduced echinocandins to combat invasive fungal infections. As the use of echinocandins increases, resistance by fungi will inevitably occur.

The identification of new classes of anti-fungal agent with novel modes of action is required to ensure positive therapeutic outcomes for patients in the future. Novel fungal-specific genes are likely to present the best opportunity for the development of effective novel anti-fungal agents. In particular it is highly desirable that target genes are present in a range of fungi, but absent from humans, and fungal-specific genes involved in metabolism and signalling would be valuable candidates. The inventors have exploited the availability of fungal and mammalian genomes to identify such genes which are thus suitable as targets for the development of anti-fungal drugs.

### SUMMARY OF THE INVENTION

The inventors have found a set of twelve genes which are present in fungi but not humans. This finding allows the identification of anti-fungal agents based on their ability to target these genes.

The invention provides a set of twelve proteins which can be used to screen for anti-fungal agents. In particular a set of twelve proteins from Aspergillus fumigatus (see Table I) is provided.

The inventors have found two Aspergillus fumigatus genes which resemble the single S. cerevisiae ILV3 gene. ILV3 is essential in S. cerevisiae for the biosynthesis of the branched amino acids leucine, isoleucine and valine, but this enzyme is absent from animals, making it a good target for an antifungal. This gene has not been used before as a target for the discovery of an antifungal agent, nor have recombinant ILV3 proteins been synthesised. Surprisingly the inventors have found that two A. fumigatus ILV3-like genes have to be knocked out to render the organism inviable.

The invention therefore provides ILV3-like genes of fungi (see Tables I and II) which can be used either individually or together (as pairs) to screen for antifungal agents.

Accordingly the invention provides the following:
a method of identifying an anti-fungal agent which targets a protein or gene of a fungus comprising contacting a candidate substance with
   (i) a protein which comprises the sequence shown by SEQ ID NOs: 3, 6, 9, 12, 15, 18, 21, 24, 27, 30, 33 or 36
   (ii) a protein which has 60% identity with (i),
   (iii) a protein comprising a fragment of (i) or (ii) which fragment has a length of at least 50 amino acids,
   (iv) a polynucleotide that comprises sequence which encodes (i), (ii) or (iii),
   (v) a polynucleotide comprising sequence which has at least 70% identity with the coding sequence of (iv),
      and determining whether the candidate substance binds or modulates (i), (ii), (iii), (iv) or (v), wherein binding or modulation of (i), (ii), (iii), (iv) or (v) indicates that the candidate substance is an anti-fungal agent,
use of (i), (ii), (iii), (iv) or (v) as defined above to identify or obtain an anti-fungal agent,
a method of identifying an anti-fungal agent which targets ILV3 genes of fungi comprising contacting a candidate substance with
   (i) a protein which comprises the sequence shown by SEQ ID NOs: 12, 21, 39, 42, 45, 48, 50, 53, 56, 59, 61 or 63
   (ii) a protein which has at least 60% identity with (i),
   (iii) a protein comprising a fragment of (i) or (ii) which fragment has a length of at least 50 amino acids,
   and determining whether the candidate substance binds or modulates (i), (ii) or (iii), wherein binding or modulation of (i), (ii) or (iii) indicates that the candidate substance is an anti-fungal agent,
use of (i), (ii), or (iii) as defined above to identify or obtain an anti-fungal agent,
a method of identifying an anti-fungal agent which targets ILV3 genes of fungi comprising contacting a candidate substance with
   (i) a protein which comprises the sequence shown by SEQ ID NOs: 21, 42, 45, 53 or 56
   (ii) a protein which has at least 60% identity with (i), (iii) a protein comprising a fragment of (i) or (ii) which fragment has a length of at least 50 amino acids, contacting the same substance with
   (iv) a protein which comprises the sequence shown by SEQ ID NOs: 12, 39, 48, 50 or 59
   (v) a protein which has at least 60% identity with (iv),
   (vi) a protein comprising a fragment of (i) or (ii) which fragment has a length of at least 50 amino acids,
      and determining whether the candidate substance binds or modulates (i), (ii) or (iii), and (iv), (v) or (vi), wherein binding or modulation of (i), (ii) or (iii), and (iv), (v) or (vi) indicates that the candidate substance is an anti-fungal agent,
the above method wherein the first screen in carried out with SEQ ID NOs: 12, 39, 48, 50 or 59 and the second screen with SEQ ID NOs: 21, 42, 45, 53 or 56.
use of (i), (ii), or (iii), and (iv), (v) or (vi) as defined above to identify or obtain an anti-fungal agent,—use of an anti-fungal agent identified by the method of the invention in the manufacture of a medicament for prevention or treatment of fungal infection,
an isolated protein or polynucleotide of the invention,
an organism which is transgenic for a polynucleotide of the invention,
an organism which has been genetically engineered to render a polynucleotide or protein of the invention non-functional or inhibited,
an antibody which is specific for a protein of the invention,
a method for preventing or treating a fungal infection comprising administering an anti-fungal agent identified by the screening method of the invention, and
a fungus which has been killed, or whose growth has been impaired, by inhibition of the expression or activity of a protein or polynucleotide of the invention.

TABLE I
A. fumigatus sequences claimed and their relationship to sequences given in the sequence listing

| | | | | SEQ ID Nos. | | |
|---|---|---|---|---|---|---|
| A. nidulans | | A. fumigatus match | | | cDNA/ | |
| Protein | Predicted function | Contig; E-value | Bases of match | gDNA¹ | mRNA² | Protein |
| AN0392.2 | Exonuclease/endonuclease/ | 1375.1; 1e⁻¹⁰⁵ | 343874 . . . 345398 | 1: (1 . . . 204, 249 . . . 505, 556 . . . | 2 | 3 |
| | phosphatase | | | 837, 894 . . . 1067, 1123 . . . 1525) | | |
| AN0829.2 | 3′5′, cyclic nucleotide | 1; 1e⁻¹⁰⁶ | 1743929 . . . 1745527 | 4: (1 . . . 619, 767 . . . 1599) | 5 | 6 |
| | phosphodiesterase | | | | | |
| AN3636.2 | Phosphatidyl inositol-specific | 29; 0.0 | 763057 . . . 764490 | 7 | 8 | 9 |
| | phospholipase C | | | | | |
| AN4058.2 | ILV3 | 1352.1; 0.0 | 207620 . . . 209584 | 10; (1 . . . 159, 229 . . . 442, | 11 | 12 |
| | | | | 512 . . . 1965) | | |
| AN4426.2 | Tyrosine phosphatase | 26: 3e⁻⁴⁹ | 336336 . . . 337166 | 13: (1 . . . 89, 143 . . . 286, | 14 | 15 |
| | | | | 340 . . . 535, 589 . . . 830) | | |
| AN4941.2 | Transporter | 1352.1; 2e⁻⁹⁵ | 506553 . . . 507563 | 16: (1 . . . 68, 124 . . . 381, | 17 | 18 |
| | | | | 441 . . . 1011) | | |
| AN6346.2 | ILV3 | 34; 0.0 | 1848981 . . . 1848993 | 19: (1 . . . 56, 130 . . . 1845, | 20 | 21 |
| | | | | 1915 . . . 1960) | | |
| AN6680.2 | GPCR | 6; 1e⁻¹⁴⁹ | 523423 . . . 525126 | 22: (1 . . . 79, 152 . . . 235, 301 . . . | 23 | 24 |
| | | | | 464, 511 . . . 649, 707 . . . 1704) | | |
| AN7298.2 | Exoribonuclease | 34; 1e⁻¹⁰³ | 2624338 . . . 2625294 | 25: (1 . . . 423, 478 . . . 957) | 26 | 27 |
| AN8262.2 | GPCR (related to cAMP receptor | 1364.0; 1e⁻¹¹⁹ | 654170 . . . 655630 | 28: (1 . . . 257, 334 . . . 535, | 29 | 30 |
| | subtype 2 of D. discoideum) | | | 608 . . . 1111, 1189 . . . 1461) | | |
| AN8990.2 | Amino acid transporter/ | 1366.2; 0.0 | 141352 . . . 143154 | 31: (1 . . . 558, 682 . . . 881, | 32 | 33 |
| | permease | | | 936 . . . 1516, 1586 . . . 1803) | | |
| AN9156.2 | Exonuclease/endonuclease/ | 1360.2; 0.0 | 1053939 . . . 1055909 | 34: (1 . . . 676, 730 . . . 1098, | 35 | 36 |
| | phosphatase | | | 1149 . . . 1594, 1639 . . . 1971) | | |

¹Numbers after SEQ ID Nos. correspond to bases of genomic DNA encoding the protein in cases where introns are present.
²RNA sequences are given in the sequence listing with Thymidine (T), although it is understood that in vivo Uridine (U) would be present.

TABLE II
Fungal ILV3 genes

| | | | | Deposited | |
|---|---|---|---|---|---|
| Species | gDNA | cDNA | protein | sequence | Group¹ |
| A. fumigatus | 10 | 11 | 12 | | II |
| “ILV1352” | | | | | |
| A. fumigatus | 19 | 20 | 21 | | I |
| “ILV34” | | | | | |
| A. nidulans | 37 | 38 | 39 | AN4058² | II |
| A. nidulans | 40 | 41 | 42 | AN6346 | I |
| F. graminearum | 43 | 44 | 45 | FG02056.1 | I |
| F. graminearum | 46 | 47 | 48 | FG02717.1 | II |
| M. grisea | 49 | 49 | 50 | MG01139.4³ | II |
| M. grisea | 51 | 52 | 53 | MG05345.4 | I |
| N. crassa | 54 | 55 | 56 | NCU04579 | I |
| N. crassa | 57 | 58 | 59 | NCU05683.1 | II |
| C. albicans | 60 | 60 | 61 | XP_721948 | |
| S. cerevisiae | 62 | 62 | 63 | NP_012550 | |

¹Groups I sequences cluster with A. fumigatus sequence SEQ ID No. 21; group II sequences cluster with A. fumigatus SEQ ID No. 12.
²AN4058 sequence differs from that deposited in the publicly available database and was repredicted from genomic DNA based on alignment with other ILV genes.
³Only the C-terminal sequence of this gene could be predicted.

### DETAILED DESCRIPTION OF THE INVENTION

As mentioned above the invention relates to use of particular proteins and polynucleotide sequences (termed “proteins of the invention” and “polynucleotides of the invention” herein), including homologues and/or fragments of the fungal proteins and polynucleotides, to identify anti-fungal agents.

A protein or polynucleotide of the invention may be defined by similarity in sequence to a another member of the family. This similarity may be based on percentage identity (for example to the sequences shown in the sequence listing).

A protein or polynucleotide of the invention may be an ILV3 or ILVD protein, defined as a dihydroxy acid dehydratase, or as a protein which shows homology to SEQ ID No. 21, or as a protein which matches the ILVD_EDD Pfam profile.

The protein or polynucleotide of the invention may align with other proteins or polynucleotides of the invention (as shown in SEQ ID Nos. 1-63).

The protein or polynucleotide of the invention may be in isolated form (such as non-cellular form), or, in the case of membrane-associated proteins, as a membrane preparation, for example when used in the method of the invention. The polynucleotide may comprise native, synthetic or recombinant polynucleotide, and the protein may comprise native, synthetic or recombinant protein. The polynucleotide or protein may comprise combinations of native, synthetic or recombinant polynucleotide or protein, respectively. The polynucleotides and proteins of the invention may have a sequence which is the same as, or different from, naturally occurring polynucleotides and proteins.

It is to be understood that the term “isolated from” may be read as “of” herein. Therefore references to polynucleotides and proteins being “isolated from” a particular organism include polynucleotides and proteins which were prepared by means other than obtaining them from the organism, such as synthetically or recombinantly.

Preferably, the polynucleotide or protein, is isolated from a fungus, more preferably a filamentous fungus, even more preferably an Ascomycete.

Preferably, the polynucleotide or protein, is isolated from an organism selected from Aspergillus; Blumeria; Candida; Colletotrichium; Cryptococcus; Encephalitozoon; Fusarium; Histoplasma, Leptosphaeria; Magnaporthe; Mycosphaerella; Neurospora; Phytophthora; Plasmopara; Pneumocystis; Pyricularia; Pythium; Puccinia; Rhizoctonia; Saccharomyces, Schizosaccharomyces, Trichophyton; and Ustilago.

Preferably, the polynucleotide or protein, is isolated from Aspergillus. Preferably, the polynucleotide or protein, is isolated from an organism selected from the species Aspergillus flavus; Aspergillus fumigatus; Aspergillus nidulans; Aspergillus niger; Aspergillus parasiticus; Aspergillus terreus; Blumeria graminis; Candida albicans; Candida cruzei; Candida glabrata; Candida parapsilosis; Candida tropicalis; Colletotrichium trifolii; Cryptococcus neoformans; Encephalitozoon cuniculi; Fusarium graminarium; Fusarium solani; Fusarium sporotrichoides; Histoplasma capsulata; Leptosphaeria nodorum; Magnaporthe grisea; Mycosphaerella graminicola; Neurospora crassa; Phytophthora capsici; Phytophthora infestans; Plasmopara viticola; Pneumocystis jiroveci; Puccinia coronata; Puccinia graminis; Pyricularia oryzae; Pythium ultimum; Rhizoctonia solani; Saccharomyces cerevisiae; Schizosaccharomyces pombe; Trichophyton interdigitale; Trichophyton rubrum; and Ustilago maydis.

Preferably, the polynucleotide or protein, is isolated from Aspergillus fumigatus, preferably the protein, may be isolated from A. fumigatus AF293.

Variants of the above mentioned polynucleotides and proteins are also provided, and are discussed below.

In one embodiment, the protein of the invention may comprise an amino acid sequence substantially as set out and independently selected from any of SEQ ID Nos: 3, 6, 9, 12, 15, 18, 21, 24, 27, 30, 33, 36, 39, 42, 45, 48, 50, 53, 56, 59, 61, 63 or variants thereof.

The polynucleotide of the invention may comprise DNA, such as genomic DNA. The polynucleotide may comprise a sequence substantially as set out and independently selected from any of SEQ ID Nos. 1, 4, 7, 10, 13, 16, 19, 22, 25, 28, 31, 34, 37, 40, 43, 46, 49, 51, 54, 57, 60, 62 or complements, or variants thereof.

The polynucleotide may comprise RNA, preferably mRNA, preferably spliced mRNA. Preferably, the polynucleotide comprises substantially the sequence shown as SEQ ID Nos 2, 5, 8, 11, 14, 17, 20, 23, 26, 29, 32, 35, 38, 41, 44, 47, 49, 52, 55, 58, 60, 62, or a complement, or a variant thereof.

Preferably, the protein is encoded by the regions of sequences SEQ ID Nos. 1, 4, 7, 10, 13, 16, 19, 22, 25, 28, 31, 34, 37, 40, 43, 46, 49, 51, 54, 57, 60 or 62 as described in the column “gDNA” in Tables I or II, or a complement, or a variant thereof.

Preferably, the isolated polynucleotide comprises substantially a nucleotide sequence independently selected from the regions and sequences given in the column “gDNA” in Tables I or II.

Preferably, the protein is encoded by a polynucleotide which polynucleotide comprises substantially a sequence independently selected from at least one of the regions and sequences given in the column “gDNA” in Tables I or II, or a complement or, a variant thereof.

Preferably, the polynucleotide encodes a protein which comprises substantially the amino acid sequences SEQ ID Nos: 1, 4, 7, 10, 13, 16, 19, 22, 25, 28, 31, 34, 39, 42, 45, 48, 50, 53, 56, 59, 61, 63 or a variant thereof.

By the term “native amino acid/polynucleotide/protein”, is meant an amino acid, polynucleotide or protein produced naturally from biological sources either in vivo or in vitro.

By the term “synthetic amino acid/polynucleotide/protein”, is meant an amino acid, polynucleotide or protein which has been produced artificially or de novo using a DNA or protein synthesis machine known in the art.

By the term “recombinant amino acid/polynucleotide/protein”, is meant an amino acid, polynucleotide or protein which has been produced using recombinant DNA or protein technology or methodologies which are known to the skilled technician.

The term “variant”, and the terms “substantially the amino acid/polynucleotide/protein sequence” are used herein to refer to related sequences. As discussed below such related sequences are typically homologous to (share percentage identity with) a given sequence, for example over the entire length of the sequence or over a portion of a given length. The related sequence may also be a fragment of the sequence or of a homologous sequence. A variant protein may be encoded by a variant polynucleotide.

By the term “variant”, and the terms “substantially the amino acid/polynucleotide/protein sequence”, we mean that the sequence has at least 30%, preferably 40%, more preferably 50%, and even more preferably, 60% sequence identity with the amino acid/polynucleotide/protein sequences of any one of the sequences referred to. A sequence which is “substantially the amino acid/polynucleotide/peptide sequence” may be the same as the relevant sequence.

Calculation of percentage identities between different amino acid/polynucleotide/protein sequences may be carried out as follows. A multiple alignment is first generated by the ClustaiX program (pairwise parameters: gap opeining 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calcluated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polynucleotide/protein seqences may be synthesised de novo, or may be native amino acid/polynucleotide/protein sequence, or a derivative thereof.

An amino acid/polynucleotide/protein sequence with a greater identity than 65% to any of the sequences referred to is also envisaged. An amino acid/polynucleotide/protein sequence with a greater identity than 70% to any of the sequences referred to is also envisaged. An amino acid/polynucleotide/protein sequence with a greater identity than 75% to any of the sequences referred to is also envisaged. An amino acid/polynucleotide/protein sequence with a greater identity than 80% to any of the sequences referred to is also envisaged. Preferably, the amino acid/polynucleotide/protein sequence has 85% identity with any of the sequences referred to, more preferably 90% identity, even more preferably 92% identity, even more preferably 95% identity, even more preferably 97% identity, even more preferably 98% identity and, most preferably, 99% identity with any of the referred to sequences.

The above mentioned percentage identities may be measured over the entire length of the original sequence or over a region of 15, 20, 50 or 100 amino acids/bases of the original sequence. In a preferred embodiment percentage identity is measured with reference to SEQ ID Nos. 3, 6, 9, 12, 15, 18, 21, 24, 27, 30, 33, 36, 39, 42, 45, 48, 50, 53, 56, 59 61 or 63. Preferably the variant protein has at least 40% identity, such as at least 60% or at least 80% identity with SEQ ID Nos. 3, 6, 9, 12, 15, 18, 21, 24, 27, 30, 33, 36, 39, 42, 45, 48, 50, 53, 56, 59, 61 or 63 or a portion of one of these.

Alternatively, a substantially similar nucleotide sequence will be encoded by a sequence which hybridizes to the sequences shown in SEQ ID Nos. 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 51, 52, 54, 55, 57, 58, 60, 62, or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar protein may differ by at least 1, but less than 5, 10, 20, 50 or 100 amino acids from the sequences shown in SEQ ID Nos. 3, 6, 9, 12, 15, 18, 21, 24, 27, 30, 33,. 36, 39, 42, 45, 48, 50, 53, 56, 59, 61 or 63. Such differences may each be additions, deletions or substitutions.

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change.

Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. Certain organisms, including Candida are known to use non-standard codons compared to those used in the majority of eukaryotes. Any comparisons of polynucleotides and proteins from such organisms with the sequences given here should take these differences into account.

In accurate alignment of protein or DNA sequences the trade-off between optimal matching of sequences and the introduction of gaps to obtain such a match is important. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantitate the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Use of the Align program is also preferred (Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYLIP.

Other modifications in protein sequences are also envisaged and within the scope of the claimed invention, i.e. those which occur during or after translation, e.g. by acetylation, amidation, carboxylation, GPI-linkage, myristoylation, phosphorylation, proteolytic cleavage or linkage to a ligand.

The term “variant”, and the terms “substantially the amino acid/polynucleotide/protein sequence” also include a fragment of the relevant polynucleotide or protein sequences, including a fragment of the homologous sequences (which have percentage identity to a specified sequence) referred to above. A polynucleotide fragment will typically comprise at least 10 bases, such as at least 20, 30, 50, 100, 200, 500 or 1000 bases. A protein fragment will typically comprise at least 10 amino acids, such as at least 20, 30, 50, 80, 100, 150, 200, 300, 400 or 500 amino acids. The fragments may lack at least 3 amino acids, such as at least 10, 20 or 30 amino acids of the amino acids from either end of the protein.

The invention provides methods of screening which may be used to identify modulators of the proteins or polynucleotides of the invention, such as inhibitors of expression or activity of the proteins or polynucleotides of the invention. In one embodiment of the method a candidate substance is contacted with a protein or polynucleotide of the invention and whether or not the candidate substance binds or modulates the protein or polynucleotide is determined.

The modulator may promote (agonise) or inhibit (antagonise) the activity of the protein. A therapeutic modulator (against fungal infection) will inhibit the expression or activity of protein or polynucleotide of the invention.

The method may be carried out in vitro (inside or outside a cell) or in vivo. The method may be carried out on a cell, or cell culture extract, or cell extract or cell-membrane fraction. The cell may or may not be a cell in which the polynucleotide or protein is naturally present. The cell may or may not be a fungal cell, or may or may not be a cell of any of the fungi mentioned herein. The protein or polynucleotide may be present in a non-cellular form in the method, thus the protein may be in the form of a recombinant protein purified from a cell.

Any suitable binding or activity assay may be used. Methods which determine whether a candidate substance is able to bind the protein or polynucleotide may comprise providing the protein or polynucleotide to a candidate substance and determining whether binding occurs, for example by measuring the amount of the candidate substance which binds the protein or polynucleotide. The binding may be determined by measuring a characteristic of the protein or polynucleotide that changes upon binding, such as spectroscopic changes. The binding may be determined by measuring reaction substrate or product levels in the presence and absence of the candidate and comparing the levels.

The assay format may be a ‘band shift’ system. This involves determining whether a test candidate advances or retards the protein or polynucleotide on gel electrophoresis relative to the absence of the compound.

The method may be a competitive binding method. This determines whether the candidate is able to inhibit the binding of the protein or polynucleotide to an agent which is known to bind to the protein or polynucleotide, such as an antibody specific for the protein, or a substrate of the protein.

Whether or not a candidate substance modulates the activity of the protein may be determined by providing the candidate substance to the protein under conditions that permit activity of the protein, and determining whether the candidate substance is able to modulate the activity of the product.

The activity which is measured may be any of the activities of the proteins of the invention mentioned herein, including; endonuclease, exonuclease, exoribonuclease, G-protein coupled receptor, ILV3/dihydroxyacid dehydratase, kinase, phosphatase, phosphatididylinositol-specific phospholipase C, phosphodiesetrase, protein tyrosine phosphatase, ion transport or small molecule transport/permease activities. In one embodiment the screening method comprising carrying out a reaction in the presence and absence of the candidate substance to determine whether the candidate substance inhibits the activity of the protein of the invention.

ILV3 activity can be measured as follows: An ILV3 protein is incubated with a substrate molecule such as dihydroxy valeric acid, dihydroxy methylvaleric acid, another dihydroxy acid, or a polyhydroxy acid (such as threonic acid or 2,3,4,5-tetrahydroxy pentanoic acid), and the appearance of a keto acid product measured either directly or indirectly. Direct measurement can be carried out by means of spectrophotometry, for example at 240 nm, whereas indirect measurement can be carried out by reacting the keto acid with semicarbazide and measuring the appearance of product by spectrophotometry, for example at 250 nm, or by reacting the keto acid with 2,4-dinitrophenylhydrazine and measuring the reaction products by spectrophotometry at 540-550 nm. This assay may be used as a screen for inhibitors of filamentous fungal ILV3s by (a) adding to the assay putative inhibitor compounds and looking for a decrease in product, and (b) carrying out the assay firstly with a group I ILV3 (Table II) and then carrying out the assay with a group II ILV3 (or vice versa) and identifying compounds that inhibit in both assays. The assay can be carried out with recombinant A. fumigatus ILV34 and ILV1352 (Table II).

ILV3 inhibitors may also be identified by the above assay using a single ILV3 protein such as from any of the following species: organism selected from the species Aspergillus flavus; Aspergillus fumigatus; Aspergillus nidulans; Aspergillus niger; Aspergillus parasiticus; Aspergillus terreus; Blumeria graminis; Candida albicans; Candida cruzei; Candida glabrata; Candida parapsilosis; Candida tropicalis; Colletotrichium trifolii; Cryptococcus neoformans; Encephalitozoon cuniculi; Fusarium graminarium; Fusarium solani; Fusarium sporotrichoides; Histoplasma capsulata; Leptosphaeria nodorum; Magnaporthe grisea; Mycosphaerella graminicola; Neurospora crassa; Phytophthora capsici; Phytophthora infestans; Plasmopara viticola; Pneumocystis jiroveci; Puccinia coronata; Puccinia graminis; Pyricularia oryzae; Pythium ultimum; Rhizoctonia solani; Saccharomyces cerevisiae; Schizosaccharomyces pombe; Trichophyton interdigitale; Trichophyton rubrum; and Ustilago maydis.

In a further embodiment of the method, a candidate substance is contacted with a cell heterozygous for an underexpressed, mutated, disrupted or deleted copy or copies of the gene or genes, and the extent to which the candidate substance inhibits growth of the cell is determined by any suitable means and compared to the effects of the candidate substance on cells homozygous for unaltered copies of the gene. The heterozygous cell will show greater sensitivity to substances that inhibit the gene or its gene product.

Suitable candidate substances which can tested in the above methods include antibody products (for example, monoclonal and polyclonal antibodies, single chain antibodies, chimeric antibodies and CDR-grafted antibodies). Furthermore, combinatorial libraries, defined chemical identities, peptide and peptide mimetics, oligonucleotides and natural product libraries, such as display libraries (e.g. phage display libraries) may also be tested. The candidate substances may be chemical compounds. Batches of the candidate substances may be used in an initial screen of, for example, ten substances per reaction, and the substances from batches which show inhibition tested individually.

According to a further aspect of the present invention, there is provided a polynucleotide or protein of the invention for use as a medicament or in diagnosis.

The polynucleotide or protein may be modified prior to use, preferably to produce a derivative or variant thereof. The polynucleotide or protein may be derivatised. The protein may be modified by epitope tagging, addition of fusion partners or purification tags such as glutathione S-transferase, multiple histidines or maltose binding protein, addition of green fluorescent protein, covalent attachment of molecules including biotin or fluorescent tags, incorporation of selenomethionine, inclusion or attachment of radioisotopes or fluorescent/non-fluorescent lanthanide chelates. The polynucleotide may be modified by methylation or attachment of digoxygenin (DIG) or by addition of sequence encoding the above tags, proteins or epitopes.

Preferably, the medicament is adapted to retard or prevent a fungal infection. The fungal infection may be in human, animal or plant. The polynucleotide or protein may be used for the development of a drug. The polynucleotide or protein may be used in, or for the generation of, a molecular model of said polynucleotide or said protein.

According to a further aspect of the present invention, there is provided use of a polynucleotide or protein of the invention for the preparation of a medicament for the treatment of a fungal infection.

The polynucleotide or protein may be modified prior to use, preferably to produce a derivative or variant thereof. The polynucleotide or protein may be derivatised. The polynucleotide or protein may not be modified or derivatised.

Preferably, the medicament is adapted to retard or prevent a fungal infection. The treatment may comprise retarding or preventing fungal infection. Preferably, the drug and/or medicament comprises an inhibitor. Preferably, the drug or medicament is adapted to inhibit expression and/or activity of the polynucleotide or a fragment thereof, and/or the function of the protein or a fragment thereof.

Preferably, the fungal infection comprises an infection by a fungus, more preferably an Ascomycete, and even more preferably, an organism selected from the genera Aspergillus; Blumeria; Candida; Colletotrichium; Cryptococcus; Encephalitozoon; Fusarium; Histoplasma, Leptosphaeria; Magnaporthe; Mycosphaerella; Neurospora; Phytophthora; Plasmopara; Pneumocystis; Pyricularia; Pythium; Puccinia; Rhizoctonia, Trichophyton; and Ustilago.

Preferably, the fungal infection comprises an infection by an organism selected from the genera Aspergillus.

Preferably, the fungal infection comprises an infection by an organism selected from the species Aspergillus flavus; Aspergillus fumigatus; Aspergillus nidulans; Aspergillus niger; Aspergillus parasiticus; Aspergillus terreus; Blumeria graminis; Candida albicans; Candida cruzei; Candida glabrata; Candida parapsilosis; Candida tropicalis; Colletotrichium trifolii; Cryptococcus neoformans; Encephalitozoon cuniculi; Fusarium graminarium; Fusarium solani; Fusarium sporotrichoides; Histoplasma capsulata; Leptosphaeria nodorum; Magnaporthe grisea; Mycosphaerella graminicola; Neurospora crassa; Phytophthora capsici; Phytophthora infestans; Plasmopara viticola; Pneumocystis jiroveci; Puccinia coronata; Puccinia graminis; Pyricularia oryzae; Pythium ultimum; Rhizoctonia solani; Trichophyton interdigitale; Trichophyton rubrum; and Ustilago maydis.

Preferably, the fungal infection comprises an infection by Aspergillus fumigatus.

According to a further aspect of the present invention, there is provided a recombinant DNA molecule or vector comprising a polynucleotide of the invention.

The recombinant DNA molecule or vector may comprise an expression cassette. Preferably, the recombinant DNA molecule or vector comprises an expression vector. Preferably, the polynucleotide sequence is operatively linked to an expression control sequence. A suitable control sequence may comprise a promoter, an enhancer etc.

According to another aspect of the present invention, there is provided a cell containing a polynucleotide, recombinant DNA molecule or vector of the invention.

The cell may be transformed or transfected with the polynucleotide, recombinant DNA molecule or vector by suitable means. Preferably, the cell produces a recombinant protein of the invention.

The invention also provides an organism which is transgenic for the polynucleotide of the invention (whose cells may be the same as the cells of the invention mentioned herein). Such an organism is typically a fungus, such as any genera or species of fungus mentioned herein. The organism may be a microorganism, such as a bacterium, virus or yeast. The organism may be a plant, or animal (including birds and mammals), such as any of the animals mentioned herein.

The organism may be produced by introduction of the polynucleotide of the invention into a cell of the organism, and in the case of a multicellular organism allowing the cell to grow into a whole organism.

According to a further aspect of the present invention, there is provided a cell in which a polynucleotide or protein of the invention is non-functional and/or inhibited. The cell may be of, or present in, a multicellular organism.

The cell may be a mutant cell. The cell is typically a fungal cell, such as of any genera or species of fungus mentioned herein. A preferred means of generating the cell is to modify the polynucleotide of the invention, such that the polynucleotide is non-functional. This modification may be to cause a mutation, which disrupts the expression or function of a gene product. Such mutations may be to the nucleic acid sequences that act as 5′ or 3′ regulatory sequences for the polynucleotide, or may be a mutation introduced into the coding sequence of the polynucleotide. Functional deletion of the polynucleotide may be, for example, by mutation of the polynucleotide in the form of nucleotide substitution, addition or, preferably, nucleotide deletion.

The polynucleotide may be made non-functional and/or inhibited by:

(i) shifting the reading frame of the coding sequence of the polynucleotide;
(ii) adding, substituting or deleting amino acids in the protein encoded by the polynucleotide; or
(iii) partially or entirely deleting the DNA coding for the polynucleotide and/or the upstream and downstream regulatory sequences associated with the polynucleotide.
(iv) inserting DNA into the coding or non-coding regions.

A preferred means of introducing a mutation into a polynucleotide is to utilize molecular biology techniques specifically to target the polynucleotide which is to be mutated. Mutations may be induced using a DNA molecule. A most preferred means of introducing a mutation is to use a DNA molecule that has been especially prepared such that homologous recombination occurs between the target polynucleotide and the DNA molecule. When this is the case, the DNA molecule, which may be double stranded, may contain base sequences similar or identical to the target polynucleotide to allow the DNA molecule to hybridize to (and subsequently recombine with) the target.

In the case of ILV3 proteins the mutant cell may contain mutations of two different ILV3 genes, where the function of either or both gene products may be inhibited or abolished.

It is also possible to provide a cell in which the polynucleotide is non-functional and/or inhibited without introducing a mutation into the gene or its regulatory regions. This may be done by using specific inhibitors. Examples of such inhibitors include agents that prevent transcription of the polynucleotide, or prevent translation, expression or disrupt post-translational modification. Alternatively, the inhibitor may be an agent that increases degradation of the gene product (e.g. a specific proteolytic enzyme). Equally, the inhibitor may be an agent which prevents the polynucleotide product from functioning, such as neutralizing antibodies. The inhibitor may also be an antisense oligonucleotide, or any synthetic chemical capable of inhibiting expression of the gene or the stability and/or function of the protein. The inhibitor may also be a protein which interacts with a protein of the invention prevent its function. The inhibitor may also be an RNA molecule which causes inhibition by RNA interference. In one embodiment the antisense polynucleotide or RNA molecule which causes RNA interference is an example of a polynucleotide of the invention.

According to a further aspect, there is provided an antibody exhibiting immunospecificity for a protein of the invention. The antibody may be used as a diagnostic reagent.

The antibody may be monoclonal or polyclonal, and may be raised in mouse, rat, rabbit, chicken, turkey, horse, goat or donkey. The antibody may be raised against one of the proteins of the invention, or may be raised against proteolytic or recombinant fragments.

For the purposes of this invention, the term “antibody”, unless specified to the contrary, includes fragments which bind a protein of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

### Administration

The formulation of any of the therapeutic substances (e.g. proteins, polynucleotides or modulators) mentioned herein will depend upon factors such as the nature of the substance and the condition to be treated. Any such substance may be administered in a variety of dosage forms. It may be administered orally (e.g. as tablets, troches, lozenges, aqueous or oily suspensions, dispersible powders or granules), parenterally, subcutaneously, intravenously, intramuscularly, intrasternally, transdermally or by infusion techniques. The substance may also be administered as suppositories. A physician will be able to determine the required route of administration for each particular patient.

Typically the substance is formulated for use with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

A therapeutically effective non-toxic amount of substance is administered. The dose may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg, preferably from about 0.1 mg/kg to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

### Agricultural Use

Modulators identified by the method of the invention may be administered to plants in order to prevent or treat fungal infections. The modulators are normally applied in the form of compositions together with one or more agriculturally acceptable carriers or diluents and can be applied to the crop area or plant to be treated, simultaneously or in succession with further compounds.

The modulators of the invention can be applied together with carriers, surfactants or application-promoting adjuvants customarily employed in the art of formulation. Suitable carriers and diluents correspond to substances ordinarily employed in formulation technology, e.g. natural or regenerated mineral substances, solvents, dispersants, wetting agents, tackifiers, binders or fertilizers.

A preferred method of applying the modulators of the present invention or an agrochemical composition which contains them is leaf application. The number of applications and the rate of application depend on the intensity of infection by the fungus. However, the active ingredients can also penetrate the plant through the roots via the soil (systemic action) by impregnating the locus of the plant with a liquid composition, or by applying the compounds in solid form to the soil, e.g. in granular form (soil application). The active ingredients may also be applied to seeds (coating) by impregnating the seeds either with a liquid formulation containing active ingredients, or coating them with a solid formulation. In special cases, further types of application are also possible, for example, selective treatment of the plant stems or buds.

The active ingredients are used in unmodified form or, preferably, together with the adjuvants conventionally employed in the art of formulation, and are therefore formulated in known manner to emulsifiable concentrates, coatable pastes, directly sprayable or dilutable solutions, dilute emulsions, wettable powders, soluble powders, dusts, granulates, and also encapsulations, for example, in polymer substances. Like the nature of the compositions, the methods of application, such as spraying, atomizing, dusting, scattering or pouring, are chosen in accordance with the intended objectives and the prevailing circumstances. Advantageous rates of application are normally from 50 g to 5 kg of active ingredient (a.i.) per hectare (“ha”, approximately 2.471 acres), preferably from 100 g to 2 kg a.i./ha, most preferably from 200 g to 500 g a.i./ha.

The formulations, compositions or preparations containing the active ingredients and, where appropriate, a solid or liquid adjuvant, are prepared in known manner, for example by homogeneously mixing and/or grinding active ingredients with extenders, for example solvents, solid carriers and, where appropriate, surface-active compounds (surfactants).

Suitable solvents include aromatic hydrocarbons, preferably the fractions having 8 to 12 carbon atoms, for example, xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, monomethyl or monoethyl ether, ketones such as cyclohexanone, strongly polar solvents such as N-methyl-2-pyrrolidone, dimethyl sulfoxide or dimethyl formamide, as well as epoxidized vegetable oils such as epoxidized coconut oil or soybean oil; or water.

The solid carriers used e.g. for dusts and dispersible powders, are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable nonsorbent carriers are materials such as calcite or sand. In addition, a great number of pregranulated materials of inorganic or organic nature can be used, e.g. especially dolomite or pulverized plant residues.

Depending on the nature of the active ingredient to be used in the formulation, suitable surface-active compounds are nonionic, cationic and/or anionic surfactants having good emulsifying, dispersing and wetting properties. The term “surfactants” will also be understood as comprising mixtures of surfactants.

Suitable anionic surfactants can be both water-soluble soaps and water-soluble synthetic surface-active compounds. Suitable soaps are the alkali metal salts, alkaline earth metal salts or unsubstituted or substituted ammonium salts of higher fatty acids (chains of 10 to 22 carbon atoms), for example the sodium or potassium salts of oleic or stearic acid, or of natural fatty acid mixtures which can be obtained for example from coconut oil or tallow oil. The fatty acid methyltaurin salts may also be used.

More frequently, however, so-called synthetic surfactants are used, especially fatty sulfonates, fatty sulfates, sulfonated benzimidazole derivatives or alkylarylsulfonates. The fatty sulfonates or sulfates are usually in the form of alkali metal salts, alkaline earth metal salts or unsubstituted or substituted ammoniums salts and have a 8 to 22 carbon alkyl radical which also includes the alkyl moiety of alkyl radicals, for example, the sodium or calcium salt of lignonsulfonic acid, of dodecylsulfate or of a mixture of fatty alcohol sulfates obtained from natural fatty acids. These compounds also comprise the salts of sulfuric acid esters and sulfonic acids of fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnaphthalenesulfonic acid, or of a naphthalenesulfonic acid/formaldehyde condensation product. Also suitable are corresponding phosphates, e.g. salts of the phosphoric acid ester of an adduct of p-nonylphenol with 4 to 14 moles of ethylene oxide.

Non-ionic surfactants are preferably polyglycol ether derivatives of aliphatic or cycloaliphatic alcohols, or saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols.

Further suitable non-ionic surfactants are the water-soluble adducts of polyethylene oxide with polypropylene glycol, ethylenediamine propylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit.

Representative examples of non-ionic surfactants are nonylphenolpolyethoxyethanols, castor oil polyglycol ethers, polypropylene/polyethylene oxide adducts, tributylphenoxypolyethoxyethanol, polyethylene glycol and octylphenoxyethoxyethanol. Fatty acid esters of polyoxyethylene sorbitan and polyoxyethylene sorbitan trioleate are also suitable non-ionic surfactants.

Cationic surfactants are preferably quaternary ammonium salts which have, as N-substituent, at least one C₈-C₂₂ alkyl radical and, as further substituents, lower unsubstituted or halogenated alkyl, benzyl or lower hydroxyalkyl radicals. The salts are preferably in the form of halides, methylsulfates or ethylsulfates, e.g. stearyltrimethylammonium chloride or benzyldi(2-chloroethyl)ethylammonium bromide.

The surfactants customarily employed in the art of formulation are described, for example, in “McCutcheon's Detergents and Emulsifiers Annual”, MC Publishing Corp. Ringwood, N.J., 1979, and Sisely and Wood, “Encyclopaedia of Surface Active Agents,” Chemical Publishing Co., Inc. New York, 1980.

The agrochemical compositions usually contain from about 0.1 to about 99% preferably about 0.1 to about 95%, and most preferably from about 3 to about 90% of the active ingredient, from about 1 to about 99.9%, preferably from about 1 to 99%, and most preferably from about 5 to about 95% of a solid or liquid adjuvant, and from about 0 to about 25%, preferably about 0.1 to about 25%, and most preferably from about 0.1 to about 20% of a surfactant. Whereas commercial products are preferably formulated as concentrates, the end user will normally employ dilute formulations.

All of the features described herein may be combined with any of the above aspects, in any combination.

Embodiments of the invention will now be described by way of example.

### EXAMPLES

### Example 1

### Identification Fungal-Specific Genes in Aspergillus fumigatus

Ideally, fungal target genes should be present in as broad a range of fungi as possible, but absent from humans. A bioinformatics strategy was devised to identify such potential targets exploiting the availability of fungal and human genomes. Programs were written in PERL, and used publicly available downloaded databases and the BLAST algorithm (Altschul et al., 1990, J. Mol. Biol. 215:403-410).

Predicted proteins from the A. nidulans genome (http://www.broad.mitedu/ftp/pub/annotation/aspergillus/assemblyl/release3.1/asper gillus_nidulans_{—}1_r3.1_proteins.fasta.gz) were blasted against the human refseq proteins (ftp://ftp.ncbi.nih.gov/refseq/H_sapiens/H_sapiens/protein/), and only those proteins without a matching human sequence were kept (i.e. E-value >1e-4). This set was then blasted against N. crassa predicted proteins (http://www.broad.mit.edu/ftp/pub/annotation/neurospora/assembly3/neurospora_{—}3_protein.gz) and only those proteins with a good match (i.e., E-value <1e-10) were kept. The resulting set of 2993 proteins therefore contained genes conserved between filamentous fungi but absent from humans. This set was then blasted against C. albicans orfs (http://www-sequence.stanford.edu/group/candida/download.html) and thereby separated into a set of 819 proteins with good homologs (E-value <1e-10), which can be though of as “pan-fungal” proteins, and the other 2184 proteins, which can be thought of as “filamentous-only” proteins.

The pan-fungal set was examined for enzymes or enzyme families. Surprisingly, four ILV3-like genes were identified, AN4058, AN6346, (Tables I and II), AN5138 and AN7358, each of which had an A. fumigatus ortholog. This contrasts with the presence of a single ILV3 gene in S. cerevisiae. Alignment of the four ILV3 genes with ILV3 genes from other organisms, followed by phylogentic analysis identified the two ILV3 genes given in table I as the closest to the S. cerevisiae ILV3 gene. This was supported by percentage identity values given in Table III., A phosphoinositol phospholipase C was also identified (see Table I).

TABLE III
Percentage identities between ILV3 homologs of Aspergilli

| | ILV1352 | AN4058 | ILV34 | AN6346 | AN5138 | Afl346¹ | AN7358 | Af34_B¹ |
|---|---|---|---|---|---|---|---|---|
| ILV3_Sc² | 55.7 | 53.6 | 64.6 | 64.5 | 34.0 | 33.1 | 25.9 | 26.1 |
| ILV1352 | — | 87.5 | 53.2 | 53.6 | 31.2 | 29.9 | 22.3 | 22.9 |
| AN4058 | | — | 51.9 | 52.0 | 30.5 | 29.7 | 22.1 | 22.6 |
| ILV34 | | | — | 89.0 | 34.7 | 33.0 | 26.5 | 27.2 |
| AN6346 | | | | — | 34.0 | 32.5 | 25.9 | 27.1 |
| AN5138 | | | | | — | 84.0 | 30.2 | 29.4 |
| Afl346 | | | | | | — | 28.9 | 28.4 |
| AN7358 | | | | | | | — | 79.7 |

¹Afl346, A. fumigatus ortholog of AN5138, from contig 1346; Af34_B, A. fumigatus ortholog of AN7358, from contig 34.
²ILV3_Sc; ILV3 from S. cerevisiae

The “pan-fungal” and “filamentous-only” sequence sets were also analysed to identify signalling and metabolic molecules, by searching the data sets with PFAM HMMs (Bateman et al., 2004, Nucl. Acids Res. 32, D138-D141; http://www.sanger.ac.uk/Software/Pfam/), using a PERL script and downloaded HMMs. The HMMs used and the proteins identified in this way are given in Table IV.

TABLE IV
Identification of target molecules by HMM

| | HMM name | | | |
|---|---|---|---|---|
| Protein ID | phosphatases | GPCRs | phosphodiesterases | E-value |
| AN0392.2 | Exo_endo_phos.hmm | | | 8.7e−19 |
| AN0829.2 | | | PDEase_II.hmm | 1.3e−25 |
| AN4426.2 | DSPc.hmm | | | 1.7e−05 |
| AN4941.2 | | 7tm_5.hmm | | 4.9e−05 |
| AN6680.2 | | 7tm_2.hmm | | 9.7e−05 |
| AN7298.2 | Exo_endo_phos.hmm | | | 1.5e−06 |
| AN8262.2 | | 7tm_1.hmm | | 3.5e−07. |
| | | 7tm_2.hmm | | 1.7e−08. |
| | | Dict_CAR.hmm | | 7.8e−07 |
| AN8990.2 | | 7tm_5.hmm | | 6e−06 |
| AN9156.2 | Exo_endo_phos.hmm | | | 1.6e−18 |

The A. fumigatus genes corresponding to the A. nidulans genes were identified as follows: The A. nidulans protein was blasted against the A. fumigatus genome (ftp://ftp.sanger.ac.uk/pub/pathogens/A_jumigatus/AF.contigs.031704) to identify the matching region. The matching gene was predicted from this sequence using Genscan (genes.mit.edu/GENSCAN.html; Settings; organism=vertebrate; Suboptimal exon cutoff=1.00) and/or WISE2 (http://www.ebi.ac.uk/Wise2/). The predicted genes were compared with similar sequences using blast, the multiple alignment programs ClustalX (Thompson et al., 1997, Nucleic Acids Research, 24:4876-4882) and QAlign (Sameth et al., 2003, Bioinformatics 19, 1592-1593; http://www.ridom.de/qaligm), and the alignment editor/viewer Align (Hepperle, D., 2001: Multicolor Sequence Alignment Editor. Institute of Freshwater Ecology and Inland Fisheries, 16775 Stechlin, Germany). Gene structures were visualised and modified using Artemis (http://www.sanger.ac.uk/Software/Artemisi; Rutherford et al., 2000, Bioinformatics 16, 944-945). It was necessary to carefully examine predictions and to compare predicted genes with homologous proteins to arrive at an informed prediction. The resulting genes are given in Tables I and II.

### Example 2

### Production of Gene Knockouts in A. fumigatus

For a gene of interest to be suitable as a anti-fungal drug target, it is necessary to show that it is an essential gene by generating a knock-out strain in which the gene is disabled. First a section of genomic DNA is synthesised by PCR, corresponding to the gene of interest and the 2-3 kb on either side, and the PCR products cloned into pGEMT-easy (Promega). The genomic DNA is then used as the substrate for a tansposition reaction using the Epicentre Tn5 bacterial transposon into which fungal and bacterial selection markers have been inserted. Suitable fungal selection genes are PyrG, hygromycin or zeomycin; suitable bacterial markers are kanamycin or zeomycin. The transposed constructs are then screened by PCR to identify those where the transposon has inserted into the gene. PCR primers are designed either to cover the whole gene, such that insertion of the transposon results in the appearance of a product of higher molecular weight, or to extend from the start or end of the gene into the transposon, such that a product is only obtained when the transposon has inserted.

Once a transposed copy has been identified, the genomic DNA/transposon construct is excised from pGEMT-easy with a restriction enzyme which cuts only in the vector (e.g., NotI or DraI) and then used to transform haploid fungal protoplasts by means of PEG-mediated transformation. The fungi are grown under selective conditions, determined by the marker used, and transformants are picked. These are then screened by PCR using primers specific for the gene of interest: Replacement of the endogenous gene with the transposon-modified gene results in a single band of higher molecular weigh by PCR. Therefore, if the modified gene is observed, the gene is not essential. However, if none of the transformants show gene replacement, the gene of interest may be an essential gene. In this case, the transformation is then carried out on diploids using the same method and essentiality of the gene is tested by rehaploidisation followed by examination of the segregation pattern in haploids.

2.1 Gene Disruption of Aspergillus fumigatus ILV3 Genes

Two Aspergillus fumigatus ILV3-like genes ILV34A and ILV1352 (Tables 1 and II) were knocked out as follows. Initially a ˜6 kbp fragment of genomic sequence was generated for each gene follows.

### 2.1.1. ILV34A Mutant Construct

A PCR was set up with Extensor master mix, A. fumigatus genomic DNA, and primers ILV34A_F1 and ILV34A_R1 (SEQ ID Nos. 72 and 73). The resulting 5889 bp PCR product was gel purified (Qiaquick gel purification kit, Qiagen) and ligated into pGEMTeasy overnight at 4° C. (Promega). 1 μl of the ligation mix was transformed into Electrocompetent E. coli Genehogs (Invitrogen) by electroporation. Transformed cells were plated on LB-Ampicillin-IPTG-Xgal agar plates and incubated at 37° C. overnight. White colonies were picked into LB-ampicillin broth and incubated at 37° C. overnight with shaking at 220 rpm. Plasmid DNA was isolated by Qiaprep miniprep DNA isolation (Qiagen). NotI digestion of the plasmid DNA indicated whether a 5.9 kb insert was present and the presence of ILV34 DNA was confirmed by PCR reactions using the following PCR primer sets: a) SEQ ID Nos 72 and 73; b) SEQ ID No. 74 and 75. Plasmids yielding the following size PCR products were deemed to be pGEMTEasy_ILV34A: a) 5889 bp, b) 1930 bp. A plasmid, pMB4zeo, was constructed that contained the mosaic ends recognised by the TN5 transposase, an Aspergillus fumigatus pyr G sequence and a bacterial zeocin resistance gene. The pyrG cassette was prepared with EcoRI sites flanking the genomic pyrG sequence. This cassette was introduced into the EcoRI site of pMOD2 (Epicentre). A zeocin resistance cassette was sub-cloned from an XbaI-NheI fragment of pEMzeo (Invitrogen) into the XbaI site. pMB4zeo was digested with PshAI and XmnI and the 2551 bp fragment obtained was gel purified. This fragment (PshAI-MB4zeo) contained mosaic ends for transposition, an Aspergillus pyrG cassette and a bacterial zeocin resistance marker. pGEMTEasy_ILV34A was mutated by transposition with the EZ::TN transposase kit (Epicentre) using PshAI-MB4zeo. The following were assembled in a microcentrifuge: 1 μl EZ::TN 10× Reaction Buffer, 1 μl pGEMTEasy_ILV34A, 1 μl PshAI-MB4zeo, 6 μl sterile water, 1 μl EZ::TN Transposase. The reaction mixture was incubated for 2 hours at 37° C. 1 μl EZ::TN 10× Stop Solution was added, mixed and heated for 10 minutes at 70° C. 1 μl of the stopped reaction was transformed into Electrocompetent E. coli Genehogs (Invitrogen) by electroporation. Transformed cells were plated on LB-Ampicillin-zeocin agar plates and incubated at 37° C. overnight. Colonies were picked into LB-ampicillin broth and incubated at 37° C. overnight with shaking at 220 rpm. Plasmid DNA was isolated by Qiaprep miniprep DNA isolation (Qiagen). Plasmids were screened by PCR using primer SEQ ID No. 74 and 80. A plasmid was selected that gave a PCR product of approximately 600 bp indicating that the transposon PshAI-MB4zeo had inserted approximately 600 bp from the ATG start site of the coding sequence, thus disrupting the gene. This plasmid was designated ILV34A_KO33. The plasmid was digested with NotI and the 8.4 kb fragment gel purified. This fragment was used for fungal transformation.

### 2.1.2 ILV1352 Mutant Construct

A BAC containing a genomic copy of ILV 1352 was isolated and used as a template for a PCR with Extensor master mix and primers SEQ ID Nos. 76 and 77. The resulting 5958 bp PCR product was purified (Qiaquick gel purification kit, Qiagen) and ligated into pGEMTeasy overnight at 4° C. (Promega). 1 μl of the ligation mix was transformed into Electrocompetent E. coli Genehogs (Invitrogen) by electroporation. Transformed cells were plated on LB-Ampicillin-IPTG-Xgal agar plates and incubated at 37° C. overnight. White colonies were picked into LB-ampicillin broth and incubated at 37° C. overnight with shaking at 220 rpm. Plasmid DNA was isolated by Qiaprep miniprep DNA isolation (Qiagen). NotI digestion of the plasmid DNA indicated whether a 6 kb insert was present and the presence of ILV1352 DNA was confirmed by PCR reactions using the following PCR primer sets: a) SEQ ID Nos. 76 and 77; b) SEQ ID Nos. 78 and 79. Plasmids yielding the following size PCR products were deemed to be pGEMTEasy_ILV 1352: a) 5958 bp, b) 1923 bp.

A plasmid was constructed for transposition of a hygromycin resistance cassette. Firstly, The bacterial zeocin resistance cassette from pEMzeo was introduced into the EcoRI site of pMOD2 between the mosaic ends. Then, the zeocin resistance cassette together with the mosaic ends were amplified by PCR including SpeI sites on the primers. The product was then digested with SpeI and ligated into the SpeI site of pGEMTeasy. The hygromycin resistance cassette was then cloned into the Xba I site. The resulting plasmid (named pPH8) was digested with Spe I and Xmn I to yield a 3649 bp fragment which was gel purified. This fragment (SpeI_PH8) contained mosaic ends for transposition, an Aspergillus hygromycin resistance cassette and a bacterial zeocin resistance marker.

pGEMTEasy_ILV 1352 was mutated by transposition with the EZ::TN transposase kit (Epicentre) using (SpeI_PH8). The following were assembled in a microcentrifuge: 1 μl EZ::TN 10× Reaction Buffer, 1 μl pGEMTEasy_ILV1352, 2 μl (SpeI_PH8), 5 μl sterile water, 1 μl EZ::TN Transposase. The reaction mixture was incubated for 2 hours at 37° C. 1 μl EZ::TN 10× Stop Solution was added, mixed and heated for 10 minutes at 70° C. 1 μl of the stopped reaction was transformed into Electrocompetent E. coli Genehogs (Invitrogen) by electroporation. Transformed cells were plated on LB-Ampicillin-zeocin agar plates and incubated at 37° C. overnight. Colonies were picked into LB-ampicillin broth and incubated at 37° C. overnight with shaking at 220 rpm. Plasmid DNA was isolated by Qiaprep miniprep DNA isolation (Qiagen). Plasmids were screened by PCR using primers SEQ ID Nos. 78 and 80. A PCR product of approximately 900 bp indicated that the transposon PshAI-MB4zeo had inserted approximately 900 bp from the ATG start site of the coding sequence, thus disrupting the gene. The mutant plasmid was designated ILV1352_KO21. The plasmid was digested with DraI and the ˜12 kb fragment was gel purified. This fragment was used for fungal transformation.

### 2.1.3 Fungal Transformation

Initial studies demonstrated that a single knockout of ILV34A was not lethal, but did result in a strain with reduced growth. The effect of knocking out both ILV34A and ILV 1352 was therefore investigated. A brown/white colour diploid pyrG strain of Aspergillus fumigatus (CDP3.1) was transformed with the ILV1352_knockout construct. Transformants were selected on hygromycin and screened by PCR using primers SEQ ID Nos. 80 and 83. Positive clones were checked by Southern blotting to confirm that there was a single knockout. No growth phenotype was observed for the ILV1352 single knockout. The diploid ILV1352 knockout was then transformed with the ILV34A mutant construct and resulting colonies screened by PCR with primers SEQ ID Nos. 80 and 81. Positive clones were checked extensively by PCR and Southern blotting. The diploid was haploidised on benomyl SAB plus uridine and uracil. Haploid spores were assessed for the presence of the hygromycin and pyrG selective markers. No growth was seen when haploid spores were plated on media without uridine and uracil but with hygromycin, indicating that the double knockout was lethal.

### Example 3

### Genomic Sequencing of Genes

The genomic sequences of the genes identified in Example 1 above can be determined experimentally as follows:

### 3.1 Bacterial and Fungal Strains

For bacterial cloning, E. coli Select96 cells (Promega) are used in accordance with manufacturers' instructions.

A. fumigatus clinical isolate AF293 (ref. No. NCPF7367; available to the public from the NCPF repository; Bristol, U.K.); the CBS repository (Belgium) or from Dr. David Denning's clinical isolate culture collection, Hope Hospital, Salford. U.K.) is the preferred strain according to the present invention. AF293 was isolated in 1993 from the lung biopsy of a patient with invasive aspergillosis and aplastic anaemia. It was donated by Shrewsbury PHLS.

3.2 Purification of A. fumigatus Genomic DNA

To obtain mycelial material for genomic DNA isolation, approximately 10⁷ A. fumigatus conidia are inoculated in 50 ml of Vogel's minimal medium and incubated with shaking at 200 rpm until late exponential phase (18-24 h) at 37° C. Mycelium is dried down onto Whatmann 54 paper using a Buchner funnel and a side-arm flask attached to a vacuum pump and washed with PBS/Tween. At this point, the mycelium can be freeze-dried for extraction at a later date.

The mycelium (fresh or freeze dried) is ground to a powder using liquid nitrogen in a mortar cooled to −20° C. The ground biomass is transferred to 50 ml tubes on ice up to the 10 ml mark. An equal volume of extraction buffer (0.7 M NaCl; 0.1 M Na₂SO₃; 0.1 M Tris-HCl pH 7.5; 0.05 M EDTA; 1% (w/v) SDS; pre-warmed to 65° C.) is then added to each tube, mixed thoroughly with a pipette tip and incubated at 65° C. for 20 minutes in a water bath. A volume of chloroform/isoamyl alcohol (24:1) equivalent to the volume of the original biomass is then added to each tube, tubes are mixed thoroughly and incubated on ice for 30 min. Tubes are then centrifuged at 3,500×g for 30 min and the aqueous phase carefully transferred to fresh 50 ml tubes without disturbing the interface.

An equal volume of chloroform/isoamyl alcohol (24:1) is added, the tubes vortexed and incubated on ice for 15 minutes. Tubes are then spun at 3,500×g for 15 minutes. After this spin, if large amounts of precipitate are still present, the supernatant is removed and the chloroform:isoamyl alcohol step repeated. The supernatant is removed and placed in clean sterile Oak Ridge tubes. An equal volume of isopropanol is added and mixed gently. Tubes are incubated at room temperature for at least 15 minutes. Tubes are then centrifuged at 3,030×g for 10 minutes at 4° C. to pellet the DNA. The supernatant is removed and the pellet allowed to air dry for 10-25 minutes. The pellet is suspended in 2 ml sterile water. 1 ml of 7.5 M ammonium acetate is added, mixed and incubated on ice for 1 hour. Tubes are centrifuged at 12,000×g for 30 min, the supernatants transferred to a fresh tube and 0.54 volumes of isopropanol are added, mixed and incubated at room temperature for at least 15 minutes. Tubes are then centrifuged at 5,930×g for 10 min, the supernatant is removed and the pellet washed in 1 ml of 70% ethanol. Tubes are centrifuged at 5,930×g for 10 min and all the ethanol is removed. The pellet is air dried for 20-30 minutes at room temperature and suspended in 0.5-1.0 ml of TE (10 mM Tris-HCl pH 7.5; 1 mM EDTA) Finally, the DNA is treated with RNase A (5 μl of 1 mg/ml stock).

### 3.3 PCR Reactions

Primers pairs are designed to the upstream and downstream regions of the A. fumigatus AF293 genes: The 200-base regions flanking the gene of interest are used as input sequence for Primer3 (http://frodo.wi.mit.edu/cgi-bin/primer3/primer3_www.cgi) to provide a primer pair that spans the gene. If the gene is particularly long it may be necesssary to design primer pairs with internal sequences and thus sequence the gene in parts. The following reagents and conditions are used:

| | | |
|---|---|---|
| 10x high fidelity PCR buffer | 5 | μl |
| dNTP (Clontech: 10 mM) | 1 | μl |
| nH₂O | 39 | μl |
| Pfu Ultra Polmerase (2.5 U/μl) | 1 | μl |
| Primer pairs (10 pmol/μl stock) | 1 | μl each |
| gDNA | | |

PCR cycles are as follows: (1) 95° C., 2 min; (2) 95° C., 30 sec; (3) 54° C., 30 sec; (4) 72° C., 2 min; (5) 72° C., 10 min; (6) 8° C., hold. 40 cycles of steps 2-4 are carried out and the PCR products are run on a gel. The product band is excised from the gel and purified using QIAquick Gel Extraction Kit (Qiagen Ltd, Boundary Court, Gatwick Road, Crawley, West Sussex, RH10 9AX, UK) according to the manufacturers instructions and eluted into 30 μl of sterile water (BDH molecular biology grade/filter sterile).

| | |
|---|---|
| (1:30 dilution of stock) | 2 μl |

### 3.4 Genomic DNA Cloning and Sequencing

Since the gDNA is amplified using Pfu ultra polymerase which produces blunt ends, it is necessary to add ‘A’ overhangs before ligating in to pGEM Teasy. 12.5 μl of purified PCR product is incubated with 12.5 μl 2× PCR Reddy Mix (ABGene) at 70° C. for 30 minutes. The sample is then purified using Qigen Qiaquick gel extraction kit and eluted with 30 μl of molecular biology grade water.

The PCR product is then ligated into pGEM-Teasy (Promega) using the following ligation mixture: 2× Buffer, 5 μl; pGEM Teasy, 1 μl; PCR product, 3 μl; T4 DNA Ligase, 1 μl. The reaction is incubated overnight at 4° C.

2 μl of the ligation mix are then added to Select 96 cells (Promega) and incubated for 20 min on ice. Cells are then heat shocked at 42° C. for 45 sec and placed back on ice. 250 μl of room temp. SOC medium are then added and the cells incubated for 1 hour at 37° C., with shaking at 220 rpm. 50 and 200 μl amounts are then plated on to LB agar plates containing ampicillin (100 μg/ml), 50 μl X-gal (4%) and 10 μl IPTG (100 mM) and incubated over night at 37° C.

Individual white colonies are picked from each transformation are inoculated into LB with ampicillin (100 μg/ml) and incubated overnight at 37° C. with shaking at 220 rpm. Plasmid DNA is extracted using Qiagen miniprep kit according to the manufacturers instructions. 1 μl of plasmid DNA is digested with restriction enzymes for 1 hour at 37° C. Results are compared with the predicted sizes for constructs and clones showing the correct restriction digest pattern are sequenced at MWG Biotech UK Ltd, Waterside House, Peartree Bridge, Milton Keynes, MK6 3BY.

### Example 4

### cDNA Sequencing and RACE

The internal sequences of the genes of interest are experimentally determined by cloning and sequencing cDNA, and the 5′ and 3′ ends of the genes are determined by RACE (Rapid Amplification of cDNA Ends).

4.1 cDNA Cloning and Sequencing
4.1.1 Preparation of A. fumigatus RNA and cDNA

Fungal cultures were prepared as described in Example 3. Cultures were harvested by filtration, then washed twice with DEPC-treated water and transferred to a 50 ml Falcon tube. Samples were frozen in liquid nitrogen and stored at −80° C. until required.

To prepare RNA, fungal samples were ground to a fine powder under liquid nitrogen. RNA was then extracted using the Qiagen RNeasy Plant Mini Kit following the protocol for isolation of total RNA from filamentous fungi in the RNeasy Mini Handbook (06/2001, Pages 75-78, http://www.qiagen.com/literature/handbooks/ma/rnamini/1016272HBRNY_{—}062001WW.pdf). The following modifications were used: At step 3, RLC was used as the lysis buffer of choice; At step 7, the Rneasy column was incubated for 5 min at room temperature after addition of RW1; The optional step 9a was carried out; At step 10, 30 μl RNase-free water was added, the samples incubated for 10 min at room temperature, and then centrifuged; At, step 11, the elution step was repeated to give a total volume of 60 μl RNA.

DNA contamination was removed from the RNA by the addition of Dnase, using 2 μl DNase per μg RNA, in the presence of 10X DNase buffer and incubating at 37° C. for 2 h. DNase-treated RNA was cleaned up using the RNeasy Plant Mini Kit following the RNeasy Mini Protocol for RNA Cleanup (RNeasy Mini Handbook 06/2001, pages 79-81).

To synthesise cDNA from the above RNA the following reaction mixture was prepared: 100 ng-1 μg of DNA-free RNA, 3 μl oligo (dT) (100 ng/μl), and DEPC-treated water to a total volume of 42 μl. Samples were incubated in a heat block at 65° C. for 5 min after which they were allowed to cool slowly to room temperature. Then 2 μl Ultrapure dNTPs, 1 μl reverse transcriptase (Stratascript) and 5 μl 10X reverse transcriptase reaction buffer (Stratascript) were added. Samples were incubated at 42° C. for 1 h, denatured at 90° C. for 5 min and then cooled on ice.

4.1.2 Production of cDNA Constructs

PCR is carried out using the cDNA above to generate cDNA fragments. Primers are designed based on the 5′ and 3′ ends of the predicted genes. PCR reactions are carried out using the following reagents and conditions:

| | | |
|---|---|---|
| 10x high fidelity PCR buffer | 5 | μl |
| dNTP (clontech: 10 mM) | 1 | μl |
| MgSO₄ (50 mM) | 2 | μl |
| nH₂O | 37.8 | μl |
| Platinum TAQ Polmerase (5 U/μl) | 0.2 | μl |
| Primer pairs (10 pmol/μl stock) | 1 | μl each |
| cDNA | 2 | μl |

PCR cycles are run as follows; (1) 94° C., 5 min; (2) 94° C., 30 sec; (3) 53° C., 30 sec; (4) 68° C., 90 sec; (5) 68° C., 10 min; (6) 8° C., pause. Cycles 2-4 are run 40 times. The PCR products are purified using QIAquick PCR Purification Kit (Qiagen Ltd, Boundary Court, Gatwick Road, Crawley, West Sussex, RH10 9AX, UK) according to the manufacturers instructions and run on agarose gels. PCR products are ligated into pGEM-Teasy, used to transform Select 96 cells, and sequenced as described in Example 3 above.

### 4.2 RACE

To determine the 5′ and/or 3′ ends of the genes, RACE (Rapid Amplification of cDNA Ends) was carried out, using the GeneRacer™ Kit (Invitrogen; cat. No. L1502-01), essentially as per manufacturers instructions.

### 4.2.1 Preparation of RNA

A. fumigatus biomass was prepared as described in Example 3. RNA was prepared using the FastRNA kit (QBIOgene) following the manufacturer's instructions (Revision 6030-999-1J05) with the following amendments: At step 1, 40 mg of biomass was used per extraction; At step 2, samples were processed for 20 seconds at speed 5, incubated on ice for 3 minutes, and processed again for 20 seconds at speed 5; At step 3 samples were centrifuged for 5 minutes; At step 5, 500 μl DIPS were added, mixed, and incubated at room temperature for 2 minutes. Samples were mixed again and incubated for a further 2 minutes; At step 6 two washes in 250 μl SEWS were carried out; At step 7, the pellet was disolved in 50 μl SAFE buffer.

### 4.2.2 RACE

1 μg total RNA prepared as described above was de-phosphorylated in a 10 μl reaction using 10 units of calf intestinal phosphate (CIP), 1 μl 10× CIP buffer and 40U RNaseOut™ (made up to 10 μl in DEPC water) at 50° C. for 1 hour. Samples were then made up to 100 μl with DEPC water and the RNA extracted with 100 μl (25:24:1) phenol:chloroform:isoamyl alcohol. RNA was then precipitated by the addition of 2 μl mussel glycogen (10 mg/ml), 10 μl 3M sodium acetate, pH 5.2 and 220 μl 95% ethanol and the sample frozen on dry ice for 10 minutes. RNA was pelleted by centrifugation at 14,500 rpm for 20 minutes at 4° C., washed with 70% ethanol, air dried and re-suspended in 8 μl DEPC water.

De-phosphorylated RNA (7 μl) was de-capped in a 10 μl reaction with 0.5 U tobacco acid pyrophosphatase (TAP), 1 μl 10× TAP buffer and 40 U RnaseOut™ for 1 hour at 37° C. RNA was extracted with phenol:chloroform and precipitated as above, and then re-suspended in 7 μl DEPC-treated water.

De-phosphorylated, de-capped RNA (7 μl) was added to the pre-aliquoted GeneRacer™ RNA Oligo (0.25 μg) and incubated at 65° C. for 5 minutes. A 10 μl ligation reaction is then set up by the addition of 1 μl 10× ligase buffer, 1 μl 10 mM ATP, 40 U RnaseOut™ and 5 U T4 RNA ligase and incubated at 37° C. for 1 hour. RNA was extracted and precipitated as described previously and re-suspended in 11 μl DEPC-treated water.

First-strand cDNA is prepared by the addition of 1 μl GeneRacer™ Oligo dT primer and 1 μl dNTP mix (10 mM each) to 10 μl ligated RNA and incubated at 65° C. for 5 minutes. The following reagents were added to the 12 μl ligated RNA and primer mix; 4 μl 5× first strand buffer, 2 μl 0.1 M DTT, 1 μl RNaseOut™ and 1 μl SuperScript™ II RT (200 U/μ1) and incubated first at 42° C. for 50 minutes and then, to stop the reaction, at 70° C. for 15 minutes. 2 U RNase H was added to the reaction mix and incubated at 37° C. for 20 minutes.

To amplify the 5′ cDNA ends a 50 μl PCR reaction is set up using 1 μl of the RACE-ready cDNA prepared above, 1 μl GeneRacer™ 5′ primer, 1 μl reverse gene-specific primer (designed against the complementary strand of the coding sequence: 5 pmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table V below.

A second, nested PCR stage may also be carried out. This is set up using 1 μl of the RACE cDNA from the first stage above, 1 μl Nested 5′ primer (supplied with kit), 1 μl second reverse gene-specific primer (designed against the complementary strand of the coding sequence and nested with respect to the above primer: 5 μmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table V below.

To amplify 3′ ends a 50 μl PCR reaction is set up using 1 μl of the RACE-ready cDNA prepared above, 1 μl GeneRacer™ 3′ primer (10 μM), 1 μl forward gene-specific primer (designed against the coding strand of the coding sequence: 5 pmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table V below:

A second, nested PCR stage may also be carried out. This is set up using 1 μl of the 3′ RACE cDNA from the first stage above, 1 μl Nested 3′ primer (supplied with kit), 1 μl reverse gene-specific primer (designed against the coding strand of the coding sequence and nested with respect to the above primer: 5 pmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table V below.

5′ and 3′ RACE identify the 5′ ATG and 3′ stop codons as well as giving the 5′ and 3′ untranslated regions of the genes.

TABLE V
Cycling parameters for 5′ and 3′RACE

| 5′ and 3′ RACE | | Nested PCR | |
|---|---|---|---|
| 94° C. | 2 min 1 cycle | 94° C. | 2 min 1 cycle |
| 94° C. | 30 sec 5 cycles | 94° C. | 30 sec 25 cycles |
| 72° C. | 1 min | 67° C. | 30 sec |
| 94° C. | 30 sec 5 cycles | 68° C. | 1 min |
| 70° C. | 1 min | 68° C. | 10 min 1 cycle |
| 94° C. | 30 sec 25 cycles | 8° C. | Hold |
| 64° C. | 30 sec | | |
| 68° C. | 1 min | | |
| 68° C. | 10 min 1 cycle | | |
| 8° C. | Hold | | |

To determine the 5′ end of ILV34 a 50 μl PCR reaction was set up using 1.5 μl of RACE-ready cDNA prepared as described above, 3 μl GeneRacer™ 5′ primer, 1 μl reverse gene-specific primer (designed against the complementary strand of the coding sequence; SEQ ID No. 67: 10 pmol/μl stock), 1 μl dNTP solution (10 mM each), 2 μl 50 mM MgSO₄, 5 μl High Fidelity PCR buffer, 1 μl Platinum® Taq DNA Polymerase High Fidelity (5 U/μl) and 36 μl sterile water. Cycling parameters are given in Table VI below. 5′ RACE confirmed the predicted 5′ start site and first intron of ILV34.

To amplify the 5′ cDNA end of ILV1352 a 50 μl PCR reaction was set up using 1 μl of the RACE-ready cDNA prepared above, 1 μl GeneRacer™ 5′ primer, 1 μl reverse gene-specific primer (designed against the complementary strand of the coding sequence: SEQ ID No. 68; 5 pmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table VI below. A 550 b.p. product was cloned into pCR4-Topo as per manufacturers instructions and sequenced using T7 and T3 sequencing primers. 5′ RACE confirmed the predicted 5′ start site of ILV1352.

TABLE VI
Cycling parameters for 5′ RACE

| ILV34 | ILV1352 |
|---|---|
| 94° C. 2 min, 1 cycle | 94° C. 2 min 1 cycle |
| 94° C. 30 sec, 72° C. 1 min, 4 cycles | 94° C. 30 sec, 72° C. 1 min, 5 cycles |
| 94° C. 30 sec, 70° C. 1 min, 4 cycles | 94° C. 30 sec, 70° C. 1 min, 5 cycles |
| 94° C. 30 sec, 64° C. 30 sec, 68° C. | 94° C. 30 sec, 64° C. 30, sec 68° C. |
| 1 min, 29 cycles | 1 min, 25 cycles |
| 68° C. 10 min, 1 cycle | 68° C. 10 min, 1 cycle |
| 8° C., hold | 8° C. Hold |

### Example 5

### Identification of Fungal Homologs of Genes of Interest

Homologs of the proteins or polynucleotides of the invention can be identified in other fungi by means of bioinformatics analysis. Sequences identified by bioinformatics can be used to design primers which in turn can be used in PCR to generate DNA coding for the homologs. Alternatively, degenerate PCR can be used to obtain sequence, which can then be used to generate probes for screening cDNA or genomic libraries of the organism of interest to identify clones containing the homologs. As a further alternative Southern blots, using fragments of genes from one species as probes, can be used to identify the presence of a homolog in the genome of a second species. The same probe can then be used to screen cDNA or genomic DNA libraries. Once clones corresponding to the novel genes have been identified they can be expressed for functional characterisation of the protein.

### 5.1 Identification of Homologs by Bioinformatics

Homologs of the proteins and polynucleotides of the invention can be identified by searching locally held databases, as detailed in Table VII, using BLAST with SEQ ID Nos: 3, 6, 9, 12, 15, 18, 21, 24, 27, 30, 33, 36, 39, 42, 45, 48, 50, 53, 56, 59, 61 or 63 as the query sequence. Where necessary, matching contigs are down-loaded and genes predicted from genomic DNA as described in Example 1. Alternatively, BLAST searches can be carried out over the web.

TABLE VII
Sources of data for local BLAST searches

| | | BLAST | |
|---|---|---|---|
| Organism | Sequence | flavour | Source |
| Aspergillus fumigatus | Genome | tblastn | www.sanger.ac.uk |
| Candida albicans | ORFs | tblastn | www- |
| | | | sequence.stanford.edu/group/candida/ |
| Cryptococcus neoformans | cDNA | tblastn | http://www.genome.ou.edu/cneo.html |
| Fusarium graminearum | Proteins | blastp | ww.broad.mit.edu |
| Magnaporthe grisea | Proteins | blastp | www.broad.mit.edu |
| Neurospora crassa | Proteins | blastp | www.broad.mit.edu |
| Schizosaccharomyces pombe | Proteins | blastp | nr database ftp://ftp.ncbi.nih.gov/blast/ |
| and Saccharomyces cerevisiae | | | |
| Ustilago maydis | Genome | tblastn | www.broad.mit.edu |
| Fungal pathogen ESTs¹ | ESTs | tblastn | http://cogeme.ex.ac.uk/blast.html |

¹This dataset contains ESTs from the following plant pathogen fungi: Blumeria graminis, Botryotinia, Cladosporium fulvum, Colletotrichum trifolii, Cryphonectria parasitica, Fusarium sporotrichioides, Gibberella zeae, Leptosphaeria maculans, Magnaporthe grisea, Mycosphaerella graminicola, Phytophthora infestans, Phytophthora sojae, Sclerotinia sclerotiorum, Ustilago maydis and Verticillium dahliae.

The relationships between SEQ ID Nos: 3, 6, 9, 12, 15, 18, 21, 24, 27, 30, 33, 36, 39, 42, 45, 48, 50, 53, 56, 59, 61 or 63, and hits identified from blast searches above can be clarified by phylogenetic analysis, for example using the PHYLIP suite of programs (Felsenstein, Felsenstein, J., 2002. PHYLIP (Phylogeny Inference Package) version 3.6a3. Distributed by the author. Department of Genome Sciences, University of Washington, Seattle). A distance matrix is generated using PROTDIST with the Jones-Taylor-Thornton model and a tree inferred using FITCH with global rearrangements and 10 jumbles of input order. 100 bootstrap replicates are generated using SEQBOOT, distance matrices generated using PROTDIST as above, trees inferred using NEIGHBOUR, and then bootstrap values and the consensus trees are calculated using CONSENSE. Trees are viewed using TREEVIEW (Page, 1996 Page, R. D. M., 1996. TREEVIEW: An application to display phylogenetic trees on personal computers. Computer Applications in the Biosciences 12, 357-358.). Preliminary phylogenetic trees can be generated “on the fly” by the multiple alignment package QAlign (Sameth et al., 2003, Bioinformatics 19, 1592-1593; http://www.ridom.de/qalign).

Alternatively, the relationship between SEQ ID Nos: 3, 6, 9, 12, 15, 18, 21, 24, 27, 30, 33 36, 39, 42, 45, 48, 50, 53, 56, 59, 61 or 63 and homologs can be clarified using reciprocal blast hits as described by e.g., Wall et al. (Bioinformatics 19, 1710-1711).

ILV3 sequences in filamentous fungi other than A. nidulans and A. fumigatus were identified by means of the methods described above and by BLAST searches against the NCBI nr database. Protein sequences were aligned with Aspergillus ILV3 proteins and gene predictions improved where necessary. The resulting sequences (SEQ ID Nos 37-63) are summarised in Table II. From the alignment, it was possible to cluster the ILV3 sequences into two groups of orthologs, indicated in the table as group I, clustering with A. fumigatus sequence SEQ ID No. 21, and group II, clustering with A. fumigatus SEQ ID No. 12.

### 5.2 Identification of Homologs by Degenerate PCR

5.2.1. Preparation of Genomic DNA from Organism of Interest

Fungal cultures are prepared using methods suitable, for particular species. For example, Aspergillus and Candida species, Cryptococcus neoformans, Fusarium solani and Trichophyton species are maintained on Sabouraud dextrose agar at 30-35° C.; Leptosphaeria nodorum on Malt agar medium (30 g/L malt extract; 15 g/L Bacto-agar, pH 5.5), 24.0° C.; Magnaporthe grisea on oatmeal agar (6.7 g/L agar, 53.3 g/L instant oatmeal) 25.0° C., or Cornmeal agar (Difco 0386), 26.0° C.; Phytophthora capsici cultures are maintained on V-8 agar at 24° C.; Pyricularia oryzae cultures are maintained on rice polish agar at 24° C. under white fluorescent lights (12 hr artificial day), and are subcultured every 7-14 days by the transfer of mycelial plugs to fresh plates; Pythium ultimum cultures are maintained on PDA at 24° C., and subcultured every 7 days by the transfer of aerial mycelium to fresh plates with an inoculating needle; Rhizoctonia solani cultures are maintained on PDA at 24° C. under fluorescent lights (12 h artificial day), and subcultured every 7 days by the transfer of mycelial plugs to fresh plates; Ustilago maydis cultures are maintained on PDY agar at 30° C. in the dark, and subcultured by re-streaking. Genomic DNA is prepared from cultures using standard methodologies, e.g. using the Qiagen DNeasy Plant Kit, or using methods described in Example 3.

### 5.2.2 PCR

Primers are designed to correspond to regions conserved between the gene of interest and its homologs (identified as described above). Those skilled in the art will appreciate that it may be necessary to try a range of primer pairs. PCR reactions using the primer pairs are set up as follows:

| | |
|---|---|
| 2x ReddyMix PCR mastermix (ABgene) | 12.5 μl |
| Primers (5 pmol) | 1 μl each |
| template gDNA | 1.5-4 μg/ml |
| nuclease-free water | to final volume of 25 μl |

The reactions are run using the following conditions on a Biometra personal PCR cycler (Thistle Scientific Ltd, DFDS House, Goldie Road, Uddington, Glasgow, G71 6NZ): (1) 95° C., 5 min; (2) 95° C., 1 min; (3) 53° C., 1 min 30 sec; (4) 68° C., 2 min 30 sec; (5) 72° C., 10 min; (6) 4° C., Hold. 30 cycles of steps 2-4 are carried out. The PCR products are purified (to remove residual enzymes and nucleotides) using Qiagen's QIAquick PCR Purification Kit (Qiagen Ltd, Boundary Court, Gatwick Road, Crawley, West Sussex, RH10 9AX, UK) according to the manufacturers instructions and eluted into 40 μl of sterile water (BDH molecular biology grade/filter sterile). The purified PCR products are examined on 1% agarose gels. Those skilled in the art will appreciate that degenerate PCR may require variations in a number of parameters in the attempt to generate a product. These include primer concentration, template concentration, concentration of Mg²⁺ ions, elongation and annealing times, and annealing temperature. Variations in temperature can be accommodated by the use of a gradient PCR machine.

The purified PCR products are cloned into pPEM-Teasy (Promega) and then transformed into XL10-Gold® Kan ultracompetent E. coli cells according to the manufacturers instructions. The transformation reactions are then plated onto LB agar plates containing ampicillin (100 μg/ml), 50 μl X-gal (4%) and 10 μl IPTG (100 mM). Following overnight incubation at 37° C., individual white colonies from each transformation are sub-cultured into LB broth containing ampicillin (100 μg/ml). After overnight incubation at 37° C. with shaking, plasmids are extracted using Qiagen spin mini plasmid extraction kits according to the manufacturers instructions and sent away for full-length sequencing.

### 5.3 Identification of Homologs by Southern Blotting

### 5.3.1 Digestion of Genomic DNA and Transfer to Nylon Membranes

Genomic DNA from the fungi of interest are digested with the appropriate restriction enzyme and run on 0.8% agarose gel. The gel is then submerged in 250 mM HCl for no more than 10 mins, with shaking, at room temperature, after which the gel is rinsed with sterilised RO water.

Transfer of the DNA onto nylon membrane is carried out using 0.4 M NaOH. Transfer protocols and apparatus are well known and are described in e.g. Sambrook et al., (1989), Molecular Cloning, 2^{nd} Edition., Cold Spring Harbor Laboratory Press. After transfer, the DNA is fixed to the membrane by baking at 120° C. for 30 min. The membrane can then be used immediately, or stored dry for future use.

### 5.3.2. Preparation of Probe

Probes are generated either by restriction digests of DNA or by PCR of an appropriate region. A suitable probe can be generated by PCR using a primer pair designed using Primer3 (http://frodo.wi.mit.edu/cgi-bin/primer3/primer3_www.cgi) and A. fumigatus genomic DNA.

1 μg DNA template is diluted in molecular biology water to a total volume of 16 denatured in a boiling water bath for 10 mins, and quickly chilled on ice. 4 μl DIG-High Prime (1 mM dATP, 1 mM dCTP, 1 mM dGTP, 0.65 mM dTTP, 0.35 mM alkali-labile-digoxygenin-11-dUTP, 1 U/μl labelling grade Klenow enzyme, 5× reaction buffer, in 50% (v/v) glycerol) is then added and the reaction incubated at 37° C. for 20 hours, after which 2 μl of 200 mM EDTA pH 8.0 is added to terminate the labelling reaction. The labelling efficiency is estimated by comparison with DIG-labelled control DNA.

### 5.3.3.Prehybridisation and Hybridisation

The membrane is placed in a hybridisation tube containing 20 ml of prehybridisation solution (DIG Easy Hyb, Roche) per 100 cm² of membrane surface area and prehybridised at 42° C. for 2 hours in a hybridisation oven. The DIG-labelled probe is denatured by heating in a boiling water bath for 10 min and then chilled directly on ice. The probe is then diluted to ˜200 ng/mL in hybridisation solution (Easy Hyb, Roche; at least 5 mL of hybridisation solution is required per hybridisation). The prehybridisation solution is discarded from the hybridization tube and the hybridisation solution containing the DIG-labelled probe added quickly. The hybridisation then proceeds overnight at a 42° C. in the hybridisation oven. The optimum temperature is dependant on probe size and homology with target sequence and is determined empirically.

After hybridisation, the membrane is washed twice at 42° C., 5 mins per wash, with 50 mL of stringency wash solution (3×SSC, 0.1% SDS; where 20×SSC buffer is 3 M NaCl, 300 mM sodium citrate, pH 7.0), followed by two washes at RT, 15 min per wash, in 50 mL stringency wash solution. The stringency of these washes can be decreased by increasing the SSC concentration to 6×SSC, 0.1% SDS and/or decreasing the wash temperatures.

### 5.3.4. Detection

The membrane is washed in 20 mL washing buffer (100 mM maleic acid, 150 mM NaCl; pH 7.5; 0.3% v/v Tween 20), and then incubated successively with the following; 20 mL blocking solution (1% w/v blocking reagent for nucleic acid hybridisation, Roche, dissolved in 100 mM maleic acid, 150 mM NaCl, pH 7), for 30 min at room temperature; Anti-DIG-alkaline phosphatase (Roche) diluted 1:5,000 in blocking buffer, 30 min at room temperature; Washing buffer, two washes each of 15 min at room temperature; Detection buffer (100 mM Tris-HCl, 100 mM NaCl; pH 9.5), 2 min at room temperature. The membrane is then removed, placed on top of an acetate sheet, and ˜0.5 ml (per 100 cm²) of CSPD or CDP-star added to the top of the membrane. A second sheet of acetate is then placed over the surface of the membrane, the assembly incubated for 5 min at room temperature and then sealed in a plastic bag. The assembly is then exposed to X-ray film for between 15 min and 1 hour. Optimal exposure time is determined empirically by increasing exposure time up to 24 hours.

The presence of a band on the gel is evidence of a gene in the genomic DNA of interest. The molecular weight of the band depends on the size of the restriction fragment that contains the gene.

### Example 6

### Expression of Recombinant Proteins and/or Fragments

Recombinant proteins or fragments are expressed to enable detailed study of function and for the development of an in vitro high-throughput screen for inhibitory compounds. PCR is carried out using cDNA, prepared as described above, to generate polynucleotides encoding protein sequence essentially corresponding to SEQ ID Nos. 3, 6, 9, 12, 15, 18, 21, 24, 27, 30, 33, 36, 39, 42, 45, 48, 50, 53, 56, 59, 61 or 63.

Primers are designed to encode the 5′ and 3′ ends of the coding sequences, with the addition of bases necessary to anneal with the pET-30 Xa/LIC vector (5′ additional sequence, GGTATTGAGGGTCGC; 3′ additional sequence, AGAGGAGAGTTAGAGCC). If the protein has an N-terminal leader peptide, this should be excluded. If the protein is made up of multiple domains, it may be desirable or necessary to express only a limited number of domains, or even a single domain. In these cases, primers are designed to correspond to domain boundaries. PCR reactions are carried out using the following reaction mixture and conditions. All Reagents are present in the KOD kit (Novagen).
2.5 μl 10× PCR Buffer
5 μl dNTPs (2 mM)
2 μl MgSO₄ (25 mM)
1 μl each primer (5 pmol each)
1 μl template cDNA
11.5 μl nuclease-free water
1 μl KOD Polymerase
   PCR reactions are run using the following conditions: (1) 94° C., 5 min; (2) 94° C., 1 min; (3) 59.3° C., 1 min; (4) 68° C., 1 min 30 sec; (5) 68° C., 10 min; (6) 10° C., hold. 40 cycles of steps 2-4 are carried out and the PCR products purified using QIAquick PCR Purification Kit (Qiagen Ltd, Boundary Court, Gatwick Road, Crawley, West Sussex, RH10 9AX, UK) according to the manufacturers instructions. The purified PCR products are examined on agarose gels.

cDNA fragments are then cloned in to the pET30 Xa/LIC vector (Novagen), transformed into Nova Blue chemically competent E. coli cells, and plated on to a prewarmed kanamycin (+) selection plate. After an overnight incubation at 37° C., kanamycin-resistant colonies are selected and grown up in kanamycin containing LB medium. Plasmid DNA is isolated using the Plasmid Mini Kit (Qiagen). Confirmation of the presence and correct orientation of the inserts is determined by restriction analysis and sequencing of the construct.

Purified plasmid DNA, which is been confirmed to be of the correct sequence and orientation, is transformed into chemically competent BL21 Star (DE3) One Shot E. coli cells and grown overnight at 37° C. 2 ml of an over-night culture are used to innoculate 100 ml of LB, 30 μg/ml kanamycin, and the cultures incubated at 37° C., 220 rpm until the cell density reaches an optical density of 0.6 (approximately 3 hours). Expression of the recombinant protein is then induced with IPTG (1 mM) for 5 hours.

Bacteria are harvested by centrifugation at 4500 rpm for 10 minutes and the pellets lysed in lysis buffer (10 ml Bugbuster (Novagen), 10 μl Benzonase (Novagen), 0.4 μl lysozyme (Novagen) and 100 μl 1M imadazole for 20 minutes at room temperature. Cells are then spun down at 16000 g for 20′ at 4° C. and the supernatant, containing soluble recombinant protein, removed to a clean tube.

Supernatant is added to prewashed Ni-Nta resin at a concentration of 5-10 mg protein per ml of resin and allowed to bind for 1 hour at 4° C. Protein-resin mix is then poured into a column, washed twice in 4 ml of wash buffer (2.5 ml 1M phosphate buffer pH8, 6.25 ml 4M NaCl, 1 ml 1M Imidazole pH8, 0.5 ml 10% Tween 20; made up to 50 mls in n.H₂O) and then eluted in 4×0.5 ml fractions with elution buffer (250 μl 1M Phosphate Buffer pH8, 625 μl 4M NaCl, 1.25 ml 1M Imidazole pH8, 50 μl 10% Tween 20, Made up to 5 mls in n.H₂O). Fractions containing purified protein are identified by SDS-Page and Western blotting using an S-tag HRP conjugate (Novagen), pooled and then desalted using a PD10 column (Amersham) equilibrated with 25 ml of 0.1 M KPO₄ pH7. Fractions containing purified recombinant protein can be concentrated using YM10 columns (Millipore) and stored at −80° C.

Alternative expression systems can be used for expression in bacteria, such as the glutathione S-transferase or mannose-binding fusion-protein system.

### 6.1 Expression of Recombinant ILV34

A full-length and a truncated version of ILV34 (Table II) were expressed. The truncated version lacked the 29 amino acid N-terminal mitochondrial targeting sequence. Primers were designed to encode the 5′ and 3′ ends of the coding sequence, with the addition of bases necessary to anneal with the pET-30 Ek/LIC vector (full length construct 5′ primer SEQ ID No. 64; truncated construct 5′ primer SEQ ID No. 65; common 3′ primer, SEQ ID No. 66). PCRs were carried out using the following reaction mixture and conditions (all reagents were present in the KOD Hot Start DNA Polymerase kit, Novagen):

5 μl 10× PCR buffer; 5 μl dNTPs (2 mM); 3 μl MgSO₄ (25 mM); 1.5 μl each primer (15 pmol each); 1 μl template cDNA; 29.5 μl nuclease-free water; 2.5 μl DMSO; 1 μl KOD Hot Start polymerase

PCRs wre run using the following conditions: (1) 94° C., 5 min; (2) 94° C., 1 min; (3) 59° C., 1 min 30 sec; (4) 68° C., 1 min 30 sec; (5) 68° C., 10 mM; (6) 8° C., hold. 40 cycles of steps 2-4 were carried out and the PCR products purified using QIAquick PCR Purification Kit (Qiagen Ltd, Boundary Court, Gatwick Road, Crawley, West Sussex, RH10 9AX, UK) according to the manufacturers instructions. The purified PCR products were examined on agarose gels.

cDNA fragments were then cloned into the pET30 Ek/LIC vector (Novagen), transformed into Nova Blue chemically competent E. coli cells, and plated on to a pre-warmed kanamycin (+) selection plate. After an overnight incubation at 37° C., kanamycin-resistant colonies were selected and grown up in kanamycin-containing LB medium (30 μl/ml). Plasmid DNA was isolated using the Plasmid Mini Kit (Qiagen). Confirmation of the presence and correct sequence and orientation of the inserts was determined by restriction analysis, PCRs and sequencing of the construct.

Purified plasmid DNA with of the correct sequence and orientation was transformed into chemically competent BL21 Star (DE3) One Shot E. coli cells and grown overnight at 37° C. 6 ml of an overnight culture were used to inoculate 200 ml of LB, 30 μg/ml kanamycin, and the cultures incubated at 37° C., 220 rpm until the cell density reached an optical density of 0.5-0.7 (approximately 2 hours). Expression of the recombinant protein was then induced with IPTG (0.5 mM) for 20 hours at 20° C. Bacteria were harvested by centrifugation at 3500 g for 10 minutes and the pellets lysed in lysis buffer (24 ml Bugbuster, Novagen; 24 μl Benzonase, Novagen; 0.4 μl rLysozyme, Novagen; and 1200 0 μM imidazole) for 20 minutes with mixing at room temperature. Cell debris was then removed by centrifuging the sample at 16000 g for 20 minutes at 4° C. and the supernatant, containing soluble protein, removed to a clean tube.

Supernatant was added to pre-washed Ni-NTA resin at a concentration of approximately 25 mg protein per ml of resin and allowed to bind for 1 hour at 4° C. with mixing. Protein/resin mix was then poured into a large disposable plastic column, washed twice in 7.5 ml wash/bind buffer (2.5 ml 1M Na₂HPO₄ pH8.0, 6.25 ml 4 M NaCl, 1 ml 1 M imidazole pH8.0, 0.5 ml 10% Tween 20, made up to 50 ml with dH₂O) and then eluted in 6.5 ml elution buffer (1 ml 1 M Na₂HPO₄, pH8.0, 2.5 ml 4 M NaCl, 5 ml 1 M imidazole pH8.0, 200p. 1 10% Tween 20, 200 μl protease inhibitor cocktail III, made up to 20 ml with dH₂O). The presence of purified ILV34 protein in the eluate was confirmed by SDS-PAGE, and the eluate was then desalted using PD10 columns equilibrated with buffer containing 50 mM Tris-HCl, 10 mM MgCl₂, pH 8.0. Aliquots were stored at −80° C.

### 6.2 Expression of Recombinant ILV1352

Constructs encoding full-length and truncated versions of ILV1352 (Table II) were produced. The truncated version lacks the first 84 base pairs of the ILV1352 DNA sequence. Primers were designed to encode the 5′ and 3′ ends of the coding sequence, with the addition of bases necessary to anneal with the pET-30 Ek/LIC vector; (5′ primer, full length, SEQ ID No. 68; 5′ primer truncated, SEQ ID No. 69; common 3′ primer, SEQ ID No. 70). PCRs were carried out using the following reaction mixture and conditions. All reagents were present in the KOD Hot Start DNA Polymerase kit (Novagen); 2.5 μl 10× PCR buffer, 2.5 μl dNTPs (2 mM), 1 μl MgSO₄ (25 mM), 1.5 μl each primer (5 pmol/μl), 1 μl template cDNA, 15 μl nuclease-free water, 0.5 μl KOD Hot Start polymerase.

PCRs were run using the following conditions: (1) 95° C., 5 min; (2) 95° C., 1 min; (3) 56° C., 1 mM 30 sec; (4) 68° C., 2 min 30 sec; (5) 68° C., 10 min; (6) 8° C., hold. 45 cycles of steps 2-4 were carried out and the PCR products purified using QIAquick PCR Purification Kit (Qiagen Ltd, Boundary Court, Gatwick Road, Crawley, West Sussex, RH10 9AX, UK) according to the manufacturers instructions. The purified PCR products were examined on agarose gels.

cDNA fragments were then cloned into the pET30 Ek/LIC vector (Novagen), transformed into Nova Blue chemically competent E. coli cells, and plated on to a pre-warmed kanamycin (+) selection plate. After an overnight incubation at 37° C., a kanamycin-resistant colony was selected and grown up in kanamycin-containing LB medium (30 μg/ml). A glycerol stock was produced from the culture, the remains of which were used to purify plasmid DNA using Qiagen's Plasmid Mini Kit. Confirmation of the presence and correct sequence and orientation of the inserts was determined by PCR and sequencing of the construct. Purified plasmid DNA was transformed into chemically competent BL21 Star (DE3) One Shot E. coli cells.

Preliminary studies showed that recombinant truncated ILV1352 accumulated in inclusion bodies. The inclusion bodies were purified and truncated ILV1352 solubilised and re-folded as follows: The glycerol stock, produced from BL21 cells containing truncated ILV1352 in pET30 Ek/LIC, was used to inoculate 10 ml LB, 30 μg/ml kanamycin broth. The broth was incubated overnight at 37° C., with shaking at 220 rpm. The culture was added to 90 ml LB kanamycin broth and incubated at 37° C., until the OD₆₀₀ had reached between 0.4-1.0 (approximately 1.5 hr). At this point, IPTG (0.1 mM) was added to the culture which was incubated at 30° C. for 5 hr. Cells were harvested by centrifugation at 8,500 rpm for 10 min. Pellets were resuspended in Bugbuster Master Mix (5 ml per 100 ml culture) and incubated at room temperature for 20 min with shaking.

The cell suspension was centrifuged at 11,000 rpm for 20 min at 4° C. After removal of the supernatant, the pellet was resuspended in 5 ml Bugbuster Master Mix. Six volumes (30 ml) 1:10 Bugbuster Protein Extraction Reagent was added to the cell suspension and inclusion bodies were collected by centrifugation at 6,000 rpm for 15 min at 4° C.

The supernatant was removed and inclusion bodies were resuspended in 50 ml 1:10 Bugbuster reagent. The cell suspension was centrifuged at 6,000 rpm for 15 min at 4° C. This wash step was repeated two further times, but in the final step centrifugation speed was increased to 11,000 rpm. The final pellet of purified inclusion bodies was resuspended in a 0.1 culture volume (10 ml) of 1×IB Wash Buffer (Novagen). Inclusion bodies were collected by centrifugation at 8,500 rpm for 10 min. The pellet was resuspended in 0.1 culture volume of 1×IB Wash Buffer and inclusion bodies were collected by centrifugation at 8,500 rpm for 10 min. The supernatant was removed and the inclusion bodies were resuspended in 1×IB Solubilisation Buffer plus 0.3% N-lauroylsarcosine and 1 mM DTT (Novagen) at a concentration of 10 mg/ml. The sample was mixed gently, incubated for 15 min at room temperature and centrifuged at 8,500 rpm for 10 min. The solubilised fraction of ILV1352 was dialyzed against three changes of neutral pH buffer (20 mM Tris-HCl, pH 8.5)+0.1 mM DTT using a Slide-A-Lyzer 7K MWCO dialysis cassette (Pierce), with DTT omitted from the final dialysis step. Example 7. Assays for the identification of inhibitors

### 7.1 Biochemical Assays for the Identification of Inhibitors

Recombinant proteins can be assayed using an assay type specific for the particular protein. For example:

Endonucleases can be asayed by incubating the protein with DNA, such as Lamdba or pBR322, and observing whether the DNA is cleaved by running on an agrose gel.

Exonucleases can be assayed by incubating the protein with fluorescently or radio-labelled DNA, such as Lamdba or pBR322, and observing whether the fluorescent or labelled nucleotides are released.

Exoribonucleases can be assayed by incubating the protein with fluorescent or radiolabelled RNA and observing whether fluorescent or labeled ribonucleotides are released.

GPCRs (G-protein coupled receptors) can be assayed by incubating radiolabelled ligand with GPCR membrane fractions derivatised with FlashBlue™ beads (Perkin Elmer) and measuring emitted light. GPCR membrane fractions are prepared from cells expressing, or over-expressing the GPCR of interest.

ILV3/ILV3/dihydroxyacid dehydratases can be assayed by measuring the formation of the keto acid reaction products, either directly, at 313 nm, or by derivatising the ketone with 2,4-dinitrophenyl hydrazine and measuring at 530 nm.

Kinases can be assayed by incubating the kinase with [³²P]-ATP and substrate, and measuring the incorporation of ³²P-label into the substrate. Suitable substrates may include myelin basic protein, glycogen synthase and enolase. Alternatively, fluorescence queueing technology such as QTL Lightspeed™ kinase assays (QTL biosystems, Reigate, Surrey) can be used.

Phosphatases can be assayed by incubating the protein with [³²P]-ATP-labelled substrate and measuring the release of ³²P-label. Suitable substrates may include myelin basic protein, glycogen synthase and enolase. Alternatively, phosphatase assays exploiting fluorescence quenching technology, such as IQ phosphatase assays (Pierce, Cramlington, Northumberland) can be used.

Phosphatididylinositol-specific phospholipase Cs can be assayed by using the chromogenic substrate 5-bromo-4-chloro-3-indoxyl-myoinositol-1-phosphate or the fluorogenic substrate 4-methylumbelliferyl-myo-inositol-1-phosphate (Restaino et al., 1999, J. Food Prot. 62, 244-251; Reissbrodt, 2004, Int. J. Food Microbiol. 15, 1-9).

Phosphodiesterases such as 3′5′ cyclic nucleotide phosphodiesterases can be assayed as described by Wera et al. (FEBS Lett. 1997, 420, 147-150) by following the time-dependent degradation of cAMP. Samples and controls are incubated in 50 mM Tris-HCl (pH 8), 0.1 mMEDTA, and 500 mM cAMP at 30° C. The reaction is stopped by heating, and cAMP is measured using the cAMP [³H] assay system (Amersham, Arlington Heights, Ill.).

Protein tyrosine phosphatases can be assayed using substrate protein (such as myelin basic protein) where the tyrosines have been labelled with [³²P], and measuring released label after incubation with the enzyme. Alternatively, the non-radioactive ProFluor™ assay kit (Promega) can be used.

These assays are modified for the identification of an inhibitor by including a candidate substance in the incubation and measuring the extent to which the enzyme activity is inhibited.

### 7.2 Genetic Screen for the Identification of Inhibitors

In the case of proteins for which a function is not known or obvious, inhibitors can be identified using a generic genetic screen. Heterozygous knock-out mutants are generated, for instance as described in Example 2. In most this should result in less gene product being made by the heterozygote than the wild type diploid. If the gene is essential for growth then the heterozygote should be more sensitive to a compound that targets the product of that gene. This phenomenon is called haploinsufficiency and has been demonstrated in yeast (Genomic profiling of drug sensitivities via induced haploinsufficiency. Giaever G, Shoemaker D D, Jones T W, Liang H, Winzeler E A, Astromoff A, Davis R W. Nat. Genet. 1999 21:278-83.)

The primary screen for genes of unknown function involves monitoring the growth of the heterozygous mutant versus the growth of the wild type diploid strain of Aspergillus fumigatus, in the presence and absence of a panel of compounds. Spore suspensions of these strains are set up in RPMI 1640 medium in 96-well plates. 1×10⁴ cfu/ml is the inoculum used. Potential inhibitors are added to give a final concentration of 32 μg/ml. The plates are then incubated at 37° C. for 48 h. The OD485 of the cultures is then measured using a plate reading spectrophotometer.

If both heretozygote and wild-type are unaffected no further work is carried out on the compound. If there is (a) growth of the wild type but no growth of the heterozygote, or (b) no growth of both strains, the Minimal Inhibitory Concentration (MIC) for the compound in each strain is determined as follows:

The heterozygote mutant and the wild type diploid are incubated in the presence of a range of concentrations of the chemical. The lowest concentration of chemical that prevents growth of the organism (the Minimal Inhibitory Concentration, MIC) is calculated for both strains. Doubling dilutions of the compound of interest are prepared in RPMI 1640 medium in 96-well plates starting at 50 μg/ml down to 0.1 μg/ml in duplicate. Each well is inoculated with either wild type or mutant Aspergillus fumigatus and the plate incubated at 37° C. for 24/48 h prior to measuring the OD485.

An inhibitor of the product of the gene of unknown function will have a lower MIC in the mutant strain than in the wild type strain, i.e., a 2-fold or more difference in MIC between the 2 strains. This anti-fungal compound can then be used as the basis for chemistry approaches to improve the specificity, potency and other properties of the compound.

### 7.3 ILV3 Assay

The assay for ILV34 is based upon the ability of this enzyme to dehydrate dihydroxyacid substrates to a keto acid. The natural substrates are 2,3-dihydroxy-3-methylbutyrate and 2,3-dihydroxy-3-ethylbutyrate; an alternative substrate which is commercially available is L-threonic acid. The appearance of the keto acid product can be monitored directly at 240 nm; alternatively it can be reacted with semicarbazide and sodium acetate and monitored at 250 nm. The semicarbazide/sodium acetate effectively stops the enzymatic reaction and develops it giving an increased absorbance, which is stable for at least 24 hours (Kanamori and Wixom, 1963, J. Biol. Chem. 238:998-1005; Kiritani and Wagner, 1970, Meth. Enzymol. 17:755-764; Limberg et al., 1995, Bioorg. Med. Chem. 3:487-494).

Assays were carried out in 96- or 384-well plates. To each well of a 384-well plate was added 0-8000 ng recombinant truncated ILV34 and 25 μl 0-50 mM threonate (dissolved in 50 mM Tris-HCl, 10 mM MgCl₂, pH8.0), and the volume made up to 50 μl with 50 mM Tris-HCl, 10 mM MgCl₂ (pH8.0). Samples were incubated at room temperature and at suitable intervals the reaction was stopped and developed by the addition of 25 p. 1 semicarbazide solution (1.26% w/v semicarbazide in 1.89% w/v sodium acetate solution). The samples were incubated for 15 mins after the final semicarbazide/sodium acetate addition and then read at 250 nm.

Rate of reaction (change in absorbance per minute) was linear over different ILV34 concentrations but became saturated at high substrate concentrations. ILV34 had a Km of approximately 10 mM for threonate, and was most active at pH 8.0. Magnesium ion concentration had no effect on ILV34 activity in the range 50 μM-10 mM. An inhibitor of ILV34, 2-hydroxy-3-methylbutyric acid (Sigma 219835), was tested and the IC₅₀ found to be approximately 10 mM.

### 7.4 High-Throughput Screen for the Identification of ILV34 Inhibitors

Screens for inhibitors of ILV34 were based on the assay described above. The screen described is for a 384 format but the protocol can be adapted to run 1536 or other formats as required.

Compounds to be tested were dissolved in 100% DMSO, diluted in water and loaded into 384 square well polystyrene plates (eg. ‘Greiner bio-one’ UV-Star 384 Microplates; 10 μl/well). The final DMSO concentration in all assay wells was 5% v/v.

The substrate, L-threonic acid (hemicalcium salt [Aldrich 380644-5G]; 20 mM in 62.5 mM Tris-HCl, 12 mM MgCl₂ pH8.0) was prepared prior to use on the day of the screen. The solution was sonicated at room temperature until clear, a glass rod was used to crush material which was slow to dissolve. The final concentration of L-threonic acid in the assay wells was 8 mM.

The stop/signal amplification reagent (semicarbazide HCl [Aldrich S220-1]; sodium acetate, anhydrous [BDH 301045M]); 1.26% w/v semicarbazide, 1.89% w/v sodium acetate in deionised water) was also prepared prior to use on the day of the screen.

Recombinant ILV34 enzyme prepared as described above was made up in 62.5 mM Tris-HCl, 12 mM MgCl₂ buffer (pH8.0). The final buffer concentration in the assay was 50 mM Tris-HCl, 9.6 mM MgCl₂ buffer (pH8.0).

Assays were carried out using Tecan Freedom, Tecan TeMo and PerkinElmer Minitrak robots together with a ThermoLabsystems multidrop 384 and a Tecan Safire automated plate reader.

20 μl of enzyme (typically around 2 μg/well, depending on specific activity of the batch) followed by 20 μL-threonic acid solution were added to wells of the microtitre plates containing test compounds. 20 μl of 62.5 mM Tris-HCl, 12 mM MgCl₂ buffer (pH8.0) was used for a duplicate set of plates (i.e. for background no-enzyme controls); DMSO (diluted in the same way as solubilised compound stocks) was used for no-compound controls. Plates were incubated at room temperature for 40 minutes after which 25 μl of stop/amplification reagent was added. After 15 minutes at room temperature plates were read at 250 nm and data processed using Excel spreadsheets to convert raw data into percent inhibition data.

The kinetics of the screen over the incubation time were such that reaction progress curves were both linear with time and protein concentration. The Z′ value for the screen was equal to 0.83 and thus fully acceptable (Zhang et al., 1999, J. Biomolecular Screening, 4, 67-73). Consistency of signal between wells on plates, plate to plate, and screen run to screen run were also acceptable for an HTS regime.

Secondary screens can be carried out to measure dose response data for selected compounds, using essentially the same protocol as the pimary screen. The secondary screen uses the Excelfit version 3 software (IDBS), with sigmoidal model 606, to plot appropriate inhibition values and determine IC50 data for compounds.

ILV1352 can be assayed using a similar assay to that employed for ILV34, and ILV1352 inhibitors are identified in a similar way to ILV34 inhibitors. Compounds identified as inhibitors from the ILV34 assay can be tested in a similar assay using recombinant ILV1352 (or vice versa) and compounds showing inhibition in both assays are candidates for antifungal agents. Alternatively, compounds showing inhibition of one of the ILV3 proteins may be ILV3 inhibitors.

### Example 8

### Production of an Antibody

Recombinant protein may be used as an immunogen, (as described in Example 6). Alternatively, synthetic proteins or polypeptides encoding regions either unique to the individual proteins, or likely to provide cross-reactivity within a set of homologs are used. Peptides may need to be conjugated to carrier proteins before immunization.

Preimmune sera from animals to be immunised are screened against the immunogen to ensure that there is no endogenous cross reactivity. Animals (typically sheep, rabbits or mice) are then immunised. For polyclonal antibody production, the resulting sera is affinity purified using the immunogen cross-linked to a chromatography matrix. Alternatively, purification of the antibody fraction from the serum, e.g. using protein G or protein A cross-linked to a matrix, may be sufficient. Monoclonal antibody production proceeds by methods familiar to those skilled in the art.

The specificities of the resulting polyclonal and/or monoclonal antibodies are checked by ELISA and/or western blotting using the immunogen, related constructs or whole cell lysates and extracts as targets. Negative controls, such as paralogous proteins, different constructs or different species are also employed to test specificity and/or to determine the range of species and/or genus cross-reactivity.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-26. (canceled)

27. Method of identifying an anti-fungal agent which targets ILV3 genes of fungi comprising contacting a candidate substance with
(i) a protein which comprises the sequence shown by SEQ ID NOs: 12, 21, 39, 42, 45, 48, 50, 53, 56, 59, 61 or 63, or
(ii) a protein which has at least 60% identity with (i), or
(iii) a protein comprising a fragment of (i) or (ii) which fragment has a length of at least 50 amino acids, and determining whether the candidate substance binds or modulates (i), (ii) or (iii), wherein binding or modulation of (i), (ii) or (iii) indicates that the candidate substance is an anti-fungal agent.

28. Method according to claim 27 comprising carrying out a reaction in the presence and absence of the candidate substance to determine whether the candidate substance inhibits the activity of the protein as defined in claim 27.

29. Method according to claim 27 comprising contacting a candidate substance with
(i) a protein which comprises the sequence shown by SEQ ID NOs: 21, 42, 45, 53 or 56, or
(ii) a protein which has at least 60% identity with (i), or
(iii) a protein comprising a fragment of (i) or (ii) which fragment has a length of at least 50 amino acids, and also contacting said candidate substance with
(iv) a protein which comprises the sequence shown by SEQ ID NOs: 12, 39, 48, 50 or 59, or
(v) a protein which has at least 60% identity with (i), or
(vi) a protein comprising a fragment of (i) or (ii) which fragment has a length of at least 50 amino acids, and determining whether the candidate substance binds or modulates (i), (ii) or (iii), and whether the candidate substance binds or modulates (iv), (v) or (vi), wherein binding or modulation of (i), (ii) or (iii), and (iv), (v) or (vi) indicates that the candidate substance is an anti-fungal agent.

30. Method according to claim 29 comprising carrying out a reaction in the presence and absence of the candidate substance to determine whether the candidate substance inhibits the activity of the proteins as defined in claim 29.

31. Method according to claim 27 comprising contacting a candidate substance with
(i) a protein which comprises the sequence shown by SEQ ID NOs: 12, 39, 48, 50 or 59, or
(ii) a protein which has at least 60% identity with (i), or
(iii) a protein comprising a fragment of (i) or (ii) which fragment has a length of at least 50 amino acids, and also contacting said candidate substance with
(iv) a protein which comprises the sequence shown by SEQ ID NOs: 21, 42, 45, 53 or 56, or
(v) a protein which has at least 60% identity with (iv), or
(vi) a protein comprising a fragment of (iv) or (v) which fragment has a length of at least 50 amino acids, and determining whether the candidate substance binds or modulates (i), (ii) or (iii), and whether the candidate substance binds or modulates (iv), (v) or (vi), wherein binding or modulation of (i), (ii) or (iii), and (iv), (v) or (vi) indicates that the candidate substance is an anti-fungal agent.

32. Method according to claim 31 comprising carrying out a reaction in the presence and absence of the candidate substance to determine whether the candidate substance inhibits the activity of the proteins as defined in claim 31.

33. Method according to claim 27 wherein the protein or polynucleotide is from Aspergillus flavus; Aspergillus fumigatus; Aspergillus nidulans; Aspergillus niger; Aspergillus parasiticus; Aspergillus terreus; Blumeria graminis; Candida albicans; Candida cruzei; Candida glabrata; Candida parapsilosis; Candida tropicalis; Colletotrichium trifolii; Cryptococcus neoformans; Encephalitozoon cuniculi; Fusarium graminarium; Fusarium solani; Fusarium sporotrichoides; Histoplasma capsulata; Leptosphaeria nodorum; Magnaporthe grisea; Mycosphaerella graminicola; Neurospora crassa; Phytophthora capsici; Phytophthora infestans; Plasmopara viticola; Pneumocystis jiroveci; Puccinia coronata; Puccinia graminis; Pyricularia oryzae; Pythium ultimum; Rhizoctonia solani; Saccharomyces cerevisiae; Schizosaccharomyces pombe; Trichophyton interdigitale; Trichophyton rubrum; or Ustilago maydis.

34. Method according to claim 27, which further comprises formulating the identified anti-fungal agent into an agricultural or a pharmaceutical composition.

35. Method according to claim 27, which further comprises killing or impairing the growth of a fungus by contacting the fungus with the identified anti-fungal agent.

36. A method of obtaining a protein as defined in claim 27, comprising expressing the protein from a polynucleotide as defined in claim 27, or a method of obtaining a polynucleotide as defined in claim 27 comprising replication of a vector or synthesis of the polynucleotide by condensation of nucleotides.

37. An organism which is transgenic for a polynucleotide as defined in claim 27, or an organism which has been genetically engineered to render a polynucleotide or protein as defined in claim 27 non-functional or inhibited or a fungus which has been killed, or whose growth has been impaired, by inhibition of the expression or activity of a protein or polynucleotide as defined in claim 27.

38. A method for preventing or treating a fungal infection comprising administering a protein or polynucleotide as defined in claim 27 or a method of killing, or impairing the growth of, a fungus comprising inhibiting the expression or activity of a polynucleotide or protein as defined in claim 27.

39. A method according to claim 38 wherein the fungus has infected a human, animal or plant individual.

40. A product which is:
an isolated protein or polynucleotide as defined in claim 27,
a vector comprising a polynucleotide as defined in claim 27,
a recombinant cell comprising a polynucleotide as defined in claim 27,
an antibody which is specific for a protein as defined in claim 27.


SEQUENCE LISTING (83 sequences)

SEQ ID NO: 1 - DNA, 1525 bp, Aspergillus fumigates
atgcgatcctttcctcttgtccttgccgcaggcattcctgccctggctgccaccgccggcgagttcaacatactcgccttgaacgtcgcaggactcccccccatcctgaacggcaacgacgtgcccggcgacaagtccgacaactctcggcagattggcaggaaattcgccgagtacggatacgacgtgattcatgtgcaggaggtgagtccgtccctgcgatcgccctgatctcgctgacgcaacaggacttcaactaccacgcgtacatctacgaaaccgacaaccatccctaccgcactcccacgtcgggcggcgcggggatcggttccggcttgaatacgctctccaactttgaattcaccaactttgtgcggaccaagtgggcgacctgctcgaatgccgagggggccgactgtctcacccccaaggggttcacctcgatgcgggtgcgcgttgaggagggggtctacgtggatttttacaatctccacgcggacgccgggtatttctttcctgccttcgtccgttctgtagcaaagctgacttgcacagatccaaagacgacgatgtcaaggctcgcagcgccaatctgcagcagctggccgattacatcaaggtcaactcggccggcaatgcggtgctggtgtttggcgacaccaatgcacggtatacaaggactggcgacaatatccgtgtcttccagacgcagaatggcatggtcaacccgtgggtggagctgattctccagggagccgcgccggccgaggggagcaatgccttgctctgccagaatcctagcacaaccagtgactgtgaaacggtcgataagatcttgtaaccgatccccgagccgtttgtgactgcacgacaggagggctgacgggaattagctaccggggaagccgtgcggtcgacctcagggccgtcttctggaactacgagagcaacaagttcctcagcgacaaggggacgatcctctccgatcacaatcccatcacgaccaacttcacctggaccttgtccaatgccttccgccagagtgacatcttcggtggcccacacgggtaaggcagattttttacatcagaaactcccaacgcggccagctaacacaagaagcagaacctggtttaacgacctcgactccctccctacggcgttcaccggccaaaacaagccaagcaagatctccctgcgcggtgccgagcgactcgacagcgtcggcctcaccgtggcctcgggcaagagctacacccacgggggcaccgggggccagctctcggaactccccctcgcggccaacgagtactggaccaaggcaaagctgtgccaggggcagtaccgcggccacacgcgcaacttctacctgctggcgacgacgagcagcggccggacggtgagtgcggggacggcgacgtcagactgcaaggagtttgcggcggcgccagggtggcagattgtgggattctacgggcaggacggggacgagattgatcagctgggatttctctacgggttgatt

SEQ ID NO: 2 - DNA, 1320 bp, Aspergillus fumigates
atgcgatcctttcctcttgtccttgccgcaggcattcctgccctggctgccaccgccggcgagttcaacatactcgccttgaacgtcgcaggactcccccccatcctgaacggcaacgacgtgcccggcgacaagtccgacaactctcggcagattggcaggaaattcgccgagtacggatacgacgtgattcatgtgcaggaggacttcaactaccacgcgtacatctacgaaaccgacaaccatccctaccgcactcccacgtcgggcggcgcggggatcggttccggcttgaatacgctctccaactttgaattcaccaactttgtgcggaccaagtgggcgacctgctcgaatgccgagggggccgactgtctcacccccaaggggttcacctcgatgcgggtgcgcgttgaggagggggtctacgtggatttttacaatctccacgcggacgccggatccaaagacgacgatgtcaaggctcgcagcgccaatctgcagcagctggccgattacatcaaggtcaactcggccggcaatgcggtgctggtgtttggcgacaccaatgcacggtatacaaggactggcgacaatatccgtgtcttccagacgcagaatggcatggtcaacccgtgggtggagctgattctccagggagccgcgccggccgaggggagcaatgccttgctctgccagaatcctagcacaaccagtgactgtgaaacggtcgataagatcttctaccggggaagccgtgcggtcgacctcagggccgtcttctggaactacgagagcaacaagttcctcagcgacaaggggacgatcctctccgatcacaatcccatcacgaccaacttcacctggaccttgtccaatgccttccgccagagtgacatcttcggtggcccacacggcagaacctggtttaacgacctcgactccctccctacggcgttcaccggccaaaacaagccaagcaagatctccctgcgcggtgccgagcgactcgacagcgtcggcctcaccgtggcctcgggcaagagctacacccacgggggcaccgggggccagctctcggaactccccctcgcggccaacgagtactggaccaaggcaaagctgtgccaggggcagtaccgcggccacacgcgcaacttctacctgctggcgacgacgagcagcggccggacggtgagtgcggggacggcgacgtcagactgcaaggagtttgcggcggcgccagggtggcagattgtgggattctacgggcaggacggggacgagattgatcagctgggatttctctacgggttgatt

SEQ ID NO: 3 - protein, 440 aa, Aspergillus fumigates
MRSFPLVLAAGIPALAATAGEFNILALNVAGLPPILNGNDVPGDKSDNSRQIGRKFAEYGYDVIHVQEDFNYHAYIYETDNHPYRTPTSGGAGIGSGLNTLSNFEFTNFVRTKWATCSNAEGADCLTPKGFTSMRVRVEEGVYVDFYNLHADAGSKDDDVKARSANLQQLADYIKVNSAGNAVLVFGDTNARYTRTGDNIRVFQTQNGMVNPWVELILQGAAPAEGSNALLCQNPSTTSDCETVDKIFYRGSRAVDLRAVFWNYESNKFLSDKGTILSDHNPITTNFTWTLSNAFRQSDIFGGPHGRTWFNDLDSLPTAFTGQNKPSKISLRGAERLDSVGLTVASGKSYTHGGTGGQLSELPLAANEYWTKAKLCQGQYRGHTRNFYLLATTSSGRTVSAGTATSDCKEFAAAPGWQIVGFYGQDGDEIDQLGFLYGLI

SEQ ID NO: 4 - DNA, 1599 bp, Aspergillus fumigates
atgaagggtcccacgggaggtcctcgtgaagacagcctcactggacttcttgtccgatcaacgtcgacgaactggtcgacgaattcggtgattgcggttgacgcgggtacccttgtttcagggattattcacactctggagcaatacaatgcggaattaagagatggctcgtatatgatgaatgaagggccttttgccggcctcagaatgccctacaagtctgctcaagccaatgctgcacatattttccgagacatcattggagctgtcctgatcacacatgctcacttggatcacctgtccggcctggcgatcaacacgcccatgcttgaagcaggaaacgggcccaaacctttggccgctctaccgtcaattgtcgctgccataaaaaaccatatgcttaacgacgtgatctggccaaatctgtccgatgaagatggcggggcgggtctactgacttatcaacgtcttgttgaaggggggaatccaaggttcgggcgtggagatgcgaaggggtatatccgagcctgcgacggtcttctagcccggtgcctcggcgtcagtcatggacgatgcaggcaacgctttcacccggagtctggtactcaccaccgcgtgggtagctcagtctttgctgctgatccactgatgctgccttccagagctatatccgttgatcactctacggacgcagggtaagtcttccacactggaacgcgattggaattcactgacattgaaactatttgaaggatatattctccagctcgctctccccgaatgcacccagcaaacacgaaagatcctatttgggcaacagtggagagctctgcattcttcatccgtgatcaccatactggcaatgagattattatcttcggtgacgtcgaacccgactgtgtctcgcttgacccacgcaataaacgcgtctgggaggcagcagcaccgaaaattgcgaccggtaaattgcgcgccatcttcattgaatgctcatatgacgattctgtcgaggatgcaactttatacggtcatctatgtccgcgacacctaattgcggaactgacgttgctggccgggaaggttatggaagcccggcacccgcgcatggcgatatcgagtatcggaaagcgtaaacactcagactctaacaggtatggcggcagtggtggccaagtgagcccgaaatccaagcgtttccaaagcttctccgttgcagctggcaagagggccgacatggattctgttacggccgacatgcgaccacgattctactcaaccgcggaaggcttggctgagatccctgagctggttcatgacgagccgacaccgagctaccacgagcctgacgatgtaagcgatactgaaaattcggtggatcctgcaccacagtccaccagccctggagaagctcagaccagtgactctgggcagcttccgctgtcgggtttatctatctacatcatacacatcaaagaagatctgaccgatgatatccctccccgagaacgaattttacaacagcttcgatcccgaggcgaagccgctagactaggctgcgagttctacgctccccatcgcggagaaggcatttgggtttga

SEQ ID NO: 5 - DNA, 1452 bp, Aspergillus fumigates
atgaagggtcccacgggaggtcctcgtgaagacagcctcactggacttcttgtccgatcaacgtcgacgaactggtcgacgaattcggtgattgcggttgacgcgggtacccttgtttcagggattattcacactctggagcaatacaatgcggaattaagagatggctcgtatatgatgaatgaagggccttttgccggcctcagaatgccctacaagtctgctcaagccaatgctgcacatattttccgagacatcattggagctgtcctgatcacacatgctcacttggatcacctgtccggcctggcgatcaacacgcccatgcttgaagcaggaaacgggcccaaacctttggccgctctaccgtcaattgtcgctgccataaaaaaccatatgcttaacgacgtgatctggccaaatctgtccgatgaagatggcggggcgggtctactgacttatcaacgtcttgttgaaggggggaatccaaggttcgggcgtggagatgcgaaggggtatatccgagcctgcgacggtcttctagcccggtgcctcggcgtcagtcatggacgatgcaggcaacgctttcacccggagtctggtactcaccaccgcgtggctcgctctccccgaatgcacccagcaaacacgaaagatcctatttgggcaacagtggagagctctgcattcttcatccgtgatcaccatactggcaatgagattattatcttcggtgacgtcgaacccgactgtgtctcgcttgacccacgcaataaacgcgtctgggaggcagcagcaccgaaaattgcgaccggtaaattgcgcgccatcttcattgaatgctcatatgacgattctgtcgaggatgcaactttatacggtcatctatgtccgcgacacctaattgcggaactgacgttgctggccgggaaggttatggaagcccggcacccgcgcatggcgatatcgagtatcggaaagcgtaaacactcagactctaacaggtatggcggcagtggtggccaagtgagcccgaaatccaagcgtttccaaagcttctccgttgcagctggcaagagggccgacatggattctgttacggccgacatgcgaccacgattctactcaaccgcggaaggcttggctgagatccctgagctggttcatgacgagccgacaccgagctaccacgagcctgacgatgtaagcgatactgaaaattcggtggatcctgcaccacagtccaccagccctggagaagctcagaccagtgactctgggcagcttccgctgtcgggtttatctatctacatcatacacatcaaagaagatctgaccgatgatatccctccccgagaacgaattttacaacagcttcgatcccgaggcgaagccgctagactaggctgcgagttctacgctccccatcgcggagaaggcatttgggtttga

SEQ ID NO: 6 - protein, 483 aa, Aspergillus fumigates
MKGPTGGPREDSLTGLLVRSTSTNWSTNSVIAVDAGTLVSGIIHTLEQYNAELRDGSYMMNEGPFAGLRMPYKSAQANAAHIFRDIIGAVLITHAHLDHLSGLAINTPMLEAGNGPKPLAALPSIVAAIKNHMLNDVIWPNLSDEDGGAGLLTYQRLVEGGNPRFGRGDAKGYIRACDGLLARCLGVSHGRCRQRFHPESGTHHRVARSPRMHPANTKDPIWATVESSAFFIRDHHTGNEIIIFGDVEPDCVSLDPRNKRVWEAAAPKIATGKLRAIFIECSYDDSVEDATLYGHLCPRHLIAELTLLAGKVMEARHPRMAISSIGKRKHSDSNRYGGSGGQVSPKSKRFQSFSVAAGKRADMDSVTADMRPRFYSTAEGLAEIPELVHDEPTPSYHEPDDVSDTENSVDPAPQSTSPGEAQTSDSGQLPLSGLSIYIIHIKEDLTDDIPPRERILQQLRSRGEAARLGCEFYAPHRGEGIWV

SEQ ID NO: 7 - DNA, 1434 bp, Aspergillus fumigates
atggtggccgaacacttgacgattcgcaaccttaccacaaccccaataatcctcaagctcattgagcgttttcacccccataaggatcctcgagatgacatacattccttggccaggaattttactcgaattctaagcaatgtcactcgcaccaatgagactgtcgccgcgattactgatgacaatgaaccatttgctcatgaagagtatgatattcacgtggagcctttccagacagtccagaccgaagttcgtgcattcatcgattcggacaaagagcgcctgcgctggacctttgaagccgagggagagcgacatcagattcagacaccggttcctacgacggagtctgccggaatgaaggcgttgtgtgataatcctcgatttcgcttcactggcgtctacgtgacgcctgaatcccaccttgcgatctactcctcggccaatctgcaagcatggatgggagagctcaaagatagcacattgctctcctcgctatccataccaggaacacacaattctcctacgtgccatgtcgcggccccgtctgtccgctgccaagctgtcagtccgcgcgaacagcttcgaaatggtgtccgtttcttcgacatccgtgtgcagccccagttcccagaggacccatccaaagatgagcttatcctagtgcacagcgtgttccccatctccttgacgggcaacaagtactttcgcgatctgatgcgagacgttaatgagttcctcaatgagaatccatccgagacgctcatcatctctctgaagcgagaaggcccaggaaaccataccgatgaacaattgagtcggattgtgcgcgaccattatgctcgtccagatagccggtggtacacagaacccaaaatccctactctgggcgaggtgcgcgggaaagtcgtcctgcttcgccggttcaacatcattgaagagctaaagcatgaacacgacggccgcggctggggcatagacggaagcgattgggcagataatactccaaacgctacctgcagcagtggtcaactctgcatccaggacttctacgaggtcctcgagacgaagaacatcgatgtcaaaatcaaatatgtaacggagcactgtgagcgctccagcgggcactgttacccgttcggtgccctcccggatcccgaagctagcaaagcgcatccattctatataaatttcctcagtgcaagcaacttctggaaggtggggacgtggcccgagaagattgcagcaaagctgaacccggcaactgttgactatctctgccggagacacagtcacccggacggtgactggtcgactggtattcttgttacggactgggttggccacgaaggcgactgggaccttgtgcgttgcattgttggcatgaatgctaagctgaagatgagacagatgagagaggagcaggaacactaa

SEQ ID NO: 8 - DNA, 1434 bp, Aspergillus fumigates; identical to SEQ ID NO: 7

SEQ ID NO: 9 - protein, 477 aa, Aspergillus fumigates
MVAEHLTIRNLTTTPIILKLIERFHPHKDPRDDIHSLARNFTRILSNVTRTNETVAAITDDNEPFAHEEYDIHVEPFQTVQTEVRAFIDSDKERLRWTFEAEGERHQIQTPVPTTESAGMKALCDNPRFRFTGVYVTPESHLAIYSSANLQAWMGELKDSTLLSSLSIPGTHNSPTCHVAAPSVRCQAVSPREQLRNGVRFFDIRVQPQFPEDPSKDELILVHSVFPISLTGNKYFRDLMRDVNEFLNENPSETLIISLKREGPGNHTDEQLSRIVRDHYARPDSRWYTEPKIPTLGEVRGKVVLLRRFNIIEELKHEHDGRGWGIDGSDWADNTPNATCSSGQLCIQDFYEVLETKNIDVKIKYVTEHCERSSGHCYPFGALPDPEASKAHPFYINFLSASNFWKVGTWPEKIAAKLNPATVDYLCRRHSHPDGDWSTGILVTDWVGHEGDWDLVRCIVGMNAKLKMRQMREEQEH

SEQ ID NO: 10 - DNA, 1965 bp, Aspergillus fumigates
atggactcctctacctccgcgtcgtccaacacccacggcgaagccaagtatatcaacttccccactcttcctgacgacgcaaagcatgaagatggcaccactgcgctgaacagatattcttcttatatcactcggggccatgactttcctggtgctcgggtttgttaccctcattgagctgtctatgagatgtgggggctgacaatctgctcttgtatgactgaataggctatgctttttgcagcggggatcccggatcgcgaagcgatggctaagagcccacaggtaggaattgccagtgtctggtgggagggaaatccttgtaatatgcatctgctggacttgggcaagaccgtgaagaaggccgttacagatcagggtatgatcggttggcagtataataccattggagtttcagatgccatttcaatgggtagtgagggtgagcgtattcagggcttgaggagcattgctactctctgcgtgagatattcctgacgtgtattcccaggcatgagattttctctccagacgcgtgagatcattgcagacagcgtcgagactgtgacttgcgcgcagtatcatgatgcatgcattgcaattcctgggtgcgacaagaatatgcctggagtagtaatgggtatggccagacacaatcggccttcgcttatgatttacggtggaacaattcaggttggatactcgaacctgctgcggaagcgggtcaacgtgtcgacttgctttgaagcggctggtgcctatgcttatgatactttgcgtcaaccggacgatgggggtgacaccagtaaaagcaaggacgagattatggatgacattgagagacatgcttgtcccagtgcgggtgcatgtggaggcatgtttactgcaaacacaatggccacggcgattgagtctatgggcctgtccctaccagggtcatcgtcaacgcctgcctcgtctccatcgaagatgcgagaatgtgttaaagcggcagaagccatcaagacctgtatggagaagaacattaggcctcgggatcttttgaccaagcgctccttcgagaatgccctcgtcatgacgatggctctgggaggaagtaccaatggtgtcttgcatttccttgccatggctcggacggcggatgtgaacctgaccctagatgatgtccaacgggtcagcaacaagatccctttcattgctgacttggcccccagtgggaagtactacatggcagacctgtacgatatcggagggatcccgtccgtgcagaagttgctgatcgcggcgggacttcttgacggtgacatcccgacggtcaccggcaagaccttggctgagaatgttgcatctttcccatctctacctcaggaccaagtcatcatccggcccctggacaacccaatcaagacgactggccacctgcagattctacgcgggaacctggcgcctggcggagcggtggccaagatcactggcaaggagggcaccaagttcacaggcaaagcacgtgttttcgataaagaatatcagctcaacgatgctctgacccaaggcaagattcctcgaggcgaaaacttagtgctcatcgtccgctacgaaggacccaagggtgggccaggcatgccggagcagctcaaagcgagcgcggcgctgatgggagctaagctcaacaatgtggccctaatcacagatggaagatattcaggggctagtcatggattcatcgtgggtcatatcgtcccagaagctgcggtcggagggcccattgccattgttcgcgatgacgatgtgatcaccattgatgcggaaaccaacacgataaacatgcatgtctcagatgaggaaatccagcagcgactgaaagagtggaagcccccagtgcctcatgtcacacgtggtgtactcgccaagtatgcaaggctggttggggatgcctctcatggtgcaatgacggatttgttctag

SEQ ID NO: 11 - DNA, 1827 bp, Aspergillus fumigates
atggactcctctacctccgcgtcgtccaacacccacggcgaagccaagtatatcaacttccccactcttcctgacgacgcaaagcatgaagatggcaccactgcgctgaacagatattcttcttatatcactcggggccatgactttcctggtgctcgggctatgctttttgcagcggggatcccggatcgcgaagcgatggctaagagcccacaggtaggaattgccagtgtctggtgggagggaaatccttgtaatatgcatctgctggacttgggcaagaccgtgaagaaggccgttacagatcagggtatgatcggttggcagtataataccattggagtttcagatgccatttcaatgggtagtgagggcatgagattttctctccagacgcgtgagatcattgcagacagcgtcgagactgtgacttgcgcgcagtatcatgatgcatgcattgcaattcctgggtgcgacaagaatatgcctggagtagtaatgggtatggccagacacaatcggccttcgcttatgatttacggtggaacaattcaggttggatactcgaacctgctgcggaagcgggtcaacgtgtcgacttgctttgaagcggctggtgcctatgcttatgatactttgcgtcaaccggacgatgggggtgacaccagtaaaagcaaggacgagattatggatgacattgagagacatgcttgtcccagtgcgggtgcatgtggaggcatgtttactgcaaacacaatggccacggcgattgagtctatgggcctgtccctaccagggtcatcgtcaacgcctgcctcgtctccatcgaagatgcgagaatgtgttaaagcggcagaagccatcaagacctgtatggagaagaacattaggcctcgggatcttttgaccaagcgctccttcgagaatgccctcgtcatgacgatggctctgggaggaagtaccaatggtgtcttgcatttccttgccatggctcggacggcggatgtgaacctgaccctagatgatgtccaacgggtcagcaacaagatccctttcattgctgacttggcccccagtgggaagtactacatggcagacctgtacgatatcggagggatcccgtccgtgcagaagttgctgatcgcggcgggacttcttgacggtgacatcccgacggtcaccggcaagaccttggctgagaatgttgcatctttcccatctctacctcaggaccaagtcatcatccggcccctggacaacccaatcaagacgactggccacctgcagattctacgcgggaacctggcgcctggcggagcggtggccaagatcactggcaaggagggcaccaagttcacaggcaaagcacgtgttttcgataaagaatatcagctcaacgatgctctgacccaaggcaagattcctcgaggcgaaaacttagtgctcatcgtccgctacgaaggacccaagggtgggccaggcatgccggagcagctcaaagcgagcgcggcgctgatgggagctaagctcaacaatgtggccctaatcacagatggaagatattcaggggctagtcatggattcatcgtgggtcatatcgtcccagaagctgcggtcggagggcccattgccattgttcgcgatgacgatgtgatcaccattgatgcggaaaccaacacgataaacatgcatgtctcagatgaggaaatccagcagcgactgaaagagtggaagcccccagtgcctcatgtcacacgtggtgtactcgccaagtatgcaaggctggttggggatgcctctcatggtgcaatgacggatttgttctag

SEQ ID NO: 12 - protein, 608 aa, Aspergillus fumigates
MDSSTSASSNTHGEAKYINFPTLPDDAKHEDGTTALNRYSSYITRGHDFPGARAMLFAAGIPDREAMAKSPQVGIASVWWEGNPCNMHLLDLGKTVKKAVTDQGMIGWQYNTIGVSDAISMGSEGMRFSLQTREIIADSVETVTCAQYHDACIAIPGCDKNMPGVVMGMARHNRPSLMIYGGTIQVGYSNLLRKRVNVSTCFEAAGAYAYDTLRQPDDGGDTSKSKDEIMDDIERHACPSAGACGGMFTANTMATAIESMGLSLPGSSSTPASSPSKMRECVKAAEAIKTCMEKNIRPRDLLTKRSFENALVMTMALGGSTNGVLHFLAMARTADVNLTLDDVQRVSNKIPFIADLAPSGKYYMADLYDIGGIPSVQKLLIAAGLLDGDIPTVTGKTLAENVASFPSLPQDQVIIRPLDNPIKTTGHLQILRGNLAPGGAVAKITGKEGTKFTGKARVFDKEYQLNDALTQGKIPRGENLVLIVRYEGPKGGPGMPEQLKASAALMGAKLNNVALITDGRYSGASHGFIVGHIVPEAAVGGPIAIVRDDDVITIDAETNTINMHVSDEEIQQRLKEWKPPVPHVTRGVLAKYARLVGDASHGAMTDLF

SEQ ID NO: 13 - DNA, 831 bp, Aspergillus fumigates
atggctaaaccacttagtgagaagtcttctaatagctccctcagggctaaagcgagtgagtcttcttcccttggttatcgacctttgcagtgagctgtttactgattagaccgcgttaaccaggcgaaaggactcgtcttagagaaacatcattggagtcagagatcattcacactgaaaagttgcctcccaatttcggagatgttgtcaaaggagtctatagaagctctttcccgcaaccttggcatttccaagcgctaaagaagctgggactcaggatgattgtgtaagcggctgccatataagatcattacccctttcttacgcttgtccacatagtacgttagttgaaggggactacacccaggaccaccaagtctttctcaaagagaatggtattgaacatcgtcgcattcttatcctggcaaacaaggatcccacgattcgaactccggaccatgtcgtgaaccgagtcttggaaatcatgctcaacaagaccaatcacccactccttctacactgcaacaagggaaaggtgagcgcaaatgccgataatacaaattcgacccgactaacaagacttaatagcatcggactggatgcattgtcggctgctttcggaaggttcagggctgggacatgccagctattcgcaaggaatacctcaatttttcgttgccgaaatcaaggcctctcgacgaacgattcattgaacttttcgatgacaccagactcgggcccctcgctgtttcttctggtgcaagctcctggcctgctggtgtaatgctcgatccgcttcgcgaagaagtagtcgaggacgaaaataccccg

SEQ ID NO: 14 - DNA, 672 bp, Aspergillus fumigates
atggctaaaccacttagtgagaagtcttctaatagctccctcagggctaaagcgagtgagtcttcttcccttggttatcgacctttgcaagaaacatcattggagtcagagatcattcacactgaaaagttgcctcccaatttcggagatgttgtcaaaggagtctatagaagctctttcccgcaaccttggcatttccaagcgctaaagaagctgggactcaggatgattgttacgttagttgaaggggactacacccaggaccaccaagtctttctcaaagagaatggtattgaacatcgtcgcattcttatcctggcaaacaaggatcccacgattcgaactccggaccatgtcgtgaaccgagtcttggaaatcatgctcaacaagaccaatcacccactccttctacactgcaacaagggaaagcatcggactggatgcattgtcggctgctttcggaaggttcagggctgggacatgccagctattcgcaaggaatacctcaatttttcgttgccgaaatcaaggcctctcgacgaacgattcattgaacttttcgatgacaccagactcgggcccctcgctgtttcttctggtgcaagctcctggcctgctggtgtaatgctcgatccgcttcgcgaagaagtagtcgaggacgaaaataccccg

SEQ ID NO: 15 - protein, 224 aa, Aspergillus fumigates
MAKPLSEKSSNSSLRAKASESSSLGYRPLQETSLESEIIHTEKLPPNFGDVVKGVYRSSFPQPWHFQALKKLGLRMIVTLVEGDYTQDHQVFLKENGIEHRRILILANKDPTIRTPDHVVNRVLEIMLNKTNHPLLLHCNKGKHRTGCIVGCFRKVQGWDMPAIRKEYLNFSLPKSRPLDERFIELFDDTRLGPLAVSSGASSWPAGVMLDPLREEVVEDENTP

SEQ ID NO: 16 - DNA, 1011 bp, Aspergillus fumigates
atggacacccccgatctcaaatgcacccatctcacctcaccagactatatgaacttcgtcctgtctatgtacaaccctgacgttgccgcaatagctctcgtgaacttcaactaacgaacacagtcttatcatatttgggattctcctgtcgtatctccctcagcacattcgcataatcaacctcaaaagctccttcgggatctccccatacttcgtcctccttggaaccacatcaggtacctcggcgttggcaaatgttgtaacacaacagcagagtttacatgatgtggaatgctgcaagaacatcaatggactagcttgctttgggggactactcggcatcttccaagtcgggacacagtggctttgctttgttatcatgtaagctcacctacttctccgcctgataacattcacgtgattaatgcaagatcgtgtagtctgcttttgttcgttatttacttcccccgagccacctctccaatctcgcctacagaatcagagtcttcggcgagaaatggcccatcatatacaacagcgttggtcgtcagcggtatttgtatcctccacgccgtggtgatgttcatcacctctgccgcaatcgcggtcaaccggccatcccagcttcaggcatggtccaacttttcaggggtggttgctgccattctcgcttcgatccagtatttccctcagatctacactacgttaaggttgcggtgtgtgggtagcctgagcatcccaatgatgtgtatccaaaccccgggaagtcttgtgtgggcaggtagtttagctgcacgactgggaccaaaaggatggagtacatggggcgtgttgattgtgacggcatgcttacaaggcacgctcttggcaatggccatcttttttgaatattttgggcccaacaagcagcgcaaccatcgccatggcaaagatcttcctccgaacggtagtggagaaggcccagaggaaagagaccatgagcagccgtctgaggaaacgccacttctccaa

SEQ ID NO: 17 - DNA, 897 bp, Aspergillus fumigates
atggacacccccgatctcaaatgcacccatctcacctcaccagactatatgaacttcgtcctgtctattcttatcatatttgggattctcctgtcgtatctccctcagcacattcgcataatcaacctcaaaagctccttcgggatctccccatacttcgtcctccttggaaccacatcaggtacctcggcgttggcaaatgttgtaacacaacagcagagtttacatgatgtggaatgctgcaagaacatcaatggactagcttgctttgggggactactcggcatcttccaagtcgggacacagtggctttgctttgttatcattctgcttttgttcgttatttacttcccccgagccacctctccaatctcgcctacagaatcagagtcttcggcgagaaatggcccatcatatacaacagcgttggtcgtcagcggtatttgtatcctccacgccgtggtgatgttcatcacctctgccgcaatcgcggtcaaccggccatcccagcttcaggcatggtccaacttttcaggggtggttgctgccattctcgcttcgatccagtatttccctcagatctacactacgttaaggttgcggtgtgtgggtagcctgagcatcccaatgatgtgtatccaaaccccgggaagtcttgtgtgggcaggtagtttagctgcacgactgggaccaaaaggatggagtacatggggcgtgttgattgtgacggcatgcttacaaggcacgctcttggcaatggccatcttttttgaatattttgggcccaacaagcagcgcaaccatcgccatggcaaagatcttcctccgaacggtagtggagaaggcccagaggaaagagaccatgagcagccgtctgaggaaacgccacttctccaa

SEQ ID NO: 18 - protein, 299 aa, Aspergillus fumigates
MDTPDLKCTHLTSPDYMNFVLSILIIFGILLSYLPQHIRIINLKSSFGISPYFVLLGTTSGTSALANVVTQQQSLHDVECCKNINGLACFGGLLGIFQVGTQWLCFVIILLLFVIYFPRATSPISPTESESSARNGPSYTTALVVSGICILHAVVMFITSAAIAVNRPSQLQAWSNFSGVVAAILASIQYFPQIYTTLRLRCVGSLSIPMMCIQTPGSLVWAGSLAARLGPKGWSTWGVLIVTACLQGTLLAMAIFFEYFGPNKQRNHRHGKDLPPNGSGEGPEERDHEQPSEETPLLQ

SEQ ID NO: 19 - DNA, 1963 bp, Aspergillus fumigates
atgcttctctctcagaccagagggcgtctgccctcgactctccggagcttctctcggtaggccacgctgctctccaaataggtcctttgaactaagaaggatcgccgttaacgatatatttcaatccagccgtgccctttcgactactctccccagaggcaaagatagcgaagaaacggctttgaataaggtctcccgcaatgtcacacaacccatctcgcagggtgcttcccaggctatgctgtacgctacaggcctcacagaggaggatatgaacaaggcgcaggtcggtatttcgtctgtctggtacaacggcaacccctgtaacatgcacctgctggatctcagcaaccgggtgcgtgaaggtgtgcaaaaggccggtctggtcggcttccagttcaacaccgttggtgtcagcgacgccatcagtatgggtaccaagggaatgcgatactctctgcagagccgtgatctgatcgccgattccatcgaaaccgtcatgggtggccagtggtacgacgcgaacatcagtatccccggttgcgacaagaacatgcccggtgtgttgatggccatgggtcgtgtcaaccgccccagtctgatggtgtacggcggtaccatcaagcccggctgcgccaggactcagaacaacgcagacatcgatatcgtttcggccttccaggcgtacggacagttcctgaccggcgagatcaccgagaaccagcgctttgacatcatccgcaacgcctgccccggcggtggtgcctgtggcggtatgtacacggccaacaccatggcgaccgccatcgaggtcatgggtatgacgctgcccggctcctcgtcgaacccggccgagtccaaggccaaggacctcgaatgcttggcggccggtgaggccatcaagcggctgctcaaggaggacattcggccgtcggacatcctgactcgccaggccttcgagaacgccatgatcgtcgtcaacatcaccggtggctcgaccaatgccgtcctccacctgatcgccatcgccgactcggttggcatcaagctcgacatcgaggacttccagaaggtctcggaccgcacccctttcctggccgacctgaagccatcgggcaagtacgtcatggctgacctgcacaacatcggcggcaccccctccctgctcaagttcctgctcaaggagggcgtcatcgacggctccggcatgaccgtcacgggtgagacccttgccaagaacctcgagaaggttcccgacttcccagccgaccagaagatcatccggcccctgtccaaccccatcaagaagaccggccacattcagatcctccgcggctccctggcccccggcggctccgtcggcaagatcaccggcaaggaaggcacccgcttcgtcggcaaggcccgcgtcttcgacgacgaggacgatttcatcgccgctctcgagcgcaacgagatcaaaaaggaggaaaagaccgtcgttgtcatccgctacaccggccccaagggcggacccggcatgcccgagatgctcaagccctcctccgcactcatgggcgccggcctcggctcctcctgcgctctcatcaccgacggccgcttctccggcggctctcacggcttcctgatcggtcacatcgtgcccgaggccgcggtcggtggtcccatcggtctcgtcaaggatggcgacaccatcaccatcgacgccgagaagcgcctgctcgacctcgacgttgacgagaccgagcttgctcgtcgaaggaaggagtgggaggctctccgggatgccggcaagttgcctcaaactggtcttacgatgaggggtaccctgggtaaatacgctaggtatgttccgcctcacctattttcccctggtctttggtttcctaacgatagcatactgactggctgtagaactgtcaaggatgccagccacggctgcatcactgactctgtagaatga

SEQ ID NO: 20 - DNA, 1821 bp, Aspergillus fumigates
atgcttctctctcagaccagagggcgtctgccctcgactctccggagcttctctcgccgtgccctttcgactactctccccagaggcaaagatagcgaagaaacggctttgaataaggtctcccgcaatgtcacacaacccatctcgcagggtgcttcccaggctatgctgtacgctacaggcctcacagaggaggatatgaacaaggcgcaggtcggtatttcgtctgtctggtacaacggcaacccctgtaacatgcacctgctggatctcagcaaccgggtgcgtgaaggtgtgcaaaaggccggtctggtcggcttccagttcaacaccgttggtgtcagcgacgccatcagtatgggtaccaagggaatgcgatactctctgcagagccgtgatctgatcgccgattccatcgaaaccgtcatgggtggccagtggtacgacgcgaacatcagtatccccggttgcgacaagaacatgcccggtgtgttgatggccatgggtcgtgtcaaccgccccagtctgatggtgtacggcggtaccatcaagcccggctgcgccaggactcagaacaacgcagacatcgatatcgtttcggccttccaggcgtacggacagttcctgaccggcgagatcaccgagaaccagcgctttgacatcatccgcaacgcctgccccggcggtggtgcctgtggcggtatgtacacggccaacaccatggcgaccgccatcgaggtcatgggtatgacgctgcccggctcctcgtcgaacccggccgagtccaaggccaaggacctcgaatgcttggcggccggtgaggccatcaagcggctgctcaaggaggacattcggccgtcggacatcctgactcgccaggccttcgagaacgccatgatcgtcgtcaacatcaccggtggctcgaccaatgccgtcctccacctgatcgccatcgccgactcggttggcatcaagctcgacatcgaggacttccagaaggtctcggaccgcacccctttcctggccgacctgaagccatcgggcaagtacgtcatggctgacctgcacaacatcggcggcaccccctccctgctcaagttcctgctcaaggagggcgtcatcgacggctccggcatgaccgtcacgggtgagacccttgccaagaacctcgagaaggttcccgacttcccagccgaccagaagatcatccggcccctgtccaaccccatcaagaagaccggccacattcagatcctccgcggctccctggcccccggcggctccgtcggcaagatcaccggcaaggaaggcacccgcttcgtcggcaaggcccgcgtcttcgacgacgaggacgatttcatcgccgctctcgagcgcaacgagatcaaaaaggaggaaaagaccgtcgttgtcatccgctacaccggccccaagggcggacccggcatgcccgagatgctcaagccctcctccgcactcatgggcgccggcctcggctcctcctgcgctctcatcaccgacggccgcttctccggcggctctcacggcttcctgatcggtcacatcgtgcccgaggccgcggtcggtggtcccatcggtctcgtcaaggatggcgacaccatcaccatcgacgccgagaagcgcctgctcgacctcgacgttgacgagaccgagcttgctcgtcgaaggaaggagtgggaggctctccgggatgccggcaagttgcctcaaactggtcttacgatgaggggtaccctgggtaaatacgctagaactgtcaaggatgccagccacggctgcatcactgactctgtagaatga

SEQ ID NO: 21 - protein, 606 aa, Aspergillus fumigates
MLLSQTRGRLPSTLRSFSRRALSTTLPRGKDSEETALNKVSRNVTQPISQGASQAMLYATGLTEEDMNKAQVGISSVWYNGNPCNMHLLDLSNRVREGVQKAGLVGFQFNTVGVSDAISMGTKGMRYSLQSRDLIADSIETVMGGQWYDANISIPGCDKNMPGVLMAMGRVNRPSLMVYGGTIKPGCARTQNNADIDIVSAFQAYGQFLTGEITENQRFDIIRNACPGGGACGGMYTANTMATAIEVMGMTLPGSSSNPAESKAKDLECLAAGEAIKRLLKEDIRPSDILTRQAFENAMIVVNITGGSTNAVLHLIAIADSVGIKLDIEDFQKVSDRTPFLADLKPSGKYVMADLHNIGGTPSLLKFLLKEGVIDGSGMTVTGETLAKNLEKVPDFPADQKIIRPLSNPIKKTGHIQILRGSLAPGGSVGKITGKEGTRFVGKARVFDDEDDFIAALERNEIKKEEKTVVVIRYTGPKGGPGMPEMLKPSSALMGAGLGSSCALITDGRFSGGSHGFLIGHIVPEAAVGGPIGLVKDGDTITIDAEKRLLDLDVDETELARRRKEWEALRDAGKLPQTGLTMRGTLGKYARTVKDASHGCITDSVE

SEQ ID NO: 22 - DNA, 1704 bp, Aspergillus fumigates
atgacgaatcgtacatcattgaacggacggtgtccggtaccattccttcaggaagatctcttcccccccactggtggatgtacgctcccctgctgattgctgccgtgctgttgtcgacattcgctgatcgtgttggttctttgcttgctagtcatcggaggtcgttattgtcaacctgtaggcgatatttcgtgctgtttaccctgtccgatagtatcatggacgtacggtgatggtaagttcacagtgtgttcgagttgcagagtcacccatcattctaacctcgttcactttttcaagggttgttcgggaaggcgagctctgccagctggatcagtgttgctatcttgccgctctgtatcttcctgcttgtgtcgtacgcagtgctcccggtcaaatttacgcatcgccactacctcagcgtctgcttcacattgggtatctgtttcatggaggcaagtaaagtggcgcgttggagggacctacgacctgttattaaccgacatgcagattgcgttcattatccctctaggggtaaaaccagatcaatgctataaccagatcacccccaacgatatgcattctagtctctcctgcgcttttacaggctcccttcttcttctcggaggctggatggtggtagtatggagtatgttgggaacaattctgagcttatttggattgccctcgttctaacgatctccaggttttctccgtacggtggccttccatcttcaggtatgctgggaagtcatactgggtccgaaattcatgtggggagcgttgatcttcggttgggtcgtcccggctgtcggcctcacggtgatgctgatcttgacgggcgtatcttttcgattcggcaccgtttgtcatatcaacatcgatggtgccctgcaggattattggattcccatcatctcgttcgcggttgccgcactgatcctccaactggcgacaatggcgtactgcatccacgtctacgtcaagtctttgttcgacaccgattcgacgacaaacagctcgggattgccgtcctactctgccagcgtccgcaccgtgtccgctcgtcaagcataccgtcgcatccgcagagtcctccaattacagtggcgaggcgtaactctggtcttgatcatcatcgcgaatgtgattttcttctccgtgaccttcatcgagctcgacagctccctcaaaccgaccgcggagaatatggaaaaggcgcttccatgggttgcatgtctggcagccaccaatggtgaccgagaaaaatgcgaccccgaggcggcaaagttccggcctagcgaagggttactcctcgccgtcctggttctcctttccctggtcggattctggaacttcatcctctttgcccgcccctcgatcttccacggctgggtcgacttcttccagaacaagtttggcacgggcgacggccgtctcgagttcgtgtcggctgatgcccgcacgcggctgggcgacacgcggtcctacgagatgctcaacagcacgggcctcccctcctacaaatcgccatcgcccatggtccgatcgccaagtcctgcgcgcatggggggcacgaagagtcccgagaatggccactttgggcgagacgccaggtatgtgcggccgtccatgagtttctccagcccgcggccgcccagcgcatcacaagggcgcggctgggatcccaagacaacgtttgcc

SEQ ID NO: 23 - DNA, 1464 bp, Aspergillus fumigates
atgacgaatcgtacatcattgaacggacggtgtccggtaccattccttcaggaagatctcttcccccccactggtggattcatcggaggtcgttattgtcaacctgtaggcgatatttcgtgctgtttaccctgtccgatagtatcatggacgtacggtgatgggttgttcgggaaggcgagctctgccagctggatcagtgttgctatcttgccgctctgtatcttcctgcttgtgtcgtacgcagtgctcccggtcaaatttacgcatcgccactacctcagcgtctgcttcacattgggtatctgtttcatggaggcaagtaaaattgcgttcattatccctctaggggtaaaaccagatcaatgctataaccagatcacccccaacgatatgcattctagtctctcctgcgcttttacaggctcccttcttcttctcggaggctggatggtggtagtatggagttttctccgtacggtggccttccatcttcaggtatgctgggaagtcatactgggtccgaaattcatgtggggagcgttgatcttcggttgggtcgtcccggctgtcggcctcacggtgatgctgatcttgacgggcgtatcttttcgattcggcaccgtttgtcatatcaacatcgatggtgccctgcaggattattggattcccatcatctcgttcgcggttgccgcactgatcctccaactggcgacaatggcgtactgcatccacgtctacgtcaagtctttgttcgacaccgattcgacgacaaacagctcgggattgccgtcctactctgccagcgtccgcaccgtgtccgctcgtcaagcataccgtcgcatccgcagagtcctccaattacagtggcgaggcgtaactctggtcttgatcatcatcgcgaatgtgattttcttctccgtgaccttcatcgagctcgacagctccctcaaaccgaccgcggagaatatggaaaaggcgcttccatgggttgcatgtctggcagccaccaatggtgaccgagaaaaatgcgaccccgaggcggcaaagttccggcctagcgaagggttactcctcgccgtcctggttctcctttccctggtcggattctggaacttcatcctctttgcccgcccctcgatcttccacggctgggtcgacttcttccagaacaagtttggcacgggcgacggccgtctcgagttcgtgtcggctgatgcccgcacgcggctgggcgacacgcggtcctacgagatgctcaacagcacgggcctcccctcctacaaatcgccatcgcccatggtccgatcgccaagtcctgcgcgcatggggggcacgaagagtcccgagaatggccactttgggcgagacgccaggtatgtgcggccgtccatgagtttctccagcccgcggccgcccagcgcatcacaagggcgcggctgggatcccaagacaacgtttgcc

SEQ ID NO: 24 - protein, 488 aa, Aspergillus fumigates
MTNRTSLNGRCPVPFLQEDLFPPTGGFIGGRYCQPVGDISCCLPCPIVSWTYGDGLFGKASSASWISVAILPLCIFLLVSYAVLPVKFTHRHYLSVCFTLGICFMEASKIAFIIPLGVKPDQCYNQITPNDMHSSLSCAFTGSLLLLGGWMVVVWSFLRTVAFHLQVCWEVILGPKFMWGALIFGWVVPAVGLTVMLILTGVSFRFGTVCHINIDGALQDYWIPIISFAVAALILQLATMAYCIHVYVKSLFDTDSTTNSSGLPSYSASVRTVSARQAYRRIRRVLQLQWRGVTLVLIIIANVIFFSVTFIELDSSLKPTAENMEKALPWVACLAATNGDREKCDPEAAKFRPSEGLLLAVLVLLSLVGFWNFILFARPSIFHGWVDFFQNKFGTGDGRLEFVSADARTRLGDTRSYEMLNSTGLPSYKSPSPMVRSPSPARMGGTKSPENGHFGRDARYVRPSMSFSSPRPPSASQGRGWDPKTTFA

SEQ ID NO: 25 - DNA, 957 bp, Aspergillus fumigates
atgaagacaactaccgaacttccactgcgtatactaacacacaatatccgctatgcaaccagttccccattcaagggtgagctgccctggaacgatcgcaagcagcctctcttaaatgaactgctcttcaatacgcgcaatcaggatgcattcatctgcttacaagaagtgctccataatcaactggtcgatgttctatccggactgaaacaacctccttctactattcccaaaagcgcctccgagacacaacaatgggaatacatcggagtcggtcgagacgacgggcacaaagcaggagagtactcgcccatcttctatcaaccgtctgtttggcagctgtgtcattgggaaagtgtctggttgtcggaaacgccgaacaaaccatccaaaggctgggatgcggcgtctataaggattctggtaagacactaggcgtgtgacgaaatcctcttcattaagttgacgccaaactagaccatcggtgtcttcacacacaacataactcgccataccgtccttgtgatgaatacacatctagacgaccagggatctcagtcacgcttcgaggctgccaaaatcatccttcagaagatcgatgaataccgaagcggcaaatttgggacacttatcgcaggggtattcctcgcaggtgattttaatagtcaagagacgcaagaagcctataatgtcttgacagggtcggaatcctccttggtcgacacagcaaaggttgtggaacctagccagcactacggcaattattatacatggacagggttcggatatgagggagaggatcccacgcgcattgattatattttaatcggacctgggaagaacaaacttgggtcctggatagtcaacggatacgccgtgttagcgaaccgattcgattctggcgtgtttctgtcagaccacagggccgttgtcgcagacatcaccttgtacaactga

SEQ ID NO: 26 - DNA, 903 bp, Aspergillus fumigates
atgaagacaactaccgaacttccactgcgtatactaacacacaatatccgctatgcaaccagttccccattcaagggtgagctgccctggaacgatcgcaagcagcctctcttaaatgaactgctcttcaatacgcgcaatcaggatgcattcatctgcttacaagaagtgctccataatcaactggtcgatgttctatccggactgaaacaacctccttctactattcccaaaagcgcctccgagacacaacaatgggaatacatcggagtcggtcgagacgacgggcacaaagcaggagagtactcgcccatcttctatcaaccgtctgtttggcagctgtgtcattgggaaagtgtctggttgtcggaaacgccgaacaaaccatccaaaggctgggatgcggcgtctataaggattctgaccatcggtgtcttcacacacaacataactcgccataccgtccttgtgatgaatacacatctagacgaccagggatctcagtcacgcttcgaggctgccaaaatcatccttcagaagatcgatgaataccgaagcggcaaatttgggacacttatcgcaggggtattcctcgcaggtgattttaatagtcaagagacgcaagaagcctataatgtcttgacagggtcggaatcctccttggtcgacacagcaaaggttgtggaacctagccagcactacggcaattattatacatggacagggttcggatatgagggagaggatcccacgcgcattgattatattttaatcggacctgggaagaacaaacttgggtcctggatagtcaacggatacgccgtgttagcgaaccgattcgattctggcgtgtttctgtcagaccacagggccgttgtcgcagacatcaccttgtacaactga

SEQ ID NO: 27 - protein, 300 aa, Aspergillus fumigates
MKTTTELPLRILTHNIRYATSSPFKGELPWNDRKQPLLNELLFNTRNQDAFICLQEVLHNQLVDVLSGLKQPPSTIPKSASETQQWEYIGVGRDDGHKAGEYSPIFYQPSVWQLCHWESVWLSETPNKPSKGWDAASIRILTIGVFTHNITRHTVLVMNTHLDDQGSQSRFEAAKIILQKIDEYRSGKFGTLIAGVFLAGDFNSQETQEAYNVLTGSESSLVDTAKVVEPSQHYGNYYTWTGFGYEGEDPTRIDYILIGPGKNKLGSWIVNGYAVLANRFDSGVFLSDHRAVVADITLYN

SEQ ID NO: 28 - DNA, 1461 bp, Aspergillus fumigates
atgtcgctctcaccgcgagaattgttcgccatatcgacgacagaacgcatatgctctgcgatctctctcgctggcacaagtatcataatcatctctttcctctcatctagatcctttcgcaagccgatcaatcgtctcgtattttacgcgtcatggggaaatatcatggcaaatgtagccacgatgatctctcagtcgggcatcgcctacgggacaagcagctgcctttgtcagttccaggcgtttctgattcaatggtcagtagctcggtcggattgcccgtgtctgatgcccatgacttttatactgatggttgctgttcgcatgtcgcaggttcatgcccgctgatgcgttgtggactcttgccatggcatgcaatgtctacttgacattcttccacaaatataactcggaacagctgcgacaactcgagtggaaatacgtgcttttctgctatggcctcccctttattccggcattcgtttatttcttcatagaaaccgaggctcggggaaaggtttacggctcagccatagtaagtgcaacgtttgtcgtaggggaatcagtcatatgcagagttaccctctaactcgttgttgcttgacagctctggtgctgggtgtcgctcccctgggattttcttcgcattgcggtcttttatggtccagtctggttcgtcattttcctaacatttgccatttacctgcgtgctggcaaagtaatcttcgagaaacgacggcaactcgaggaggctgagtgtccggagtcttccggcgaaattgacagtcctatggagcccgctgtttccaaaaagacagaaatccacgtcaccagcgagatcacacattctgggagtgaatccaatcgattgtccatcatgagtgccagccttataccgcatcgatacctcagcccatactcgccctactctgtaaccattgaaggtggcagcgcagctggcaacgacgaacatgtgccaatgcggacgttgagaagcagccagcacgatccatacgctcagcatcacgctatggcgagagacgtgaactcggccgcctgggcttataccaaatatgcgatgcttttcttcatcgcactgcttgtcacatgggtcagttcatcatcatcagcagcagcagaagcaatggtgtcagtaacagtctggactgatattgatatttggaataggtaccctccacgattaacagattgtacgcgctgatctatccgcgcaacttcaacttcggcatgaactatacgtccagtttcgtccttccgctgcaaggcttctggaatagtatcatttacgtatcaatctcttggcctgctttcaaagaggcttttgcgaagatcaaatggcgacattccctgcaaagaggcccatcccaacacataatcactggacatggggctgtcggaagtctccattcgcatgggaacgacagcacccgcgcgttgacg

SEQ ID NO: 29 - DNA, 1236 bp, Aspergillus fumigates
atgtcgctctcaccgcgagaattgttcgccatatcgacgacagaacgcatatgctctgcgatctctctcgctggcacaagtatcataatcatctctttcctctcatctagatcctttcgcaagccgatcaatcgtctcgtattttacgcgtcatggggaaatatcatggcaaatgtagccacgatgatctctcagtcgggcatcgcctacgggacaagcagctgcctttgtcagttccaggcgtttctgattcaatggttcatgcccgctgatgcgttgtggactcttgccatggcatgcaatgtctacttgacattcttccacaaatataactcggaacagctgcgacaactcgagtggaaatacgtgcttttctgctatggcctcccctttattccggcattcgtttatttcttcatagaaaccgaggctcggggaaaggtttacggctcagccatactctggtgctgggtgtcgctcccctgggattttcttcgcattgcggtcttttatggtccagtctggttcgtcattttcctaacatttgccatttacctgcgtgctggcaaagtaatcttcgagaaacgacggcaactcgaggaggctgagtgtccggagtcttccggcgaaattgacagtcctatggagcccgctgtttccaaaaagacagaaatccacgtcaccagcgagatcacacattctgggagtgaatccaatcgattgtccatcatgagtgccagccttataccgcatcgatacctcagcccatactcgccctactctgtaaccattgaaggtggcagcgcagctggcaacgacgaacatgtgccaatgcggacgttgagaagcagccagcacgatccatacgctcagcatcacgctatggcgagagacgtgaactcggccgcctgggcttataccaaatatgcgatgcttttcttcatcgcactgcttgtcacatgggtaccctccacgattaacagattgtacgcgctgatctatccgcgcaacttcaacttcggcatgaactatacgtccagtttcgtccttccgctgcaaggcttctggaatagtatcatttacgtatcaatctcttggcctgctttcaaagaggcttttgcgaagatcaaatggcgacattccctgcaaagaggcccatcccaacacataatcactggacatggggctgtcggaagtctccattcgcatgggaacgacagcacccgcgcgttgacg

SEQ ID NO: 30 - protein, 111 aa, Aspergillus fumigates
MSLSPRELFAISTTERICSAISLAGTSIIIISFLSSRSFRKPINRLVFYASWGNIMANVATMISQSGIAYGTSSCLCQFQAFLIQWFMPADALWTLAMACNVYLTFFHKYN

SEQ ID NO: 31 - DNA, 1803 bp, Aspergillus fumigates
atggagactggcaccgacaaaggctcggcggacgccatcctcgagcatctaggctatactcccgaactgtcccgcaaccggtccgtcctgcaggtcgcctttatgtgctttatcctctcctccgtcccctacggtctggctaccaccttcttctacccactcgccgccggaggcccgtccaccatcgtctggggctggattatcgtctccctcgttatcctgtgtgtagccatctcgctggccgagatcacctccgtctatcccactgccggtggcgtctactaccagacctttgccctatcgcccccctggtgccgtcgcgccgccgcctggatctgtggatgggcctatgtcctcggcaatatcaccattaccctggctgtaaactttgggaccaccctgttctttgtcgcctgtctcaatgtcttcgaatccgagcccggtgtcgggattgtagatgacatgcagacctaccagatctacctcatcttcctcgccatcaccctgttgacccatgcgatctcgtccctaggcaacaaatggctccccagtctggaggtatgtttcttttcttttcttttcttttttttttttatttattttccaatgttttccctccgttgcgacccgtgtgtcttggctcccaatgcgacttctcatgcgtttcgtctccccatatagatttcagccatcttcttgaccctcatcggcctgattgccctgatcatctccgtccttgtggtcgccaaacatggtcgacactctggtaagtgggtcttcgccgactttgagccccagtcgggctggccggccggctggtctttctgtatcggtctgttgcaggccgcatacgccacctcggcgactggcatgatcatctcgtgagcaatcccgtttcgcccttttgcatgatcggtccattaagattttgataggatgtgcgaggaagtccgggagcctgccattcaggtgcccaaagccatggtgggcaccatcgtgctcaacttcgtggccggcttgggcttcttgctgccgctgacctttgtcctgcccgatatcacgatgctggtgaacctggcctcggggcagcccacccccgtgatcctgaaagatgcgctgggcagctcgaccggagccttcctgctgcttctccctctgctcattctcggcgtcatctgcggcgtgggatgtgtgacggccgcctcccgctgcacctgggcgtttgcgcgcgacggcggtatccccggctccaagtggtggaagaccgtcaacgcgacgctggacattcccctcaacgccatgatgctgggcatgacggtggagattgccctcggtgccatctactttggctccacggccgcgttcaacgccttctccggtgtcggtgtgattttcctgaccctcagctacgcgtgccccatcgcggtctcgttcttcttccgtcgtcgctcggagatcgccaacgccagattcaacctggggatcatcggcagtatctgcaacgtggttgctctgggtaagtctccgccccgctcgcccactccgcaactcactcctcctgggcaacctactgacgggtctacagcatggagtcttctggccatccctctgttctgtatgccgacgtacaaggtcgtcaccctggagaccatgaactacgcctgtgtcgtcttcgtcgggttcacgaccatcgccggactctggtacttggtctggggctaccgcaactacgacggtccccccaaggagggtatcgatggagtggaggccgatttccccgatctgcccgccaagtctgggtaa

SEQ ID NO: 32 - DNA, 1557 bp, Aspergillus fumigates
atggagactggcaccgacaaaggctcggcggacgccatcctcgagcatctaggctatactcccgaactgtcccgcaaccggtccgtcctgcaggtcgcctttatgtgctttatcctctcctccgtcccctacggtctggctaccaccttcttctacccactcgccgccggaggcccgtccaccatcgtctggggctggattatcgtctccctcgttatcctgtgtgtagccatctcgctggccgagatcacctccgtctatcccactgccggtggcgtctactaccagacctttgccctatcgcccccctggtgccgtcgcgccgccgcctggatctgtggatgggcctatgtcctcggcaatatcaccattaccctggctgtaaactttgggaccaccctgttctttgtcgcctgtctcaatgtcttcgaatccgagcccggtgtcgggattgtagatgacatgcagacctaccagatctacctcatcttcctcgccatcaccctgttgacccatgcgatctcgtccctaggcaacaaatggctccccagtctggagatttcagccatcttcttgaccctcatcggcctgattgccctgatcatctccgtccttgtggtcgccaaacatggtcgacactctggtaagtgggtcttcgccgactttgagccccagtcgggctggccggccggctggtctttctgtatcggtctgttgcaggccgcatacgccacctcggcgactggcatgatcatctcgatgtgcgaggaagtccgggagcctgccattcaggtgcccaaagccatggtgggcaccatcgtgctcaacttcgtggccggcttgggcttcttgctgccgctgacctttgtcctgcccgatatcacgatgctggtgaacctggcctcggggcagcccacccccgtgatcctgaaagatgcgctgggcagctcgaccggagccttcctgctgcttctccctctgctcattctcggcgtcatctgcggcgtgggatgtgtgacggccgcctcccgctgcacctgggcgtttgcgcgcgacggcggtatccccggctccaagtggtggaagaccgtcaacgcgacgctggacattcccctcaacgccatgatgctgggcatgacggtggagattgccctcggtgccatctactttggctccacggccgcgttcaacgccttctccggtgtcggtgtgattttcctgaccctcagctacgcgtgccccatcgcggtctcgttcttcttccgtcgtcgctcggagatcgccaacgccagattcaacctggggatcatcggcagtatctgcaacgtggttgctctggcatggagtcttctggccatccctctgttctgtatgccgacgtacaaggtcgtcaccctggagaccatgaactacgcctgtgtcgtcttcgtcgggttcacgaccatcgccggactctggtacttggtctggggctaccgcaactacgacggtccccccaaggagggtatcgatggagtggaggccgatttccccgatctgcccgccaagtctgggtaa

SEQ ID NO: 33 - protein, 518 aa, Aspergillus fumigates
METGTDKGSADAILEHLGYTPELSRNRSVLQVAFMCFILSSVPYGLATTFFYPLAAGGPSTIVWGWIIVSLVILCVAISLAEITSVYPTAGGVYYQTFALSPPWCRRAAAWICGWAYVLGNITITLAVNFGTTLFFVACLNVFESEPGVGIVDDMQTYQIYLIFLAITLLTHAISSLGNKWLPSLEISAIFLTLIGLIALIISVLVVAKHGRHSGKWVFADFEPQSGWPAGWSFCIGLLQAAYATSATGMIISMCEEVREPAIQVPKAMVGTIVLNFVAGLGFLLPLTFVLPDITMLVNLASGQPTPVILKDALGSSTGAFLLLLPLLILGVICGVGCVTAASRCTWAFARDGGIPGSKWWKTVNATLDIPLNAMMLGMTVEIALGAIYFGSTAAFNAFSGVGVIFLTLSYACPIAVSFFFRRRSEIANARFNLGIIGSICNVVALAWSLLAIPLFCMPTYKVVTLETMNYACVVFVGFTTIAGLWYLVWGYRNYDGPPKEGIDGVEADFPDLPAKSG

SEQ ID NO: 34 - DNA, 1971 bp, Aspergillus fumigates
atgaagacaaaagcaacctccctcttcctctgcgtctcggcgcttgtatcctcaatctctgccctcaccatcagccgaatcaatggaaacaaatacctctcgccctacgccggcgaaaccgtctccaatatccaaggcctggtaacagccaaaggcccctctggcttttacctacggtcaacaaccccagacgacgacgacgccacctcggaatccatctacgtctacggcagcaccgctgtctccaaagtctccgtcggcgacatcatctccctttcggggaaagtctccgagtaccgctcctcggcctcatacgtgtatctaacggaactcacctcgccatctgccatctccgtcgtctcgcgcgggaatgccgtcgtgcccgtggtggtcggcaagggcggccgggcccctccgacggagcagttctccgtgctggacggcggggatgtgcttgctgtgccgaataatgtgagcacagtcagcaagacgaatccagtgctgcggccggggacgtacgggatggacttttgggagagtctgagcggggagttggtgagggtgtcgaacgtaagggcgattgcgaggccgaattcgtacggtgatacgtgggtgcgtggggattggaaggtaagtggggagaatgagaggggtgggttgacgatgcgggagaagggtatgcatgcatgttcgtcaggatgtttagacgaggctgatgaacgtacctagactcgaaccccgaggctgtcattgttgggtcgccgctggatggtagtaaaaatcctactgataccaagctgggggatttggttggggatatcacaggcgtgatcacgacggcgtacgggtactatgtgctgctgcccctgacagccttgactgttacggggtcaaacacgacggctgctgctcctacagatttggagtctgctggaacatgcggtggtgtgacggttggctcgtacaatgtcaacaacctggctcccaactcgacgacgctgcccaagatcgcgcagcatattgcgcagtacctgaagagtccgacgctggtctttctgcaagaaatccaggatgatgacgggccgactgatgacggcggtatgcttcctctcaggctgactgtggccatcggctaatgcatgcgccagtggtatccgccaacaagacgctctcgaccctcgtgcagtccatcgccgaccaaggcggcatccggtactcgtttgtcgatattgccccggtcaacaaggaagacggcggccagccaggcgggaatatccgcgtcgcctacctgtacgatccgtctgtgattcgcctgcgtgacgccaaccctggctccaacaccgatgccaatgaggtcctccccggccccgaactcaagtataaccctggcctgatcgacccgtccaacaacgcgtggctcaacagtcgcaagccgctagccgcagcctgggaaacgctcgacggcaagaacaagttcttcacggtgaatgtgcacttcaccagtaagggcggtggatcgtcaattgagggcgatgcccggccgccagtcaacggaggcgttgccacacgcgaggcgcaggccaagctcgttgctgtacgttgtcctcgttctcaatccttgagcggtactaacgtcagaaggaatttacatcgtccatcctagccgaagactccaccgccaagatcattgtttccggcgactttaacgagttcacctttgcacagccgcttgaaacgttcctggctgaatctgggttggaggatctcgacgaggtcgctgggattgcagccacggagcggtacacgtatctgtacgatatgaactgccaacagctggaccacatgtttgtgagcccggcgctggcaacaggagcgcagatgcaccatctgcatgtcaacacctgggtttcgttcgacgaccaggcaagcgaccatgatcctacggtggcgttgctgaacgtgtgtagctag

SEQ ID NO: 35 - DNA, 1824 bp, Aspergillus fumigates
atgaagacaaaagcaacctccctcttcctctgcgtctcggcgcttgtatcctcaatctctgccctcaccatcagccgaatcaatggaaacaaatacctctcgccctacgccggcgaaaccgtctccaatatccaaggcctggtaacagccaaaggcccctctggcttttacctacggtcaacaaccccagacgacgacgacgccacctcggaatccatctacgtctacggcagcaccgctgtctccaaagtctccgtcggcgacatcatctccctttcggggaaagtctccgagtaccgctcctcggcctcatacgtgtatctaacggaactcacctcgccatctgccatctccgtcgtctcgcgcgggaatgccgtcgtgcccgtggtggtcggcaagggcggccgggcccctccgacggagcagttctccgtgctggacggcggggatgtgcttgctgtgccgaataatgtgagcacagtcagcaagacgaatccagtgctgcggccggggacgtacgggatggacttttgggagagtctgagcggggagttggtgagggtgtcgaacgtaagggcgattgcgaggccgaattcgtacggtgatacgtgggtgcgtggggattggaaggtaagtggggagaatgagaggggtgggttgacgatgcgggagaaggactcgaaccccgaggctgtcattgttgggtcgccgctggatggtagtaaaaatcctactgataccaagctgggggatttggttggggatatcacaggcgtgatcacgacggcgtacgggtactatgtgctgctgcccctgacagccttgactgttacggggtcaaacacgacggctgctgctcctacagatttggagtctgctggaacatgcggtggtgtgacggttggctcgtacaatgtcaacaacctggctcccaactcgacgacgctgcccaagatcgcgcagcatattgcgcagtacctgaagagtccgacgctggtctttctgcaagaaatccaggatgatgacgggccgactgatgacggcgtggtatccgccaacaagacgctctcgaccctcgtgcagtccatcgccgaccaaggcggcatccggtactcgtttgtcgatattgccccggtcaacaaggaagacggcggccagccaggcgggaatatccgcgtcgcctacctgtacgatccgtctgtgattcgcctgcgtgacgccaaccctggctccaacaccgatgccaatgaggtcctccccggccccgaactcaagtataaccctggcctgatcgacccgtccaacaacgcgtggctcaacagtcgcaagccgctagccgcagcctgggaaacgctcgacggcaagaacaagttcttcacggtgaatgtgcacttcaccagtaagggcggtggatcgtcaattgagggcgatgcccggccgccagtcaacggaggcgttgccacacgcgaggcgcaggccaagctcgttgctaaggaatttacatcgtccatcctagccgaagactccaccgccaagatcattgtttccggcgactttaacgagttcacctttgcacagccgcttgaaacgttcctggctgaatctgggttggaggatctcgacgaggtcgctgggattgcagccacggagcggtacacgtatctgtacgatatgaactgccaacagctggaccacatgtttgtgagcccggcgctggcaacaggagcgcagatgcaccatctgcatgtcaacacctgggtttcgttcgacgaccaggcaagcgaccatgatcctacggtggcgttgctgaacgtgtgtagctag

SEQ ID NO: 36 - protein, 606 aa, Aspergillus fumigates
MKTKATSLFLCVSALVSSISALTISRINGNKYLSPYAGETVSNIQGLVTAKGPSGFYLRSTTPDDDDATSESIYVYGSTAVSKVSVGDIISLSGKVSEYRSSASYVYLTELTSPSAISVVSRGNAVVPWVGKGGRAPPTEQFSVLDGGDVLAVPNNVSTVSKTNPVLRPGTYGMDFWESLSGELVRVSNVRAIARPNSYGDTWVRGDWKVSGENERGGLTMREKDSNPEAVIVGSPLDGSKNPTDTKLGDLVGDITGVITTAYGYYVLLPLTALTVTGSNTTAAAPTDLESAGTCGGVTVGSYNVNNLAPNSTTLPKIAQHIAQYLKSPTLVFLQEIQDDDGPTDDGVVSANKTLSTLVQSIADQGGIRYSFVDIAPVNKEDGGQPGGNIRVAYLYDPSVIRLRDANPGSNTDANEVLPGPELKYNPGLIDPSNNAWLNSRKPLAAAWETLDGKNKFFTVNVHFTSKGGGSSIEGDARPPVNGGVATREAQAKLVAKEFTSSILAEDSTAKIIVSGDFNEFTFAQPLETFLAESGLEDLDEVAGIAATERYTYLYDMNCQQLDHMFVSPALATGAQMHHLHVNTWVSFDDQASDHDPTVALLNVCS

SEQ ID NO: 37 - DNA, 1999 bp, Aspergillus nidulans
atggattccacgaaacctacagacaacccttcgctccaggatcccaaatacatcgagtttcctgcccttccgagcgatgcaaagcatgcagatggtacccttgcactgaatcggcactcaacacatataacccgtggtcatgatttccctggtgcaaaggtttgtctaggataccgagcctgcgggtttcatggactaactggcatgcattcttaggccatgctttatgctgccggcgtcccggataaggagtctatggcgaagagtccccatgttggaatcgcaggttcaacctcctcaacccatttcgcagactttccgctaactgataaggtgtttggtgggagggaaacccatgcaatatgcacctcctggaccttgctacgaccgctaagaaagccgtgattgaccggggtatgcttggatggcaatacaataccattggagtttcagacgcaatctctatgggtagtgaaggtacgctcttagactggcatatccaaaccggtaccgcgttgtggaagtggaggcttattattgcgcaggcatgagattctcgcttcaatcccgtgaaatcattgccgatagtgtcgaaaccgttacgtgtgcgcaatatcacgatgcctgtatcgccattccgggttgtgataagaacatgcccggtgttgttatgggtatggccagacataaccgaccgtccctgatgatatacggtggtacaattcagaagggttactcgcagtcacttcggaagaacatcagcgtgtcttcgtgcttcgaagctgcgggcgcatacgcatatgataccctgcgccagcctgacgatggaggcgacacgagcctaacaaaggacgaaatcatggacgatctggaaaagcacgcttgtcctagcgcaggtgcatgtggaggaatgtttaccgcaaacacaatggccaccgcaattgaatccatggggttgactctgccgggttcatcgtcgacgcctgcttcgtcgccgaccaagatgcgagaatgtgtcaaggcagccgatgctattaaaacctgcctggagaaaaacattcgtccgcgtgacttgctcaccaaaaggtcctttgagaacgctcttgttatgactatggcattaggaggaagcaccaatggtgtgcttcatttcctggctatggccagaaccgcgggcgttgatctcaccttggatgacgtccaaagagttagcaacaagattccattcattgcagacctttccccaagtggaaaatactttatggccgacctttacgaaatcggaggcattccctccgtccataagctgttgattgcggctggcctcatcgacggtggtattcccaccgtaaccggcaagactctagctgaaaatgtggcctcatatccatctcttcccgacgatcaggtcattatccgtcctttgaacaaccccatcaagccgactggtcatctccagatcctaaagggtaatcttgctccaggtggcgcggtggccaagattaccggcaaagaaggaaccaaattcaccggaaaagcgcgcgttttcgacaaggagtaccagcttaacgatgccttgacgcagggcaagatcccccgcggagaaaaccttgtgctcatcgttcgctacgaaggtccaaagggaggaccaggtatgccagaacagctcaaagccagtgcggcgcttatgggtgcaaaactgaccaacgtggcattgattacggatggtcggtactctggggcctcgcatggctttatcgttgggcatattgtgccagaggcagcagtgggaggccccattgccgttgttcgtgacggggactctgtcactatcaacgcggagaccaacgaactcagcatggatgtttcggatgaggagattcaacggcggttgaaagaatggaagccaccggctcctactgtgacgcgcggtgtgcttgcgaagtatgcccggctggtaggggatgcttcgcacggtgccatgacagatctgttttga

SEQ ID NO: 38 - DNA, 1827 bp, Aspergillus nidulans
atggattccacgaaacctacagacaacccttcgctccaggatcccaaatacatcgagtttcctgcccttccgagcgatgcaaagcatgcagatggtacccttgcactgaatcggcactcaacacatataacccgtggtcatgatttccctggtgcaaaggccatgctttatgctgccggcgtcccggataaggagtctatggcgaagagtccccatgttggaatcgcaggtgtttggtgggagggaaacccatgcaatatgcacctcctggaccttgctacgaccgctaagaaagccgtgattgaccggggtatgcttggatggcaatacaataccattggagtttcagacgcaatctctatgggtagtgaaggcatgagattctcgcttcaatcccgtgaaatcattgccgatagtgtcgaaaccgttacgtgtgcgcaatatcacgatgcctgtatcgccattccgggttgtgataagaacatgcccggtgttgttatgggtatggccagacataaccgaccgtccctgatgatatacggtggtacaattcagaagggttactcgcagtcacttcggaagaacatcagcgtgtcttcgtgcttcgaagctgcgggcgcatacgcatatgataccctgcgccagcctgacgatggaggcgacacgagcctaacaaaggacgaaatcatggacgatctggaaaagcacgcttgtcctagcgcaggtgcatgtggaggaatgtttaccgcaaacacaatggccaccgcaattgaatccatggggttgactctgccgggttcatcgtcgacgcctgcttcgtcgccgaccaagatgcgagaatgtgtcaaggcagccgatgctattaaaacctgcctggagaaaaacattcgtccgcgtgacttgctcaccaaaaggtcctttgagaacgctcttgttatgactatggcattaggaggaagcaccaatggtgtgcttcatttcctggctatggccagaaccgcgggcgttgatctcaccttggatgacgtccaaagagttagcaacaagattccattcattgcagacctttccccaagtggaaaatactttatggccgacctttacgaaatcggaggcattccctccgtccataagctgttgattgcggctggcctcatcgacggtggtattcccaccgtaaccggcaagactctagctgaaaatgtggcctcatatccatctcttcccgacgatcaggtcattatccgtcctttgaacaaccccatcaagccgactggtcatctccagatcctaaagggtaatcttgctccaggtggcgcggtggccaagattaccggcaaagaaggaaccaaattcaccggaaaagcgcgcgttttcgacaaggagtaccagcttaacgatgccttgacgcagggcaagatcccccgcggagaaaaccttgtgctcatcgttcgctacgaaggtccaaagggaggaccaggtatgccagaacagctcaaagccagtgcggcgcttatgggtgcaaaactgaccaacgtggcattgattacggatggtcggtactctggggcctcgcatggctttatcgttgggcatattgtgccagaggcagcagtgggaggccccattgccgttgttcgtgacggggactctgtcactatcaacgcggagaccaacgaactcagcatggatgtttcggatgaggagattcaacggcggttgaaagaatggaagccaccggctcctactgtgacgcgcggtgtgcttgcgaagtatgcccggctggtaggggatgcttcgcacggtgccatgacagatctgttttga

SEQ ID NO: 39 - protein, 608 aa, Aspergillus nidulans
MDSTKPTDNPSLQDPKYIEFPALPSDAKHADGTLALNRHSTHITRGHDFPGAKAMLYAAGVPDKESMAKSPHVGIAGVWWEGNPCNMHLLDLATTAKKAVIDRGMLGWQYNTIGVSDAISMGSEGMRFSLQSREIIADSVETVTCAQYHDACIAIPGCDKNMPGVVMGMARHNRPSLMIYGGTIQKGYSQSLRKNISVSSCFEAAGAYAYDTLRQPDDGGDTSLTKDEIMDDLEKHACPSAGACGGMFTANTMATAIESMGLTLPGSSSTPASSPTKMRECVKAADAIKTCLEKNIRPRDLLTKRSFENALVMTMALGGSTNGVLHFLAMARTAGVDLTLDDVQRVSNKIPFIADLSPSGKYFMADLYEIGGIPSVHKLLIAAGLIDGGIPTVTGKTLAENVASYPSLPDDQVIIRPLNNPIKPTGHLQILKGNLAPGGAVAKITGKEGTKFTGKARVFDKEYQLNDALTQGKIPRGENLVLIVRYEGPKGGPGMPEQLKASAALMGAKLTNVALITDGRYSGASHGFIVGHIVPEAAVGGPIAVVRDGDSVTINAETNELSMDVSDEEIQRRLKEWKPPAPTVTRGVLAKYARLVGDASHGAMTDLF

SEQ ID NO: 40 - DNA, 1983 bp, Aspergillus nidulans
atgcttctttcacagactcggggccgcctgccctctgctctccgcagcttggccaacagagctgctatgtaggtcacctcccattgtacagctaatcaatggttttacgtcttggttgtacgtctgctaagtgaaaatgcgtccagtcgtccaatctctactacactcccccgccaaaaagcgtcgccaaaagatgatgagcccgtcctcaacaaggtctcccgccatatcacacagccggtgtcccagggtgcttcccaggcgatgctgtacgctacgggtcttactgaggccgacatgaacaaggcccaggttggtatttcctcggtctggtacaacggcaacccttgcaacatgcacctcctcgacctgaacaaccgtgtccgcgagggtgtgcaaaaggctggccttatcgggtaccagttcaacaccattggtgtcagtgatggaatcagtatgggtaccagtggtatgcgttactcgcttcagagccgtgacctcatcgccgactctatcgagaccgtcatgggtggtcagtggtacgacgccaacatcagcatccccggttgcgacaagaacatgcccggtgttttgatggctatgggacgagtcaaccgccccagtttgatggtttacggtggaaccatcaagcccggctgcgcatccatgcagggcaacgctgacatcgatatcgtctctgccttccaagcctacggccagttcatcagcggtgagatcaacgagccccagcgcttcgatatcatccgccacgcctgccccggtggcggcgcttgcggtggaatgtacactgccaacaccatggccacggccatcgaagtcatgggtatgaccctcacgggctcctcgtccaacccggccgaatcgcaagccaaatacgacgaatgtcttcgcgctggtgaagccatcaagcgcctcctcgtcgaagacatccgcccctccgacatcatgactcggcaagccttcgagaacgccatggtcgttgtcaacatcaccggcggctccaccaatgctgtccttcacctcatcgccattgccgactccgtcggcatcaagctcacaattgacgacttccaagccgtctctgaccgcacccccttcctcgcagacctcaagccatccggcaaatacgttatggccgacctccacaacatcggcggcaccccctccctcctcaaattcctcctcaaggaaggcgtcattgatggctccggcatcacagttaccggtgaaactctcgccaagaacctcgagaaagtccccgatttccccgaggaccagaaaatcattcgccccttctccaaccccatcaaggaaacaggccacatccagatcctgcgcggttcgctcgcgccgggcggttgcgttggtaagattaccggtaaggagggaaccgttttcacgggcaaggcccgcgtctttaaccacgaagacgacttcattgccgccctggagcgcaaggaaatcaccaaggatgagcaaactgtcgttgtgattcgctacaccggtcccaagggtggtcccggtatgcctggtatgcactctctagcaacctgacccttcaattctcatccaacctgctaattcacctttctagaaatgctcaagccttcaagcgccctcatgggtgctggcctcggccaaacctgcgccctgatcacagacggacgcttctccggtggttcgcacggcttccttattggacacatcgtcccagaggctgccgtcggtgggccgattggtctcgtacacgacggcgacgtgatcaccattgatgccgagaagcgcgttttggaccttgacgttgacgaggcggaactcgctaagcgacggaagcagtgggaggctgataaggcggcgggcaagttgccccagaccgggttgaacttgcgcgggacgcttggaaagtatgcccggaatgtcaaggatgctagttccgggtgtattacggacgctttcgattaa

SEQ ID NO: 41 - DNA, 1842 bp, Aspergillus nidulans
atgcttctttcacagactcggggccgcctgccctctgctctccgcagcttggccaacagagctgctattcgtccaatctctactacactcccccgccaaaaagcgtcgccaaaagatgatgagcccgtcctcaacaaggtctcccgccatatcacacagccggtgtcccagggtgcttcccaggcgatgctgtacgctacgggtcttactgaggccgacatgaacaaggcccaggttggtatttcctcggtctggtacaacggcaacccttgcaacatgcacctcctcgacctgaacaaccgtgtccgcgagggtgtgcaaaaggctggccttatcgggtaccagttcaacaccattggtgtcagtgatggaatcagtatgggtaccagtggtatgcgttactcgcttcagagccgtgacctcatcgccgactctatcgagaccgtcatgggtggtcagtggtacgacgccaacatcagcatccccggttgcgacaagaacatgcccggtgttttgatggctatgggacgagtcaaccgccccagtttgatggtttacggtggaaccatcaagcccggctgcgcatccatgcagggcaacgctgacatcgatatcgtctctgccttccaagcctacggccagttcatcagcggtgagatcaacgagccccagcgcttcgatatcatccgccacgcctgccccggtggcggcgcttgcggtggaatgtacactgccaacaccatggccacggccatcgaagtcatgggtatgaccctcacgggctcctcgtccaacccggccgaatcgcaagccaaatacgacgaatgtcttcgcgctggtgaagccatcaagcgcctcctcgtcgaagacatccgcccctccgacatcatgactcggcaagccttcgagaacgccatggtcgttgtcaacatcaccggcggctccaccaatgctgtccttcacctcatcgccattgccgactccgtcggcatcaagctcacaattgacgacttccaagccgtctctgaccgcacccccttcctcgcagacctcaagccatccggcaaatacgttatggccgacctccacaacatcggcggcaccccctccctcctcaaattcctcctcaaggaaggcgtcattgatggctccggcatcacagttaccggtgaaactctcgccaagaacctcgagaaagtccccgatttccccgaggaccagaaaatcattcgccccttctccaaccccatcaaggaaacaggccacatccagatcctgcgcggttcgctcgcgccgggcggttgcgttggtaagattaccggtaaggagggaaccgttttcacgggcaaggcccgcgtctttaaccacgaagacgacttcattgccgccctggagcgcaaggaaatcaccaaggatgagcaaactgtcgttgtgattcgctacaccggtcccaagggtggtcccggtatgcctgaaatgctcaagccttcaagcgccctcatgggtgctggcctcggccaaacctgcgccctgatcacagacggacgcttctccggtggttcgcacggcttccttattggacacatcgtcccagaggctgccgtcggtgggccgattggtctcgtacacgacggcgacgtgatcaccattgatgccgagaagcgcgttttggaccttgacgttgacgaggcggaactcgctaagcgacggaagcagtgggaggctgataaggcggcgggcaagttgccccagaccgggttgaacttgcgcgggacgcttggaaagtatgcccggaatgtcaaggatgctagttccgggtgtattacggacgctttcgattaa

SEQ ID NO: 42 - protein, 613 aa, Aspergillus nidulans
MLLSQTRGRLPSALRSLANRAAIRPISTTLPRQKASPKDDEPVLNKVSRHITQPVSQGASQAMLYATGLTEADMNKAQVGISSVWYNGNPCNMHLLDLNNRVREGVQKAGLIGYQFNTIGVSDGISMGTSGMRYSLQSRDLIADSIETVMGGQWYDANISIPGCDKNMPGVLMAMGRVNRPSLMVYGGTIKPGCASMQGNADIDIVSAFQAYGQFISGEINEPQRFDIIRHACPGGGACGGMYTANTMATAIEVMGMTLTGSSSNPAESQAKYDECLRAGEAIKRLLVEDIRPSDIMTRQAFENAMVVVNITGGSTNAVLHLIAIADSVGIKLTIDDFQAVSDRTPFLADLKPSGKYVMADLHNIGGTPSLLKFLLKEGVIDGSGITVTGETLAKNLEKVPDFPEDQKIIRPFSNPIKETGHIQILRGSLAPGGCVGKITGKEGTVFTGKARVFNHEDDFIAALERKEITKDEQTVVVIRYTGPKGGPGMPEMLKPSSALMGAGLGQTCALITDGRFSGGSHGFLIGHIVPEAAVGGPIGLVHDGDVITIDAEKRVLDLDVDEAELAKRRKQWEADKAAGKLPQTGLNLRGTLGKYARNVKDASSGCITDAFD

SEQ ID NO: 43 - DNA, 1886 bp, Gibberella zeae
atgctttcccggtcactgctgcgctctagagctgtgggagctttccccctctctgccaggaatcatgggtatgtctaccatcatccgttgccaacgatcaacaacatcctgcacaaggccaattcacttctgaatactaacccatcttccattacagccgcttcctgtctaccacctccatccgctccgatgacaagctcaacaagatctcctccaacatcactcagcccaaggcccagggtgcttcccaggctatgctctacgccaccggcctctctgaagctgacatgaacaaggctcaagttggcatctcgtccgtctggtacgagggaaacccttgcaacatgcaccttatggacctctctgcccacgtcaaggagtctgtcgccaaggccggtcttattccctaccgattcaacaccatcggtgtctctgatggtatctccatgggtaccactggtatgcgatattccctccagagccgagagattattgccgatagtgttgagactgtcatgaacggtcaatggtatgacgccaacgtcagcttgcccggttgcgacaagaacatgcccggtgttgccattgccatgggtcgtgtcaaccgtcccagcatcatggtttacggtggtaccattaagcccggttgcaccaagcagggcgagtctatcgatatcgtctctgctttccaggcctacggtcaatacatcaccggcgagatcaccgaggagcagcgattcgacattatccgaaatgcctgccctggtggtggtgcttgtggtggcatgtacactgccaacaccatggctactgccattgagactctgggacttaccctccctggtagcagcagcagccctgctgaggaccccagcaagatcgccgagtgtgaggctgttggacctgctatccgcaacattctcaaggaagatatccgacctcgtgacatcatgactcgccaagcctttgagaatgccatgatcgtcactaccatccttggtggcagcaccaacgctgttctgcatcttatcgcaattgccgactctgtcggtatcaagctcgacatcgaggacttccaaaaggtttccgaccgcactcccttccttgccgacctgaagccctctggaaagtgggtcatggccgatatgcacaagattggtggtactcctgctcttctcaagttcctcttgaaggagggtatcattgacggctctggtatcactgtcactggcaagaccatgaagcagaacgtcgaggagttgcctggattccccgaggatcaaaccatcattcgcccccttagcaaccccattaagcctaccggtcacatccagattctccgtggatccctggctcctggtggctgtgttggtaagatcactggcaaggagggtctccgattcgagggtaaggcccgtgtctacgactccgagcccgccttcatctctagccttgaggctggtgagatcaagaagggtgagaagactgtcgttatcatccgatatgatggacccaagggtggccccggtatgcctgagatgctgaagccttcttctgccattatgggtgctggccttggacaggatgtcgcccttctcactgacggtcgcttctctggtggttctcacggtttcattattggtcacattgtccccgaggcaatggagggtggccctatcgcccttgtcgaggacggtgacaccatcgttatcgacgccgagtctcgtgctatcgacctcgttgttcccgaggcagaggttgatcgccgtcgcaaggcctggaaggctcccgctccccgatacaccaagggcacactcagcaagtacgctcgactggtgaccaacgccagtgagggctgtgtcaccgatagcggtctcaagaactaa

SEQ ID NO: 44 - DNA, 1797 bp, Gibberella zeae
atgctttcccggtcactgctgcgctctagagctgtgggagctttccccctctctgccaggaatcatggccgcttcctgtctaccacctccatccgctccgatgacaagctcaacaagatctcctccaacatcactcagcccaaggcccagggtgcttcccaggctatgctctacgccaccggcctctctgaagctgacatgaacaaggctcaagttggcatctcgtccgtctggtacgagggaaacccttgcaacatgcaccttatggacctctctgcccacgtcaaggagtctgtcgccaaggccggtcttattccctaccgattcaacaccatcggtgtctctgatggtatctccatgggtaccactggtatgcgatattccctccagagccgagagattattgccgatagtgttgagactgtcatgaacggtcaatggtatgacgccaacgtcagcttgcccggttgcgacaagaacatgcccggtgttgccattgccatgggtcgtgtcaaccgtcccagcatcatggtttacggtggtaccattaagcccggttgcaccaagcagggcgagtctatcgatatcgtctctgctttccaggcctacggtcaatacatcaccggcgagatcaccgaggagcagcgattcgacattatccgaaatgcctgccctggtggtggtgcttgtggtggcatgtacactgccaacaccatggctactgccattgagactctgggacttaccctccctggtagcagcagcagccctgctgaggaccccagcaagatcgccgagtgtgaggctgttggacctgctatccgcaacattctcaaggaagatatccgacctcgtgacatcatgactcgccaagcctttgagaatgccatgatcgtcactaccatccttggtggcagcaccaacgctgttctgcatcttatcgcaattgccgactctgtcggtatcaagctcgacatcgaggacttccaaaaggtttccgaccgcactcccttccttgccgacctgaagccctctggaaagtgggtcatggccgatatgcacaagattggtggtactcctgctcttctcaagttcctcttgaaggagggtatcattgacggctctggtatcactgtcactggcaagaccatgaagcagaacgtcgaggagttgcctggattccccgaggatcaaaccatcattcgcccccttagcaaccccattaagcctaccggtcacatccagattctccgtggatccctggctcctggtggctgtgttggtaagatcactggcaaggagggtctccgattcgagggtaaggcccgtgtctacgactccgagcccgccttcatctctagccttgaggctggtgagatcaagaagggtgagaagactgtcgttatcatccgatatgatggacccaagggtggccccggtatgcctgagatgctgaagccttcttctgccattatgggtgctggccttggacaggatgtcgcccttctcactgacggtcgcttctctggtggttctcacggtttcattattggtcacattgtccccgaggcaatggagggtggccctatcgcccttgtcgaggacggtgacaccatcgttatcgacgccgagtctcgtgctatcgacctcgttgttcccgaggcagaggttgatcgccgtcgcaaggcctggaaggctcccgctccccgatacaccaagggcacactcagcaagtacgctcgactggtgaccaacgccagtgagggctgtgtcaccgatagcggtctcaagaactaa

SEQ ID NO: 45 - protein, 598 aa, Gibberella zeae
MLSRSLLRSRAVGAFPLSARNHGRFLSTTSIRSDDKLNKISSNITQPKAQGASQAMLYATGLSEADMNKAQVGISSVWYEGNPCNMHLMDLSAHVKESVAKAGLIPYRFNTIGVSDGISMGTTGMRYSLQSREIIADSVETVMNGQWYDANVSLPGCDKNMPGVAIAMGRVNRPSIMVYGGTIKPGCTKQGESIDIVSAFQAYGQYITGEITEEQRFDIIRNACPGGGACGGMYTANTMATAIETLGLTLPGSSSSPAEDPSKIAECEAVGPAIRNILKEDIRPRDIMTRQAFENAMIVTTILGGSTNAVLHLIAIADSVGIKLDIEDFQKVSDRTPFLADLKPSGKWVMADMHKIGGTPALLKFLLKEGIIDGSGITVTGKTMKQNVEELPGFPEDQTIIRPLSNPIKPTGHIQILRGSLAPGGCVGKITGKEGLRFEGKARVYDSEPAFISSLEAGEIKKGEKTVVIIRYDGPKGGPGMPEMLKPSSAIMGAGLGQDVALLTDGRFSGGSHGFIIGHIVPEAMEGGPIALVEDGDTIVIDAESRAIDLVVPEAEVDRRRKAWKAPAPRYTKGTLSKYARLVTNASEGCVTDSGLKN

SEQ ID NO: 46 - DNA, 2018 bp, Gibberella zeae
atggcggaccaagtcactcacgatcccaagcagtcaagcgactacatccccttcccttgccttcctcccggcggagctctcaaccgttggtctacaaagatcacccgcgagcatgactaccccggagctcaggtgcgttatatctcccaaactcgctgctcgccactgacattgcttgatgtataggctatgctctacggagctggtgtcaaggaccagcacacaatgaagaacgcgccccaggttggtgttgctaccgtctggtggcaaggaaacccgtgcaagtgagtttgaagtcaattgaatatgtacattaagatgagatgctaacgagtttgcgcagtacccatcgtaagtgacatagataaataatttaggaaaacatacttatgaatgatagtccttgatcttggccagatcgtcaagaactccatcgagaaggaaggcatgatcggctggcagttcaacaccgttggtgtatctgacgccatcaccatgggcggcgagggtaagtaaacactcgcaatacataacaatctcactcgctcactcaccccacaggcatgcgcttctctcttcaaactcgagaaatcattgctgattctatcgagtccgtaacctgcgcccagcatcatgacgccaacatctccattcccggctgtgacaagaacatgcccggcacagtcatggccgccgctcgtcacaaccgccccttcatcatgatctacggcggtaccatccgcaagggccactccaacctccttgagaagcccatcaacatcagcacctgctacgaggcctcgggtgctttcaactacggtcgtctgcatgccaagacgaaccccggcgagcctggtcgcgagagctccgatgtcatggatgatatcgagaagcacgcttgtcccggcgccggagcttgtggtggcatgtatacagccaacaccatggctaccgctatcgaggccatgggccttactctgcctggttcatcatcgtaccctgccgagtctcctgagaagcgtcgtgagtgtgagcgcgctgcccaggttatccgaactaccatggagaaggaccttcgtcctcgcgatatcatgacccgtgcctctttcgagaacgctcttgtcctgaccatgattctcggtggttcaacaaacggtgttcttcacttcctcgccatggccaacaccgccgatgttcccctgaccattgacgacatccagcgtgccagtgaccgcacccccttcctcgctgatctcgcccccagtggaaagtactacatggaggatctctacaaggtcggcggtacaccctccgtcatcaagatgctcgtcgcccgtggtcttctcgacggtagcatcatgaccattaccggcaagactctcgccgagaacgtcgcggactggcctagtctggaccccggccaggacatcatccgtcctcttgagaaccccatcaaggactctggccacatccgcatcctaaagggtaactttgcccccggcggcgccgtcgctaagatcactggaaaggagggtctgtccttcaccggcaaggcccgtgtcttcaacaccgagaaggagctcaacggcgcactgaaccgaagcgagatcaagcagtccgatggtaacctcgtcgtcatcgtccgatacgagggccccaagggcggtcccggtatgcctgaacagctcaaggcttctgcagccatcatgggtgccggcctctccaacctggcacttgtcaccgacggacgatacagtggtgcttctcacggtttcatcgtgggccacgttgtgcctgaggctatggtcggaggtcccatcgctctggtcaaggatggagacgagatcactatcgatgcgattaacaaccgaatcgatgtcgacatcactgacgaggagatggagaagcgaaggagtgagtggaagcctcctgcgccccgtgttacgaggggtgtgttggccaagtatgcccgcttggtcggtgatgcttcccacggtgctgtaacagatcagtggtag

SEQ ID NO: 47 - DNA, 1803 bp, Gibberella zeae
atggcggaccaagtcactcacgatcccaagcagtcaagcgactacatccccttcccttgccttcctcccggcggagctctcaaccgttggtctacaaagatcacccgcgagcatgactaccccggagctcaggctatgctctacggagctggtgtcaaggaccagcacacaatgaagaacgcgccccaggttggtgttgctaccgtctggtggcaaggaaacccgtgcaatacccatctccttgatcttggccagatcgtcaagaactccatcgagaaggaaggcatgatcggctggcagttcaacaccgttggtgtatctgacgccatcaccatgggcggcgagggcatgcgcttctctcttcaaactcgagaaatcattgctgattctatcgagtccgtaacctgcgcccagcatcatgacgccaacatctccattcccggctgtgacaagaacatgcccggcacagtcatggccgccgctcgtcacaaccgccccttcatcatgatctacggcggtaccatccgcaagggccactccaacctccttgagaagcccatcaacatcagcacctgctacgaggcctcgggtgctttcaactacggtcgtctgcatgccaagacgaaccccggcgagcctggtcgcgagagctccgatgtcatggatgatatcgagaagcacgcttgtcccggcgccggagcttgtggtggcatgtatacagccaacaccatggctaccgctatcgaggccatgggccttactctgcctggttcatcatcgtaccctgccgagtctcctgagaagcgtcgtgagtgtgagcgcgctgcccaggttatccgaactaccatggagaaggaccttcgtcctcgcgatatcatgacccgtgcctctttcgagaacgctcttgtcctgaccatgattctcggtggttcaacaaacggtgttcttcacttcctcgccatggccaacaccgccgatgttcccctgaccattgacgacatccagcgtgccagtgaccgcacccccttcctcgctgatctcgcccccagtggaaagtactacatggaggatctctacaaggtcggcggtacaccctccgtcatcaagatgctcgtcgcccgtggtcttctcgacggtagcatcatgaccattaccggcaagactctcgccgagaacgtcgcggactggcctagtctggaccccggccaggacatcatccgtcctcttgagaaccccatcaaggactctggccacatccgcatcctaaagggtaactttgcccccggcggcgccgtcgctaagatcactggaaaggagggtctgtccttcaccggcaaggcccgtgtcttcaacaccgagaaggagctcaacggcgcactgaaccgaagcgagatcaagcagtccgatggtaacctcgtcgtcatcgtccgatacgagggccccaagggcggtcccggtatgcctgaacagctcaaggcttctgcagccatcatgggtgccggcctctccaacctggcacttgtcaccgacggacgatacagtggtgcttctcacggtttcatcgtgggccacgttgtgcctgaggctatggtcggaggtcccatcgctctggtcaaggatggagacgagatcactatcgatgcgattaacaaccgaatcgatgtcgacatcactgacgaggagatggagaagcgaaggagtgagtggaagcctcctgcgccccgtgttacgaggggtgtgttggccaagtatgcccgcttggtcggtgatgcttcccacggtgctgtaacagatcagtggtag

SEQ ID NO: 48 - protein, 600 aa, Gibberella zeae
MADQVTHDPKQSSDYIPFPCLPPGGALNRWSTKITREHDYPGAQAMLYGAGVKDQHTMKNAPQVGVATVWWQGNPCNTHLLDLGQIVKNSIEKEGMIGWQFNTVGVSDAITMGGEGMRFSLQTREIIADSIESVTCAQHHDANISIPGCDKNMPGTVMAAARHNRPFIMIYGGTIRKGHSNLLEKPINISTCYEASGAFNYGRLHAKTNPGEPGRESSDVMDDIEKHACPGAGACGGMYTANTMATAIEAMGLTLPGSSSYPAESPEKRRECERAAQVIRTTMEKDLRPRDIMTRASFENALVLTMILGGSTNGVLHFLAMANTADVPLTIDDIQRASDRTPFLADLAPSGKYYMEDLYKVGGTPSVIKMLVARGLLDGSIMTITGKTLAENVADWPSLDPGQDIIRPLENPIKDSGHIRILKGNFAPGGAVAKITGKEGLSFTGKARVFNTEKELNGALNRSEIKQSDGNLVVIVRYEGPKGGPGMPEQLKASAAIMGAGLSNLALVTDGRYSGASHGFIVGHVVPEAMVGGPIALVKDGDEITIDAINNRIDVDITDEEMEKRRSEWKPPAPRVTRGVLAKYARLVGDASHGAVTDQW

SEQ ID NO: 49 - DNA, 813 bp, Magnaporthe oryzae
atgcaacccagcggccgctacatgatggaggacctgtaccgcgtcggaggcaccccgtccgtgctcaagatgcttatcgccgcagggttgatcgacggcacgatcccgacggtgacgggtaaaaccctcgccgagaacgtcgagtcctggccgtccctcgaccccgggcaggacattatccgcccgctctcggaccccgtcaaggccacgggacacattcgcatcctgcgcggcaacctggcccccggcggcgccgtggccaaaatcactggcaaggagggtatttcctttacaggccgggcacgcgtcttcaacaaggagcacgagctggatcacgccctgtccacgagccagatcaaggcgagcgacggcaacctcgtcgtcattgtgcgctacgagggccccaagggcggaccgggcatgcccgagcaactgcgcgcctcggcagccatcatgggcgccggcctctccaacgtcgccctcgtaacggacggccgctacagcggcgccagccacggattcatcgtcggccacgtcgtgcccgaggctgccaccggtgggcccatcgggctggtcaaggacggtgactttgtgcgcatcgatgccgagaccaacaggatcgacattataggcatcgacggcgttgcggccgagggcgatctggacgccgtcgacaaggagctggagaggcgcagggccgagtggaagaagcctgtcatgaagccgctgaggggtgtcctggccaagtatgcgaggttggtcggtgacgcgagccacggtgctgtgacggatcaggaggacccgagctggtga

SEQ ID NO: 50 - protein, 270 aa, Magnaporthe oryzae
MQPSGRYMMEDLYRVGGTPSVLKMLIAAGLIDGTIPTVTGKTLAENVESWPSLDPGQDIIRPLSDPVKATGHIRILRGNLAPGGAVAKITGKEGISFTGRARVFNKEHELDHALSTSQIKASDGNLVVIVRYEGPKGGPGMPEQLRASAAIMGAGLSNVALVTDGRYSGASHGFIVGHVVPEAATGGPIGLVKDGDFVRIDAETNRIDIIGIDGVAAEGDLDAVDKELERRRAEWKKPVMKPLRGVLAKYARLVGDASHGAVTDQEDPSW

SEQ ID NO: 51 - DNA, 1894 bp, Magnaporthe oryzae
atgtcctcaaacctgctgcgcgccagggtgcccaaggccctggctgccaccaggagccatgcgtatgtcattacacagccaattctcgatacagacattgatatatcaaactgtctataccacatcggttgatataacgattagcaaactaacatactccaatgtcaccagggccctcttctccacaacctcgcgccgcgctgagcaactgaacaagacttcggccaagatcacacagcccaagtcccagggtgcgtcacaggccatgctgtacgccaccggcttgaccgaggaggacatgaacaagccgcaggtcggcatctcgtcggtctggtacgagggcaacccctgcaacatgcacatcctcaagctgtcggagcggatccgcgactccgtcaaggccgccaacctggtccccatgcgcttcaacaccattggagtctcggacggtatcagcatgggcacgaccggcatgcgctattcgctgcagagccgtgagatcatcgccgacagtatcgagaccgtcatgcagggccagtggtacgatgccaacatctccatcccgggctgcgacaagaacatgcccggtgttctcatcgccctcggccgtgtcaaccgccccagtttgatcgtttacggcggtaccatcaagcccggctgcagccaaaagggcgagcccatcgacatcgtcagcgccttccaggcctacggccagtacctgactggtgagatcaccgaggagcagcggttcgacatcatccggaacgcgtgccccggcggtggtgcatgcggtggcatgtacactgccaacactttggcgtcggctatcgagactctgggcatgagcttgccgggaagcagcagcaaccccgcagagcaccccagcaagctggccgaatgtgaccaggttggcgaggccatcaagaacatcctccgtgaggacgtgcgcccccgcgacatcatgacgaggcaggcctttgagaacgccatggttgtagtcagcatccttggtggaagcaccaacgccgtcctgcacctgcttgccgttgccgatgcggtcggtgtcaagcttaccattgacgacttccaagccgtttccgaccggacaccgctcctggcagacctcaagccttcgggcaaatacgtcatggaggatgtacacaagattggtggcacgccatctctcctgcgcttcctggccaaggagggcttgatcgatgcttctggcatcaccgtcactggcaagaccatgaaggagaacttggacaagtaccccgacttcccggccgaccagcccatcatccggcctctcagcaaccccctcaagtccacgggccacatccagatcctccgcggtagtctcgcacccggtggctctgtcggcaagattaccggcaaggagggtcttcagttcactggcaaggcccgctgctacgactgcgaggacgactttatcgagtcgctcgagcgtggcgagatcaagaagggtgagaagacggttgtcatcatccgttacgagggccccaagggtggtcccggcatgcccgagatgctgaagcccagctcggccatcatgggtgccggtctcggcaaggacgttgccctcatcactgacggtcgcttctctggcggttcgcacggattcctcatcggacacgttgtgcccgaggccatggagggcggacctatcgcacttgttcgcgatggcgacaccattactattgatgcggagaagcgggtgatcgacacggacgtgtcggacaagaccatggcggagcgccgcgccgagtggaaggctcccccgatcagggagaccaggggaacgctggccaagtacgcagcgctggttagcgacgcgagcagtggttgcgtgacggacaaggtcgcgcgataa

SEQ ID NO: 52 - DNA, 1785 bp, Magnaporthe oryzae
atgtcctcaaacctgctgcgcgccagggtgcccaaggccctggctgccaccaggagccatgcggccctcttctccacaacctcgcgccgcgctgagcaactgaacaagacttcggccaagatcacacagcccaagtcccagggtgcgtcacaggccatgctgtacgccaccggcttgaccgaggaggacatgaacaagccgcaggtcggcatctcgtcggtctggtacgagggcaacccctgcaacatgcacatcctcaagctgtcggagcggatccgcgactccgtcaaggccgccaacctggtccccatgcgcttcaacaccattggagtctcggacggtatcagcatgggcacgaccggcatgcgctattcgctgcagagccgtgagatcatcgccgacagtatcgagaccgtcatgcagggccagtggtacgatgccaacatctccatcccgggctgcgacaagaacatgcccggtgttctcatcgccctcggccgtgtcaaccgccccagtttgatcgtttacggcggtaccatcaagcccggctgcagccaaaagggcgagcccatcgacatcgtcagcgccttccaggcctacggccagtacctgactggtgagatcaccgaggagcagcggttcgacatcatccggaacgcgtgccccggcggtggtgcatgcggtggcatgtacactgccaacactttggcgtcggctatcgagactctgggcatgagcttgccgggaagcagcagcaaccccgcagagcaccccagcaagctggccgaatgtgaccaggttggcgaggccatcaagaacatcctccgtgaggacgtgcgcccccgcgacatcatgacgaggcaggcctttgagaacgccatggttgtagtcagcatccttggtggaagcaccaacgccgtcctgcacctgcttgccgttgccgatgcggtcggtgtcaagcttaccattgacgacttccaagccgtttccgaccggacaccgctcctggcagacctcaagccttcgggcaaatacgtcatggaggatgtacacaagattggtggcacgccatctctcctgcgcttcctggccaaggagggcttgatcgatgcttctggcatcaccgtcactggcaagaccatgaaggagaacttggacaagtaccccgacttcccggccgaccagcccatcatccggcctctcagcaaccccctcaagtccacgggccacatccagatcctccgcggtagtctcgcacccggtggctctgtcggcaagattaccggcaaggagggtcttcagttcactggcaaggcccgctgctacgactgcgaggacgactttatcgagtcgctcgagcgtggcgagatcaagaagggtgagaagacggttgtcatcatccgttacgagggccccaagggtggtcccggcatgcccgagatgctgaagcccagctcggccatcatgggtgccggtctcggcaaggacgttgccctcatcactgacggtcgcttctctggcggttcgcacggattcctcatcggacacgttgtgcccgaggccatggagggcggacctatcgcacttgttcgcgatggcgacaccattactattgatgcggagaagcgggtgatcgacacggacgtgtcggacaagaccatggcggagcgccgcgccgagtggaaggctcccccgatcagggagaccaggggaacgctggccaagtacgcagcgctggttagcgacgcgagcagtggttgcgtgacggacaaggtcgcgcgataa

SEQ ID NO: 53 - protein, 594 aa, Magnaporthe oryzae
MSSNLLRARVPKALAATRSHAALFSTTSRRAEQLNKTSAKITQPKSQGASQAMLYATGLTEEDMNKPQVGISSVWYEGNPCNMHILKLSERIRDSVKAANLVPMRFNTIGVSDGISMGTTGMRYSLQSREIIADSIETVMQGQWYDANISIPGCDKNMPGVLIALGRVNRPSLIVYGGTIKPGCSQKGEPIDIVSAFQAYGQYLTGEITEEQRFDIIRNACPGGGACGGMYTANTLASAIETLGMSLPGSSSNPAEHPSKLAECDQVGEAIKNILREDVRPRDIMTRQAFENAMVVVSILGGSTNAVLHLLAVADAVGVKLTIDDFQAVSDRTPLLADLKPSGKYVMEDVHKIGGTPSLLRFLAKEGLIDASGITVTGKTMKENLDKYPDFPADQPIIRPLSNPLKSTGHIQILRGSLAPGGSVGKITGKEGLQFTGKARCYDCEDDFIESLERGEIKKGEKTVVIIRYEGPKGGPGMPEMLKPSSAIMGAGLGKDVALITDGRFSGGSHGFLIGHVVPEAMEGGPIALVRDGDTITIDAEKRVIDTDVSDKTMAERRAEWKAPPIRETRGTLAKYAALVSDASSGCVTDKVAR

SEQ ID NO: 54 - DNA, 1889 bp, Neurospora crassa
atgctcgctccctcccttctgcgggctcaggcccccagagctctggcttctgcccgtctctccctgtgagttttcagcccagtgacaggcaaccttgatgtccaagcgcaagtgaagctattcaggaagcgatactgaccaaggtctttcctctcccctccagccgctccctctccaccactccccgccgctacaatgcccaggagaagcagctcaacaaggtgtcggccaacatcacccagcccaagtctcagggtgcttcccaggccatgctctacgccaccggcctcaacgaggacgacatgaacaaggcccaggtcggtatctcgtctgtctggtatgagggcaacccttgcaacatgcaccttctcgacctctccggcctcgtcaaggagtccgttgccaaggctggcctcgtccccatgcgcttcaacaccatcggtgtctcggacggtatctccatgggtaccaccggtatgcgctacagcttgcagtcccgtgagattattgccgactccatcgagaccgtcatgaacggccagtggtacgacgccaacatctccctccccggttgcgacaagaacatgcccggtgtcctcatcgccatgggccgtgtcaaccgcccctccatcatggtctacggtggtaccatcaagcccggctgcaacatgaagggcgagaacatcgatatcgtttccgccttccaggcatacggccagtacatctccggtgagatcgacgagaagcagcgcttcgatatcatccgcaacgcctgccctggtggtggcgcctgcggtggcatgtacactgccaacaccatggccaccgccatcgagaccctcggcatgacccttcccggttcctcctcctaccccgccgagtctcccgagaagaagaacgagtgcttgagcgtcggtgaggccatcaagaacctcctccgcgaggacatccgccccaccgatatccttactcgccaggccttcgagaacgccatgatcgtcgtcaacattcttggcggttccaccaacgccgtcctccacttgattgccatcgccgactccgttggcatcaagctcaccatcgatgacttccaggccgtctccgaccgcactcctttccttgccgacctcaagccttccggcaagtgggtcatggaggaccttagcaagattggcggcacccccgcccttctcaaattcctcctcaaggagggaatcctcgacggctccggcatcacctcgaccggtaagaccatgaaggagaacgtcgagaagttccccgacttccccaccgaccaggccatcatccgccctctgtcgaaccccatcaaggaaaccggacacattcagatcctccgcggctctctcgctcccggcggttccgtcggcaagatcaccggcaaggagggtctccgtttcgagggtaaggctcgctgcttcgactacgaggacggctttattgaggctctcgagcgtggcgagatcaagaagggcgagaagaccgtcgtcgtcatccgctacgagggtcccaagggcggccctggcatgcccgaaatgctcaagccttcgtccgccattatgggctacggtctcggcaaggacgttgccctcatcactgacggccgcttctccggtggttctcacggcttcctcatcggccacattgtgcctgaggctatggagggtggccccattggtctcgtccgcgatggcgacaccatcgtcatcgacgccgagaagaaggttcttgaccttgaggttcctgaggaggagctcgccaagcgccgcaaggagtggaaggctcctgagcccaaggctaagcgtggtactctcaggaagtacgcccagctcgtcaaggatgctagctctggctgcgtcactgacgcttaa

SEQ ID NO: 55 - DNA, 1791 bp, Neurospora crassa
atgctcgctccctcccttctgcgggctcaggcccccagagctctggcttctgcccgtctctccctccgctccctctccaccactccccgccgctacaatgcccaggagaagcagctcaacaaggtgtcggccaacatcacccagcccaagtctcagggtgcttcccaggccatgctctacgccaccggcctcaacgaggacgacatgaacaaggcccaggtcggtatctcgtctgtctggtatgagggcaacccttgcaacatgcaccttctcgacctctccggcctcgtcaaggagtccgttgccaaggctggcctcgtccccatgcgcttcaacaccatcggtgtctcggacggtatctccatgggtaccaccggtatgcgctacagcttgcagtcccgtgagattattgccgactccatcgagaccgtcatgaacggccagtggtacgacgccaacatctccctccccggttgcgacaagaacatgcccggtgtcctcatcgccatgggccgtgtcaaccgcccctccatcatggtctacggtggtaccatcaagcccggctgcaacatgaagggcgagaacatcgatatcgtttccgccttccaggcatacggccagtacatctccggtgagatcgacgagaagcagcgcttcgatatcatccgcaacgcctgccctggtggtggcgcctgcggtggcatgtacactgccaacaccatggccaccgccatcgagaccctcggcatgacccttcccggttcctcctcctaccccgccgagtctcccgagaagaagaacgagtgcttgagcgtcggtgaggccatcaagaacctcctccgcgaggacatccgccccaccgatatccttactcgccaggccttcgagaacgccatgatcgtcgtcaacattcttggcggttccaccaacgccgtcctccacttgattgccatcgccgactccgttggcatcaagctcaccatcgatgacttccaggccgtctccgaccgcactcctttccttgccgacctcaagccttccggcaagtgggtcatggaggaccttagcaagattggcggcacccccgcccttctcaaattcctcctcaaggagggaatcctcgacggctccggcatcacctcgaccggtaagaccatgaaggagaacgtcgagaagttccccgacttccccaccgaccaggccatcatccgccctctgtcgaaccccatcaaggaaaccggacacattcagatcctccgcggctctctcgctcccggcggttccgtcggcaagatcaccggcaaggagggtctccgtttcgagggtaaggctcgctgcttcgactacgaggacggctttattgaggctctcgagcgtggcgagatcaagaagggcgagaagaccgtcgtcgtcatccgctacgagggtcccaagggcggccctggcatgcccgaaatgctcaagccttcgtccgccattatgggctacggtctcggcaaggacgttgccctcatcactgacggccgcttctccggtggttctcacggcttcctcatcggccacattgtgcctgaggctatggagggtggccccattggtctcgtccgcgatggcgacaccatcgtcatcgacgccgagaagaaggttcttgaccttgaggttcctgaggaggagctcgccaagcgccgcaaggagtggaaggctcctgagcccaaggctaagcgtggtactctcaggaagtacgcccagctcgtcaaggatgctagctctggctgcgtcactgacgcttaa

SEQ ID NO: 56 - protein, 596 aa, Neurospora crassa
MLAPSLLRAQAPRALASARLSLRSLSTTPRRYNAQEKQLNKVSANITQPKSQGASQAMLYATGLNEDDMNKAQVGISSVWYEGNPCNMHLLDLSGLVKESVAKAGLVPMRFNTIGVSDGISMGTTGMRYSLQSREIIADSIETVMNGQWYDANISLPGCDKNMPGVLIAMGRVNRPSIMVYGGTIKPGCNMKGENIDIVSAFQAYGQYISGEIDEKQRFDIIRNACPGGGACGGMYTANTMATAIETLGMTLPGSSSYPAESPEKKNECLSVGEAIKNLLREDIRPTDILTRQAFENAMIVVNILGGSTNAVLHLIAIADSVGIKLTIDDFQAVSDRTPFLADLKPSGKWVMEDLSKIGGTPALLKFLLKEGILDGSGITSTGKTMKENVEKFPDFPTDQAIIRPLSNPIKETGHIQILRGSLAPGGSVGKITGKEGLRFEGKARCFDYEDGFIEALERGEIKKGEKTVVVIRYEGPKGGPGMPEMLKPSSAIMGYGLGKDVALITDGRFSGGSHGFLIGHIVPEAMEGGPIGLVRDGDTIVIDAEKKVLDLEVPEEELAKRRKEWKAPEPKAKRGTLRKYAQLVKDASSGCVTDA

SEQ ID NO: 57 - DNA, 2549 bp, Neurospora crassa
atggcttccaaccaagataataaggctgtcgctcccgacgccgccgctcccgccggccagtccaccaccaccacgaccaccaacgacaacagcgagcgcaacttgcccaaagagggcgaatacatccaatggcgtactctcccagctggcaaccccgaccagttgaaccgctggtcgcactttttgactcgtgagcatgagtttcctggagctcaggtaagtaatgaagctattgccattgccatcatttgctcttgatgcttcctacctacctacctacctacctacctaccctcacgtcacgtcatgacatgacatgacaaattccgctgctctcacttattcattcactgccgtgcttaagcggatcccacacacactacacacatacccaacacctcgggttcaaaattggggtatcgttcggcaaaagaatcccgagcccgtccgtcactcgcttcgtcattcatatgccttatctggatgcggagatggttcatgaggggtagcttctgggctgctgtggggtaggtagtacagcttttgcgtggtacctacctctagacaacaaagtggaaaggggcatggaccccccctgacgacgatcatgaattacatacaacgggttgtaccaaagcaacatgaaaagctcggaataactaacatggcgattacccaaccatgtaggccatgctctacggcgccggtgtcccgaataaggacatgatgaagaaggcgccgcatgtcggcattgcgacggtctggtgggagggcaacccctgcaagtgagttgtacctacctacccaaaggaatgaatgagatgataggtggtgagagagggttgaggactaacgtggtggttatagcacccatttgctggatcttggccaaaaggtcaagaaggccgttgagcgcgagaagatgcttgcgtggcagttcaacaccattggtgtctcggatggtatcaccatgggcggagagggtgagtttttgtcacctccatgtctcaatgcgtaatatgaggttgaatgctaaaaccacatcacatcaaccaggcatgcgctactcgcttcagtctcgcgagatcatcgccgactccatcgagaccgtgacgtgcgctcagcatcatgacgccaacatctccattcccggctgcgacaagaacatgcccggtgtcatcatggccgcggcccgccacaaccgtcccttcgtcatgatctacggcggcaccatgcgcggcggccactcggagctcctcgaccgccccatcaacatcgtcacttgctacgaggcttccggcgcttacacgtatggccgtctcaagcccgcttgccccaacagcaccgccaccccttcggacgtcatggacgacatcgagcagcacgcctgccccggcgccggcgcctgcggcggcatgtacaccgccaacaccatggcgaccgccatcgaagccatgggcctcaccgcaccgggctcctcttctttccctgcctcttcgcccgaaaagttccgcgagtgcgaaaaagccgccgagtacatcaagatctgtatggagaaggacattcgcccgcgcgacctcttgaccaaggcctcgttcgagaacgccctcgtgctcaccatgatcctcggcggttctaccaatggcgttctgcactacctcgccatggccaactcggccgacgtcgacttgactctggacgacatcaaccgcgtctcagccaagacgccctttttggctgacatggccccctccggcaggtactacatggaagacctgtacaaagtcggcggcacccccgcggtcctcaagatgctcatcgccgcaggctatatcgatggcaccatccccactatcaccggcaagtctttggcggaaaacgtatccgactggcctagtctcgaccccgatcaaaaaatcatccgcccccttgacaaccccatcaagtcccaggggcatatccgtgtcttgtacggaaacttctcccccggtggtgcggtcgccaagattacaggcaaggaaggtctctccttcaccggcaaagcccgttgtttcaacaaggaattcgagctcgacgccgccttgaagaactcggaaattaccctcgagcaaggaaaccaggtcctcatcgtgcggtacgaaggacccaaaggcggtccaggtatgccagagcaactgaaggcttcagcggcgatcatgggcgcgggcctgacgaatgtcgcgctggtgacggatggtcggtactctggtgcgtcgcatgggtttattgtgggccatgtggtccccgaggcggcgacgggggggccgattgcgctggtgaaggacggggatttgattacgattgatgcggttaggaaccggattgatgtcgtcaagactgtggaaggcgtcgagggtgaggaagagattgccaaggtgttggaggagagaaagaaggggtggaaggcgccgaagatgaagccgacgaggggggcgttggccaagtatgcgaggttggtgggggatgcgtcgcatggggctgttactgatttgggtggggatgcctattag

SEQ ID NO: 58 - DNA, 1923 bp, Neurospora crassa
atggcttccaaccaagataataaggctgtcgctcccgacgccgccgctcccgccggccagtccaccaccaccacgaccaccaacgacaacagcgagcgcaacttgcccaaagagggcgaatacatccaatggcgtactctcccagctggcaaccccgaccagttgaaccgctggtcgcactttttgactcgtgagcatgagtttcctggagctcaggccatgctctacggcgccggtgtcccgaataaggacatgatgaagaaggcgccgcatgtcggcattgcgacggtctggtgggagggcaacccctgcaacacccatttgctggatcttggccaaaaggtcaagaaggccgttgagcgcgagaagatgcttgcgtggcagttcaacaccattggtgtctcggatggtatcaccatgggcggagagggcatgcgctactcgcttcagtctcgcgagatcatcgccgactccatcgagaccgtgacgtgcgctcagcatcatgacgccaacatctccattcccggctgcgacaagaacatgcccggtgtcatcatggccgcggcccgccacaaccgtcccttcgtcatgatctacggcggcaccatgcgcggcggccactcggagctcctcgaccgccccatcaacatcgtcacttgctacgaggcttccggcgcttacacgtatggccgtctcaagcccgcttgccccaacagcaccgccaccccttcggacgtcatggacgacatcgagcagcacgcctgccccggcgccggcgcctgcggcggcatgtacaccgccaacaccatggcgaccgccatcgaagccatgggcctcaccgcaccgggctcctcttctttccctgcctcttcgcccgaaaagttccgcgagtgcgaaaaagccgccgagtacatcaagatctgtatggagaaggacattcgcccgcgcgacctcttgaccaaggcctcgttcgagaacgccctcgtgctcaccatgatcctcggcggttctaccaatggcgttctgcactacctcgccatggccaactcggccgacgtcgacttgactctggacgacatcaaccgcgtctcagccaagacgccctttttggctgacatggccccctccggcaggtactacatggaagacctgtacaaagtcggcggcacccccgcggtcctcaagatgctcatcgccgcaggctatatcgatggcaccatccccactatcaccggcaagtctttggcggaaaacgtatccgactggcctagtctcgaccccgatcaaaaaatcatccgcccccttgacaaccccatcaagtcccaggggcatatccgtgtcttgtacggaaacttctcccccggtggtgcggtcgccaagattacaggcaaggaaggtctctccttcaccggcaaagcccgttgtttcaacaaggaattcgagctcgacgccgccttgaagaactcggaaattaccctcgagcaaggaaaccaggtcctcatcgtgcggtacgaaggacccaaaggcggtccaggtatgccagagcaactgaaggcttcagcggcgatcatgggcgcgggcctgacgaatgtcgcgctggtgacggatggtcggtactctggtgcgtcgcatgggtttattgtgggccatgtggtccccgaggcggcgacgggggggccgattgcgctggtgaaggacggggatttgattacgattgatgcggttaggaaccggattgatgtcgtcaagactgtggaaggcgtcgagggtgaggaagagattgccaaggtgttggaggagagaaagaaggggtggaaggcgccgaagatgaagccgacgaggggggcgttggccaagtatgcgaggttggtgggggatgcgtcgcatggggctgttactgatttgggtggggatgcctattag

SEQ ID NO: 59 - protein, 640 aa, Neurospora crassa
MASNQDNKAVAPDAAAPAGQSTTTTTTNDNSERNLPKEGEYIQWRTLPAGNPDQLNRWSHFLTREHEFPGAQAMLYGAGVPNKDMMKKAPHVGIATVWWEGNPCNTHLLDLGQKVKKAVEREKMLAWQFNTIGVSDGITMGGEGMRYSLQSREIIADSIETVTCAQHHDANISIPGCDKNMPGVIMAAARHNRPFVMIYGGTMRGGHSELLDRPINIVTCYEASGAYTYGRLKPACPNSTATPSDVMDDIEQHACPGAGACGGMYTANTMATAIEAMGLTAPGSSSFPASSPEKFRECEKAAEYIKICMEKDIRPRDLLTKASFENALVLTMILGGSTNGVLHYLAMANSADVDLTLDDINRVSAKTPFLADMAPSGRYYMEDLYKVGGTPAVLKMLIAAGYIDGTIPTITGKSLAENVSDWPSLDPDQKIIRPLDNPIKSQGHIRVLYGNFSPGGAVAKITGKEGLSFTGKARCFNKEFELDAALKNSEITLEQGNQVLIVRYEGPKGGPGMPEQLKASAAIMGAGLTNVALVTDGRYSGASHGFIVGHVVPEAATGGPIALVKDGDLITIDAVRNRIDVVKTVEGVEGEEEIAKVLEERKKGWKAPKMKPTRGALAKYARLVGDASHGAVTDLGGDAY

SEQ ID NO: 60 - DNA, 1770 bp, Candida albicans
atgagttttgtcaaatcttgcagagggtgtcttaggacatttgctacatccacaatcaagcatgagaaaaagttaaacaagtactcgtctattgtcacaggcgacccatctcagggtgcttcccaagcaatgctttatgccactggtttcagtgatgaagatttcgatcgtgcacaaatcggtgtcggttctgtttggtggtcaggtaacccatgtaacatgcacttgatggagttgaacaacaggtgttccgaatcagtcaacaaggccggcttaaaagccatgcaattcaactccattggtgtgtcggacggtatcaccaacggtactgaaggtatgaagtactctttacagctgagagaaattatcgccgactcctttgaaaccatgaccatggcccaactatatgacggtaacattgccattccttcatgtgataaaaatatgcccggggtattgatggctatgggaagacacaacagacctgccattatggtctatggtggtactatcttgcctggatctccaacttgcggaacccaaaaccctgctgtagccgacaaaatcgatatcattagtgctttccaatcctacggacaatacttgtcaaaacaaatcaacaacgaagaaagaatagatattgtcaaacatgcctgtccagggcccggtgcatgtggtggtatgtacactgccaacactatggcctctgcctctgaagtcttggggttgaccttaccattttcgtcctcgtccccggcagtctccaaagaaaaagccgaagagtgtgccaacgtcgggttcgccttaaagaatttgttagagttggacttgaaaccaagagatattgtcaccaaaaaatcatttgaaaacgctattgcttatatcattgccactggtggttctactaatgccgttttacatcttattgccattgcctcttcatttgacattactattactgttgacgatttccaaagaatctccgacaacactcccttgttggccgatttcaaaccatcgggtaaatacgttatggccgacttgcaaaatgtcggcggtacacctgctgttatgaaatacttgatcaaagaaggcattatcgacggtacccaattgagtgtcaccggtaaaaccatcaacgaaaacttggctaaacttgctgatttgcctgagggccaagacattgttagaccagtatcaaacccattgaagccaagtggccacttacaaatcttgaaaggtactttggctccagggtctgctgtcgctaaaatcaccggtaaagaaggtacttatttcaaaggtaaagctagagtattcaacgacgaaggtgcatttattgttgccttggaaaatggcgagatcaaaaaaggcgaaaaaacagtttgtgtgatcagatacgaaggtccaaaaggtggtccaggtatgccagaaatgttgaaaccttcgtctgcattaatgggttacgggttaggtaaagacgttgctttgttgactgatggtagattttcgggtggttcccacgggttccttattggccacattgttcctgaagccgctgaaggtggtccaattgctttggttgaagatggcgatattattgtcatcgacgcagacaataacaaaatcgatttgttggttgagccagacgtcttgaccgaaagaagaaaacactggacgcctccagaaccaagatacaaaagaggtactttggccaaatacgccaagttggtcagcgatgcatctaagggatgtgttacagatttataa

SEQ ID NO: 61 - protein, 589 aa, Candida albicans
MSFVKSCRGCLRTFATSTIKHEKKLNKYSSIVTGDPSQGASQAMLYATGFSDEDFDRAQIGVGSVWWSGNPCNMHLMELNNRCSESVNKAGLKAMQFNSIGVSDGITNGTEGMKYSLQSREIIADSFETMTMAQLYDGNIAIPSCDKNMPGVLMAMGRHNRPAIMVYGGTILPGSPTCGTQNPAVADKIDIISAFQSYGQYLSKQINNEERIDIVKHACPGPGACGGMYTANTMASASEVLGLTLPFSSSSPAVSKEKAEECANVGFALKNLLELDLKPRDIVTKKSFENAIAYIIATGGSTNAVLHLIAIASSFDITITVDDFQRISDNTPLLADFKPSGKYVMADLQNVGGTPAVMKYLIKEGIIDGTQLSVTGKTINENLAKLADLPEGQDIVRPVSNPLKPSGHLQILKGTLAPGSAVAKITGKEGTYFKGKARVFNDEGAFIVALENGEIKKGEKTVCVIRYEGPKGGPGMPEMLKPSSALMGYGLGKDVALLTDGRFSGGSHGFLIGHIVPEAAEGGPIALVEDGDIIVIDADNNKIDLLVEPDVLTERRKHWTPPEPRYKRGTLAKYAKLVSDASKGCVTDL

SEQ ID NO: 62 - DNA, 1758 bp, Saccharomyces cerevisiae
atgggcttgttaacgaaagttgctacatctagacaattctctacaacgagatgcgttgcaaagaagctcaacaagtactcgtatatcatcactgaacctaagggccaaggtgcgtcccaggccatgctttatgccaccggtttcaagaaggaagatttcaagaagcctcaagtcggggttggttcctgttggtggtccggtaacccatgtaacatgcatctattggacttgaataacagatgttctcaatccattgaaaaagcgggtttgaaagctatgcagttcaacaccatcggtgtttcagacggtatctctatgggtactaaaggtatgagatactcgttacaaagtagagaaatcattgcagactcctttgaaaccatcatgatggcacaacactacgatgctaacatcgccatcccatcatgtgacaaaaacatgcccggtgtcatgatggccatgggtagacataacagaccttccatcatggtatatggtggtactatcttgcccggtcatccaacatgtggttcttcgaagatctctaaaaacatcgatatcgtctctgcgttccaatcctacggtgaatatatttccaagcaattcactgaagaagaaagagaagatgttgtggaacatgcatgcccaggtcctggttcttgtggtggtatgtatactgccaacacaatggcttctgccgctgaagtgctaggtttgaccattccaaactcctcttccttcccagccgtttccaaggagaagttagctgagtgtgacaacattggtgaatacatcaagaagacaatggaattgggtattttacctcgtgatatcctcacaaaagaggcttttgaaaacgccattacttatgtcgttgcaaccggtgggtccactaatgctgttttgcatttggtggctgttgctcactctgcgggtgtcaagttgtcaccagatgatttccaaagaatcagtgatactacaccattgatcggtgacttcaaaccttctggtaaatacgtcatggccgatttgattaacgttggtggtacccaatctgtgattaagtatctatatgaaaacaacatgttgcacggtaacacaatgactgttaccggtgacactttggcagaacgtgcaaagaaagcaccaagcctacctgaaggacaagagattattaagccactctcccacccaatcaaggccaacggtcacttgcaaattctgtacggttcattggcaccaggtggagctgtgggtaaaattaccggtaaggaaggtacttacttcaagggtagagcacgtgtgttcgaagaggaaggtgcctttattgaagccttggaaagaggtgaaatcaagaagggtgaaaaaaccgttgttgttatcagatatgaaggtccaagaggtgcaccaggtatgcctgaaatgctaaagccttcctctgctctgatgggttacggtttgggtaaagatgttgcattgttgactgatggtagattctctggtggttctcacgggttcttaatcggccacattgttcccgaagccgctgaaggtggtcctatcgggttggtcagagacggcgatgagattatcattgatgctgataataacaagattgacctattagtctctgataaggaaatggctcaacgtaaacaaagttgggttgcacctccacctcgttacacaagaggtactctatccaagtatgctaagttggtttccaacgcttccaacggttgtgttttagatgcttga

SEQ ID NO: 63 - protein, 585 aa, Saccharomyces cerevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 64 - DNA, 33 bp, Artificial Sequence
  Synthetic primer
gacgacgacaagatgcttctctctcagaccaga

SEQ ID NO: 65 - DNA, 34 bp, Artificial Sequence
  Synthetic primer
gacgacgacaagatgaaagatagcgaaacggctt

SEQ ID NO: 66 - DNA, 38 bp, Artificial Sequence
  Synthetic primer
gaggagaagcccggttcattctacagagtcagtgatgc

SEQ ID NO: 67 - DNA, 25 bp, Artificial Sequence
  Synthetic primer
tgggttgtgtgacattgcgggagac

SEQ ID NO: 68 - DNA, 41 bp, Artificial Sequence
  Synthetic primer
gacgacgacaagatggactcctctacctccgcgtcgtccaa

SEQ ID NO: 69 - DNA, 38 bp, Artificial Sequence
  Synthetic primer
gacgacgacaagatgcatgaagatggcaccactgcgct

SEQ ID NO: 70 - DNA, 42 bp, Artificial Sequence
  Synthetic primer
gaggagaagcccggtctagaacaaatccgtcattgcaccatg

SEQ ID NO: 71 - DNA, 31 bp, Artificial Sequence
  Synthetic primer
acttgcgcgcagtatcatgatgcatgcattg

SEQ ID NO: 72 - DNA, 22 bp, Artificial Sequence
  Synthetic primer
caccgattggtggtatgacttc

SEQ ID NO: 73 - DNA, 22 bp, Artificial Sequence
  Synthetic primer
cctaattatcgcttcgctcagc

SEQ ID NO: 74 - DNA, 22 bp, Artificial Sequence
  Synthetic primer
atgcttctctctcagaccagag

SEQ ID NO: 75 - DNA, 22 bp, Artificial Sequence
  Synthetic primer
ggcatccttgacagttctacag

SEQ ID NO: 76 - DNA, 22 bp, Artificial Sequence; Synthetic primer
  Synthetic primer
gttagcagccgagatcttcaag

SEQ ID NO: 77 - DNA, 22 bp, Artificial Sequence
  Synthetic primer
atataccttgggtcagaagcgc

SEQ ID NO: 78 - DNA, 22 bp, Artificial Sequence
  Synthetic primer
cacggcgaagccaagtatatca

SEQ ID NO: 79 - DNA, 22 bp, Artificial Sequence
  Synthetic primer
atacgtcattgcaccatgagag

SEQ ID NO: 80 - DNA, 23 bp, Artificial Sequence
  Synthetic primer
gagccaatatgcgagaacacccg

SEQ ID NO: 81 - DNA, 20 bp, Artificial Sequence
  Synthetic primer
gtcagtatcatccccgcaat

SEQ ID NO: 82 - DNA, 20 bp, Artificial Sequence
  Synthetic primer
ggcacttcttcctcaactgc

SEQ ID NO: 83 - DNA, 20 bp, Artificial Sequence
  Synthetic primer; ILV
tggctgaggggagaactcta